(19) United States
(12) Patent Application Publication
(10) Pub. No.: US 2026/0144469 A1
(43) Pub. Date: May 28, 2026

(54) Measurement Of Energy For Daily Activities

(71) Applicant: Anhui Huami Information Technology Co., Ltd., Hefei (CN)

(72) Inventors: Mohammad M. Herzallah, Milpitas, CA (US); Mengfan Tang, Milpitas, CA (US); Kongqiao Wang, Hefei (CN)

(21) Appl. No.: 19/453,947

(22) Filed: Jan. 20, 2026

Related U.S. Application Data
(63) Continuation-in-part of application No. 18/820,790, filed on Aug. 30, 2024.
(60) Provisional application No. 63/821,016, filed on Jun. 10, 2025.

(51) Int. Cl. A61B 5/16; A61B 5/00; A61B 5/0205

(52) U.S. Cl. CPC A61B 5/165; A61B 5/0205; A61B 5/4815; A61B 5/681

(57) ABSTRACT

A method for dynamically determining an energy charge of a user. The method includes obtaining, by a processor, one or more sensor or physiological signals associated with the user and determining, by the processor, an estimation of a physical charge and a mental charge of the user based on the one or more sensor or physiological signals. The physical charge of the user is associated with physical energy consumption of the user, physical energy recovery of the user, or both, and the mental charge of the user is associated with mental energy consumption of the user, mental energy recovery of the user, or both. The method further includes determining, by the processor, the energy charge of the user based on the estimation of the physical charge and the mental charge of the user.

### CROSS-REFERENCE TO RELATED APPLICATIONS

This application is a continuation-in-part of U.S. application Ser. No. 18,820,790, filed on Aug. 30, 2024, and also claims priority to and the benefit U.S. Provisional Application Ser. No. 63/821,016, filed on Jun. 10, 2025, the contents of both of which are incorporated herein by reference in their entirety.

### TECHNICAL FIELD

This application relates to wearable computing, and in particular, to dynamic monitoring and measurement of a user's energy for daily activities.

### BACKGROUND

Modern technologies have provided users with wearable computing devices configured to sense and track a user's physical and mental parameters. Based upon such physical and mental parameters of the user, the wearable computing devices of associated computer-based system may compute health-related analyses to track the health of the user.

Current wearable computing devices may also compute analyses to link the detected physical and mental parameters of the user with health metrics of the user.

### SUMMARY

Disclosed herein are implementations of methods, apparatuses, and systems for measurement of energy and subsequent guidance.

In one aspect of the disclosure, a method for dynamically determining an energy charge of a user is disclosed. The method includes obtaining, by a processor, one or more sensor or physiological signals associated with the user and determining, by the processor, an estimation of a physical charge and a mental charge of the user based on the one or more sensor or physiological signals. The physical charge of the user is associated with physical energy consumption of the user, physical energy recovery of the user, or both, and the mental charge of the user is associated with mental energy consumption of the user, mental energy recovery of the user, or both. The method further includes determining, by the processor, the energy charge of the user based on the estimation of the physical charge and the mental charge of the user.

In some configurations, the energy charge of the user is determined in real-time as an energy charge value that is associated with an energy level of the user.

In some configurations, determining the energy charge of the user based on the estimation of the physical charge and the mental charge of the user includes modifying, in real-time, an initial energy charge of the user based on one or more activities detected based on the one or more sensor or physiological signals to obtain the energy charge.

In some configurations, the physical charge of the user is determined based on one or more heart-related metrics associated with the user and one or more movement measurements obtained by the at least one sensor. The one or more heart-related metrics are associated with a heart capacity of the user to return to a normal heart rate of the user.

In some configurations, determining the energy charge of the user based on the estimation of the physical charge and the mental charge of the user includes determining whether the user is in a state of physical energy depletion or a state of physical recovery based on at least one of an exertion metric or sleep data that is obtained based on the one or more sensor or physiological signals or determining whether the user is in a state of mental energy depletion or a state of mental energy repletion based at least in part on at least one of a stress level or sleep data that is obtained based on the one or more sensor or physiological signals.

In some configurations, the method includes obtaining a health estimation metric of the user based on the one or more sensor or physiological signals and modifying at least one of the physical charge or the mental charge based on the health estimation metric.

In some configurations, the health estimation metric of the user is obtained based on at least of heart health, breathing health, or body temperature of the user.

In some configurations, the heart health of the user is based on at least one of heart rate variability, an arrhythmia indication, or a rest heart rate, or the breath health of the user is based on at least one of an apnea-hypopnea index or a sleep apnea indication.

In some configurations, determining the estimation of the physical charge and the mental charge of the user based on the one or more sensor or physiological signals includes detecting a predefined event based on the one or more sensor or physiological signals and in response to occurrence of the predefined event being detected, determining the estimation of the physical charge and the mental charge of the user based at least in part on the detected predefined event.

In some configurations, the predefined event includes at least one of a significant or acute stress event, a supplemental sleep event or a workout event.

In some configurations, determining the estimation of the physical charge and the mental charge of the user based on the one or more sensor or physiological signals includes determining an exertion metric of a first time period based on the one or more sensor or physiological signals, wherein the exertion metric is associated with both daily activities and workout activities of the user and determining the estimation of the physical charge and the mental charge based at least in part on the exertion metric.

In some configurations, determining the estimation of the physical charge and the mental charge of the user based on the one or more sensor or physiological signals includes determining an exertion metric of a first time period based on the one or more sensor or physiological signals, tracking the exertion metric over a second time period to obtain a chronic stress metric, and determining the estimation of the physical charge and the mental charge based at least in part on the chronic stress metric.

In some configurations, the method further includes prompting the user to input a subjective estimation of at least one of the energy charge, the physical charge, and the mental charge and adjusting, by the processor, the energy charge based on the subjective estimation of at least one of the energy charge, the physical charge, and the mental charge.

In some configurations, the method further includes prompting the user to input whether the user completed a recovery activity and responsive to the user inputting that the recovery activity is completed, adjusting, by the processor, the energy charge based on completion of the recovery activity.

In some configurations, the method further includes providing guidance to the user based on the energy charge. The guidance includes at least one of information associated with the energy charge, information associated with the estimation of physical charge and the mental charge, instructions on how to address an underlying situation associated with the energy charge, and recommended activities.

In another aspect of the disclosure, a method for dynamically determining an energy charge of a user is disclosed. The method includes obtaining, by a processor, one or more sensor or physiological signals associated with the user, detecting, by the processor, a predefined event associated with the user based on the one or more sensor or physiological signals associated with the user, updating, by the processor, the energy charge of the user based on the detected predefined event.

In another aspect of the disclosure, a method for dynamically determining an energy charge of a user is disclosed. The method includes obtaining, by a processor, one or more sensor or physiological signals associated with the user, determining, by the processor, an estimation of a chronic stress of the user based on the one or more sensor or physiological signals, and determining, by the processor, the energy charge of the user based on the chronic stress of the user.

In another aspect of the disclosure, a method for dynamically determining an energy charge of a user is disclosed. The method includes: obtaining, by a processor, one or more sensor or physiological signals associated with the user; determining, by the processor, an estimation of at least one sleep metric the user based on the one or more sensor or physiological signals; determining, by the processor, an estimation of energy consumption or energy recovery of the user based at least in part on the estimation of at least one sleep metric; and determining, by the processor, the energy charge of the user based on the estimation of energy consumption or energy recovery.

In another aspect of the present disclosure, a computing device for dynamically determining an energy charge of a user is disclosed. The computer device includes a non-transitory memory and a processor configured to execute instructions stored in the non-transitory memory. The processor is configured to execute instructions stored in the non-transitory memory to obtain one or more sensor or physiological signals associated with the user, determine an estimation a physical charge and a mental charge based on the one or more sensor or physiological signals, and determine the energy charge of the user based on the estimation the physical charge and the mental charge. The physical charge of the user is associated with physical energy consumption of the user, physical energy recovery of the user, or both, and the mental charge of the user is associated with mental energy consumption of the user, mental energy recovery of the user, or both.

In some configurations the computing device may further include at least one sensor wearable by the user. The at least one sensor is configured to obtain the one or more sensor or physiological signals associated with the user.

In another aspect of the disclosure, a non-transitory computer-readable storage medium configured to store computer programs for dynamically determining an energy charge of a user is disclosed. The computer programs include instructions executable by a processor to obtain one or more sensor or physiological signals associated with the user, determine an estimation a physical charge and a mental charge based on the one or more sensor or physiological signals, and determine the energy charge of the user based on the estimation the physical charge and the mental charge. The physical charge of the user is associated with physical energy consumption of the user, physical energy recovery of the user, or both, and the mental charge of the user is associated with mental energy consumption of the user, mental energy recovery of the user, or both.

### BRIEF DESCRIPTION OF DRAWINGS

FIG. 1 is a perspective view of an example of a wearable device in accordance with the present teachings.

FIG. 2A is front perspective view of another example of a wearable device in accordance with the present teachings.

FIG. 2B is a rear perspective view of the wearable device shown in FIG. 2A.

FIG. 3 is a block diagram of an example of a computing device that may be used with or incorporated into a wearable device in accordance with the present teachings.

FIG. 4 is a block diagram illustrating components utilized to determine an energy charge of a user in accordance with the present teachings.

FIG. 5 is a flow diagram of an example of a method for dynamically determining an energy charge of a user in accordance with the present teachings.

FIG. 6 is a continuation of the flow diagram shown in FIG. 5.

FIG. 7 is an example of a plan for dynamically determining an energy charge of a user for a desired time interval in accordance with the present teachings.

FIG. 8 is a graph illustrating an example of an energy assessment provided to a user in accordance with the present teachings.

FIG. 9 is a block diagram illustrating subjective and objective components used to determine an energy charge of a user in accordance with the present teachings.

FIG. 10 is a flow diagram of an example of a method for dynamically determining a mental charge of a user in accordance with the present teachings.

FIG. 11 is a flow diagram of an example of a method for dynamically determining a physical charge of a user in accordance with the present teachings.

FIG. 12 is a flow diagram of an example of a method for dynamically determining a readiness component score in accordance with the present teachings.

FIG. 13 is a flow diagram of an example of a method for dynamically determining an energy charge of a user wearing a wearable device.

### DETAILED DESCRIPTION

Many portable devices and systems have been developed to monitor physiological conditions of an individual. One area of interest in the use of physiological monitors is personal wellness, physical, and mental metrics as integral parts of the overall health of an individual and may be evaluated to determine an overall state of the individual at a given point in time. For example, personal wellness, physical, and mental metrics may be used to determine if an individual is physically and/or mentally ready for a particular task or activity at a given point in time. That is, the personal, physical, and mental wellness of an individual may be used (e.g., evaluated) to assess an energy level of an individual to partake and/or complete a particular task or activity. By way of example, the personal wellness, physical, and mental metrics of an individual may be evaluated to determine the energy level of the individual to complete an exercise, a task at work, other activities, or a combination thereof. As such, the energy level may also correlate to or take into account a user's readiness (e.g., a user's readiness score) to partake and/or complete such a task or activity.

The energy level of an individual to partake and/or complete a particular activity at a given point in time may be determined by obtaining one or more physical and/or mental parameters of an individual (also referred to as a user herein) and associating such physical and/or mental parameters with the energy level of the individual, which may be referred to hereinafter as the energy charge of the individual. By way of example, one or more physiological conditions of an individual (e.g., heart-related metrics such as heart rate and/or heart rate variability (HRV)), skin conductance, respiration rate, blood pressure, pupil dilation, body temperature, etc.) may be obtained by a device worn by the user and the physiological conditions may be evaluated to generically determine the energy charge of the individual. The energy charge of the individual may be associated with an overall energy level of the individual at a given point in time. However, such portable devices and systems thereof may have certain shortcomings that hinder their overall usability. Similarly, such a generic determination of the energy level of an individual may have certain shortcomings that hinder the determined energy level from being particularly useful and/or insightful for the individual.

The present teachings provide an evaluation system, which may be used by or in conjunction with one or more portable devices (e.g., wearable devices), that dynamically monitors and tracks an energy charge (i.e., energy level) of an individual to partake in any given activity and/or task at a particular point in time. The present teachings may dynamically determine energy consumption and/or recovery of an individual based upon daily activities. For example, the present teachings may dynamically determine an individual's overall energy charge change from a previous day (e.g., based on activities completed in the previous day) to provide the individual with an energy charge value (e.g., an energy charge value ranging from 0 to 100 and/or 0% to 100%), whereby the individual may utilize the energy charge value to modify their daily activities, account for necessary recovery, and avoid excessive fatigue (i.e., burn-out). Moreover, the present teachings determine an energy charge for an individual based upon various components (e.g., physical stress, physical recovery, mental stress, mental recovery, heart health, breathing health, body temperature, sleep quality, etc.) that may contribute to the energy charge of the individual, whereby the various components may be determined (e.g., assessed) based upon the one or more physiological conditions obtained by the wearable device. Furthermore, the evaluation system may also provide an individualized assessment to the individual that combines both the aforementioned physiological components that contribute to the energy charge of the individual and the individual's subjective input.

Based on the above, the present disclosure provides a data-driven approach that utilizes data from a wearable device (e.g., a smart watch worn by the individual) to determine and assess an energy charge of an individual that could positively and/or negatively affect an individual's overall ability to complete particular activities and/or tasks in a given day. Implementations of this disclosure aim to help the individual successfully maintain a healthy physical and mental state and avoid burn-out caused by over-exertion. For example, based on the physiological conditions measured by the wearable device and the subjective feedback provided by the individual, the energy charge of the individual may be determined and provided to the individual to optimize their daily activities to thereby prevent over-exertion both physically and mentally. That is, the present disclosure provides an evaluation system that may be an evaluation and guidance system for the physical and mental health of the individual.

By dynamically monitoring physiological conditions of the individual and optionally receiving feedback directly from the individual (e.g., subjective feedback from the individual, which may be used to cross-validate and/or adjust the evaluation of the physiological conditions detected by the wearable device), the overall accuracy of the determined energy charge may be improved and further tailored to the individual. The energy charge may also be dynamically adjusted on a frequent and/or regular basis as needed to account for any discrepancies between the energy charged determined by the evaluation system described herein and the energy charge subjectively sensed by the individual. Thus, the energy charge provided to the individual may be tailored specifically to the individual and may produce a holistic view of the physical and/or mental health (e.g., physical and/or mental energy levels) of the individual that accounts for both objective data (e.g., the energy charge determined based upon the physiological conditions detected by the wearable device) and subjective data (e.g., feedback from the individual).

Moreover, according to implementations of this disclosure, sensor data for the individual can be collected, for example, by the portable device (e.g., the wearable device). The collected sensor data for the individual may be extracted to obtain physiological conditions of the individual, such as heart rate and/or heart rate variability (HRV), sleep analyses (such as determine whether the individual is sleeping or awake, sleep duration, sleep interruptions, or the like), exercise analyses (such as exercise intensity, exercise duration, exercise type, or the like), body temperature, skin conductance, respiration rate, blood pressure, pupil dilation, stress level (e.g., a severity of stress), an electrocardiogram (ECG), an electroencephalogram (EEG), an electrooculogram (EOG), an electromyogram (EMG), an electrodermal activity (EDA), or a combination thereof. Based upon the data extracted and physiological conditions determined, the energy charge of the individual (e.g., the energy charge of the individual that reflects the energy level of the individual to partake in and/or complete a given activity and/or task, whereby the energy charge may be based on the physical and/or mental energy charges of the individual) may be accurately determined, which may be utilized by the individual to plan their daily activities and maintain a healthier lifestyle and overall physical and mental wellbeing.

To describe some implementations in greater detail, reference is first made to examples of the system for measurement of a user's energy for daily activities. The system includes a wearable device, a server device, and an intermediate device. The intermediate device is connected between the wearable device and the server device.

The wearable device is a computing device configured to be worn by a user. The wearable device may be implemented as a wrist-worn device worn on the user's wrist (e.g., a wristband, a smartwatch, etc.), or may be implemented as a band or ring worn on the user's arms (e.g., an armband), fingers, legs, feet or torso, or may be implemented as a head-mounted device worn on the user's head or eyes (e.g., a headband, a headset, glasses, etc.), an ear-worn device worn on the user's ears, or a wearable device incorporated in the user's clothing or accessories.

The wearable device may include a securing mechanism for securing the wearable device to the user while in use. For example, the securing mechanism may be or include a slot and peg configuration, a snap-lock configuration, a buckle mechanism, adhesive fastener, a band, chains, or the like.

Furthermore, the wearable device may include one or more components for monitoring, measuring, or otherwise generating data, such as based on characteristics of a user of the wearable device, the environment in which the user is located, or both. For example, the wearable device may include one or more sensors and/or input elements. The sensors may be configured to measure information about conditions of the wearable device, the user thereof, or both. The sensors may be configured to measure user's physical and/or mental activity or condition, or an environmental condition in the vicinity of the user. For example, the sensors may include at least one of a movement sensor (e.g., an accelerometer, a gyroscope, etc.), an environmental sensor, a physiological sensor, an ultrasonic sensor, an infrared sensor, or the like. The physiological sensor may include a photoplethysmography (PPG) sensor, a pulse wave sensor, a heart sensor, a blood pressure sensor, a sleep sensor, an electrocardiogram (ECG) sensor, a body temperature sensor, a stress sensor or the like, or a combination thereof. The physiological sensor may be configured to measure physiological parameters or status of the user. For example, the physiological parameters may include at least one of heart rate, heart rate variability (HRV), blood oxygen saturation, blood pressure, blood glucose, body temperature, respiration rate, physiological pressure, sleep condition, and/or the like. The input elements may be configured to receive input from the user of the wearable device. For example, the input elements may include one or more of a physical button, a touch screen, a gesture receiving mechanism or the like, or a combination thereof.

The sensors and/or the input elements may be configured to generate data based on an event that occurred with respect to the wearable device, the user of the wearable device, or both. Examples of events can include the user of the wearable device carrying out a specific activity (e.g., workout, sleeping, taking a nap, an acute or severe stress event, etc.), entering to a designated physical location(e.g., gym or home, etc.), experiencing a particular health issue or the like. The wearable device may generate data indicative of an event using measurements taken by the sensors and/or based on interactions with the input elements. For example, data can be generated based on a movement measurement from the sensors, a physiological measurement from the sensors, a physical button of the input elements being pressed, an interaction with a user interface or the like.

Aspects of the sensors and/or the input elements may be configured to be associated with specific types of events. For example, a single physical button press or an election of a first interface element on the touch screen may be associated with a first event (e.g., the user of the wearable device starting to work out or taking a particular activity), while another configurable pattern of presses thereof or an election of a second interface element on the touch screen may be associated with a second event (e.g., the user of the wearable device is experiencing a health problem). In another example, gestures recognized using one or more of the sensors may be associated with events. For example, a first gesture of a user of the wearable device raising his or her wrist may be measured and associated with a first event, whereas a second gesture of the user dropping his or her wrist may be measured and associated with a second event.

In yet another example, measurements taken by the sensors may be associated with events, such as by default. For example, a low reading by a barometer can be associated with the user of the wearable device arriving in a low-pressure environment. In another example, accelerated heart rate readings by a PPG or an ECG can indicate that the user is experiencing a medical event requiring attention.

The sensors may include one or more movement sensors. The movement sensor may be configured to detect the magnitude of acceleration of the wearable device in various directions (e.g., three axes, six axes, etc.). When the user wears the wearable device, the wearable device moves under the drive of the user, and thus the magnitude of acceleration in each direction detected by the movement sensor can reflect the user's motion state.

In some other implementations, the wearable device may include one or more processing units configured to process measurement signals or data collected by the one or more sensors. Different processing units may be independent devices or integrated in one or more processors. For example, a processor may include an application processor (AP), a modem processor, a graphics processing unit (GPU), an image signal processor (ISP), a controller, a digital signal processor (DSP), a baseband processor, an AI processing unit, a micro-controller unit, etc.

In some other implementations, the wearable device may further include a communication unit for communication with external systems or devices. The communication unit may include signal and/or data transmitting and receiving circuits, such as antennas, amplifiers, filters, mixers, oscillators, digital signal processors (DSPs), and the like. The communication unit may establish communication wirelessly by utilizing radio frequency (RF) signals and/or data that comply with communication standards such as cellular 2G, 3G, 4G, LTE, or 5G, Institute of Electrical and Electronics Engineers (IEEE) 902.11 standard such as Wi-Fi, IEEE 902.16 standard such as WiMAX, Bluetooth™, or a combination thereof.

In some other implementations, the wearable device may further include a display screen. The display screen may be configured to display a user interface allowing the user to directly interact with the wearable device. The display screen includes a display panel. The display panel may be a liquid crystal display (LCD), a light-emitting diode (LED), an organic light-emitting diode (OLED), an active-matrix organic light emitting diode (AMOLED), a flexible light-emitting diode (FLED), a MiniLED, a MicroLED, a Micro-OLED, a quantum dot light emitting diode (QLED), etc. In some implementations, the display screen may include a touch sensor and be realized as a touch screen. The touch screen may allow the user to interact with the wearable device by physically touching, swiping, or gesturing on areas of the display screen.

In some other implementations, the wearable device may further include an audio device, which may include devices such as a microphone, a speaker, or an earpiece that can receive or output sound signals.

The server device is a computing device that runs a server program to process measurement signals or data. The server device may be or may include a hardware server (e.g., a server device), a software server (e.g., a web server and/or a virtual server), or both. For example, in the case that the server device is or includes a hardware server, the server device may be a server device located in a rack.

The server program is configured to use the measurement signals or data to detect one or more of a health state, an exercise state, and a sleep state of the user of the wearable device, or any combination thereof. For example, the server program may receive the measurement signals or data from the intermediate device and then use the received measurement signals or data to detect one or more of the health state, the exercise state, and the sleep state of the user of the wearable device, or any combination thereof. For example, the server program may use the measurement signals or data to determine a user state or a change in the user state, and then detect at least one of the health state, the exercise state, or the sleep state of the user of the wearable device, or any combination thereof based on the determined user state or change in the user state.

The server program may access a database in the server device to perform at least some of the functions of the server program. The database is a database or other data storages configured to store, manage, or otherwise provide data used for fulfilling the functions of the server program. For example, the database may store physiological signals or data received by the server device and information generated from or otherwise determined from the physiological signals or data. For example, the database may be a relational database management system, an object database, an XML database, a configuration management database, a management information base, one or more flat files, other suitable non-transitory storage mechanisms, or any combination thereof.

In some implementations, the server device may be a virtual server. For example, the virtual server may be implemented with a virtual machine (e.g., a Java virtual machine). The virtual machine may be implemented with one or more virtual software systems, such as HTTP servers, java servlet containers, hypervisors, or other software systems. In some of such implementations, the one or more virtual software systems used to implement the virtual server may instead be implemented in hardware.

The intermediate device is a device configured to facilitate communication between the wearable device and the server device. In particular, the intermediate device receives data from the wearable device, and sends the received data to the server device, for use by the server program for example. The intermediate device may be a computing device, such as a mobile device (e.g., a smart phone, a tablet computer, a laptop computer, a personal digital assistant or other types of portable devices), or a fixed device (e.g., a desktop computer, a router device, a gateway device, etc.). As another alternative, the intermediate device may be other types of network connection devices. For example, the intermediate device may be a networked power charger for the wearable device.

For instance, depending on particular implementations of the intermediate device, the intermediate device may run an application program. The application program may be one or more software applications installed on the intermediate device. In some implementations, the application software may be installed on the intermediate device by a user of the intermediate device (which is typically the same as the user of the wearable device, and may not be the same as the user of the wearable device in certain cases) after the intermediate device is purchased. Alternatively, the application software may be installed on the intermediate device in advance by a manufacturer of the intermediate device before the intermediate device leaves the factory. The application program may be configured to send data to the wearable device or receive data from the wearable device, and/or, to send data to the server device or receive data from the server device. The user may use the application program to configure functionality of the wearable device. For example, the user may use the application program to set one or more user interfaces associated with the measurement and monitoring of the user. The application program may receive a command from the user of the intermediate device. The application program may receive the command from the user through a user interface of the application program. For example, in the case that the intermediate device is a computing device with a touch screen display, the user of the intermediate device may issue the command by touching at least a portion of the display that corresponds to a user interface element in the application program.

For example, the command received by the application program from the user of the intermediate device may be a command for transmitting the physiological signals or data received at the intermediate device (e.g., from the wearable device) to the server device. The intermediate device sends the physiological signals or data to the server device in response to the command. In some other examples, the command received by the application program from the user of the intermediate device may be a command for checking information received from the server device, such as, for instance, information about one or more of the detected health status, exercise status, and sleep status of the user of the wearable device, or any combination thereof.

In some implementations, the intermediate device is a client device given access to the server program. The application program may be a client application capable of communicating with the server program. For instance, the client application may be a mobile application capable of accessing some or all of the functions and/or data of the server program. For instance, the client device may communicate with the server device over a network.

In some implementations, the intermediate device receives data from the wearable device by using a short-range communication protocol. For example, the short-range communication protocol may be Bluetooth®, Bluetooth® Low Energy, Infrared, Z-Wave, ZigBee, or other types of protocols, or any combination thereof. The intermediate device sends the data received from the wearable device to the server device over the network. For example, the network may be a local area network, a wide area network, a machine-to-machine network, a virtual private network, or other types of public or private networks. The network may use a remote communication protocol. For example, the remote communication protocol may be Ethernet, TCP, IP, Powerline Communications, Wi-Fi, GPRS, GSM, CDMA, other types of protocols, or any combination thereof.

The system may be configured to continuously transmit the physiological signals or data from the wearable device to the server device. The one or more sensors may continuously or otherwise frequently and periodically collect the measurement signals or data from the user of the wearable device.

Implementations of the system may differ from those described above. In some implementations, the intermediate device may be omitted. In some examples, the wearable device may be configured to communicate directly with the server device over the network. For instance, a direct communication between the wearable device and the server device over the network may include the use of a remote low-power system or other types of communication mechanisms. In some other implementations, both the intermediate device and the server device may be omitted. For example, the wearable device may be configured to perform the above-mentioned functions of the server device. In Such implementations, the wearable device may process and store data on its own without needing other computing devices.

The method provided by the present disclosure may be performed by an electronic device. Specifically, the method may be performed by a wearable device, or may be performed by another computing device. In some examples, the wearable device may send the collected data to the intermediate device, and the intermediate device performs the method. In some other examples, the wearable device may directly send the collected data to the server device, and the server device performs the method. In some other examples, the wearable device may send the collected data to the intermediate device, the intermediate device sends the data to the server device, and the server device performs the method. Alternatively, the method according to the present disclosure may be completed jointly by a plurality of devices. In some examples, a part of operations or steps are performed by the wearable device, and the other part of operations or steps are performed by the intermediate device. In some other examples, a part of the operations or steps are performed by the wearable device, and the other part of the operations or steps are performed by the server device. In some other examples, a part of the operations or steps are performed by the intermediate device, and the other part of the operations or steps are performed by the server device, which is not limited to the present disclosure.

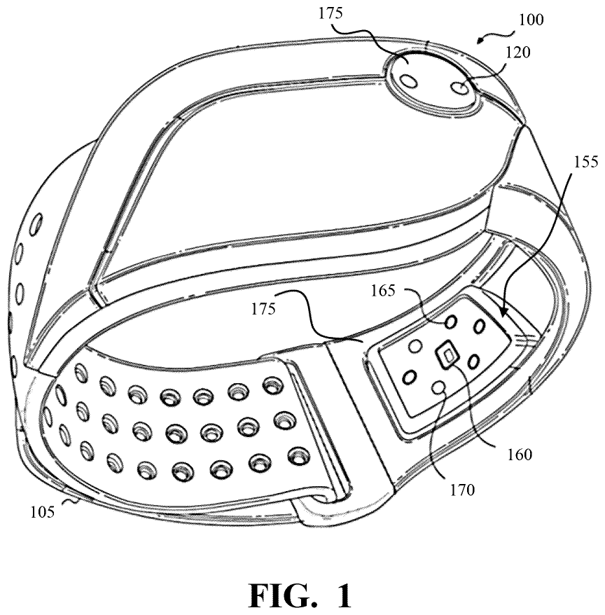

FIG. 1 depicts a perspective view of an example device 100 according to some implementations of this disclosure. The device 100 may be a wearable device worn by an individual (also referred to herein as a user) to at least one of sense, collect, monitor, analyze, or display information pertaining to one or more of a physiological parameter of the individual or an environmental parameter captured by the device 100 in a vicinity of the individual. The device 100 can include, for example, a head-mounted device, a wristband, a ring, a strap (e.g., a chest strap), headphones or a wristwatch. Although depicted in FIG. 1 as a wristwatch having a band 105, the device 100 can include the wearable device configured for positioning at a user's wrist, arm, finger, chest, another extremity of the user, or some other area of the user's body, such as a wearable camera. For example, the device 100 can be a wearable camera having an image sensor with capabilities such as high-speed shooting performance, low-light or dark shooting performance, high image quality, or anti-shake performance, among other things. In addition, the device 100 can be equipped with other sensors as discussed below (e.g., a photoplethysmography (PPG) sensor to detect heart rate, altitude sensor, a temperature sensor, a humidity sensor, etc.).

The device 100 may include sensors and processing tools for detecting, collecting, processing, displaying, or a combination thereof one or more physiological parameters of the individual and/or other information that may or may not be related to health, wellness (e.g., mental health and wellness and/or physical health and wellness), exercise, sleep, or physical training sessions (e.g., characteristic information, education information, etc.). The physiological parameters can include at least one of: heart rate, heart rate variability (HRV), blood pressure, blood glucose level, respiration rate, body temperature, electrocardiogram (ECG) measurements, electroencephalogram (EEG) measurements, electrooculogram (EOG) readings, electromyogram (EMG) readings, electrodermal activity (EDA) measures, sleep state, sleep phase, mental state, stress state, or other physiological information that can be measured for the individual.

In some examples, the device 100 may include a motion sensor, a pulse sensor, and a temperature sensor. The motion sensor may include at least one of an accelerometer, a gyroscope, a magnetometer sensor, or the like. The motion sensor may be configured to detect motion data of the user, and the motion data of the user may be used for determining, solely or together with other sensor data, an activity type such as exercising, resting, sleeping or the like, analyses of the activity the user is participated in or the like. The pulse sensor may include a photoplethysmography (PPG) sensor, an ECG sensor, or the like. The pulse sensor may be configured to detect pulse data of the user, and the pulse data may be used for determining, solely or together with other sensor data, a heart rate, HRV, a stress level, an activity type, a temperature prediction, a respiration rate, or the like. The temperature sensor may be configured to obtain skin temperature data of the user, and the skin temperature data may be used for predicting a core body temperature of the user. The device 100 may also include other sensors, and no limitation is set herein.

The device 100 may also include sensors and processing tools for detecting, collecting, processing, or displaying one or more environmental parameters captured by the device 100 in a vicinity of the individual.

The environmental parameters can include, for example, positioning information, location, altitude, temperature, humidity, environmental light, weather, environmental pollution index such as PM2.5 particulate matter content or CO2/CO content, which can be captured by, for example, one or more environmental sensors of the device 100. The environmental parameters can also include motion data such as motion tracks from a GPS sensor and/or a motion sensor (e.g., one or more of an accelerometer, gyroscope, magnetometer, etc.) or a barometer to record additional measurement data such as altitude. The environmental parameters can include at least one of: altitude, GPS location, ambient temperature, ambient humidity, ambient light, ambient noise index, an environmental pollution index, or other environmental parameters in the vicinity of the individual that can be captured by the at least one wearable device.

The device 100 may further include one or more communication modules. One or more communication modules may also communicate with other devices such as a personal device of the user (e.g., a handheld device, a smart phone, a tablet, a laptop computer, a desktop computer, or the like) or a server (e.g., a cloud-based server). The communications can be transmitted wirelessly (e.g., via Bluetooth, RF signal, Wi-Fi signal, near field communications, etc.) or through one or more electrical connections embedded in the band 105. Analog information collected or analyzed can be translated to digital information for reducing the size of information transfers between modules.

As shown in FIG. 1, the device 100 can include a sensor unit 155 including at least one of, but not limited to: an image sensor, such as a camera; one or more physiological sensors, such as a PPG, EEG, EOG, or EMG sensor including one or more optical detectors 160 and one or more light sources 165; one or more contact pressure/tonometry sensors 170; or one or more motion sensors including at least one of the one or more gyroscopes or accelerometers 175. These sensors are only illustrative of the possibilities, however, and additional or alternative sensors such as one or more acoustic sensors, electromagnetic sensors, ECG electrodes 120, bio impedance sensors, galvanic skin response, or a combination thereof may be included. The device 100 may also include one or more such sensors and components on an inside surface (i.e., the surface in contact with the user's tissue or targeted area). It should be understood that the device 100 can be implemented with a different configuration of the sensor unit 155 from what is depicted in FIG. 1 or the examples of the disclosure.

The location of the sensor unit 155 and/or the location of one or more sensor components of the sensor unit 155 with respect to the user's tissue may be customized to account for differences in body type across a group of users or placement in different locations on a user.

The displacement values and additional data collected from the sensor unit 155 may assist a non-transitory computer readable medium or processor in isolating various physiological conditions (e.g., heart beats, respiration, etc.). The processor may receive data from the sensor unit 155. The processor may dynamically filter the data. The processor may analyze the data without regard to a position of the device 100 relative to the user or a position of the user. The processor may filter unwanted signals and isolate only desired signals. For example, the processor may learn which signals are of interest (e.g., which signals are associated with physiological conditions of interest, which may form a basis to determine one or more emotions of a user), and the process may analyze only those signals of interest. The processor may be in communication with or include a non-transitory computer-readable medium.

The sensor unit 155 may be configured to continuously collect data from a user. However, certain techniques can be employed to reduce power consumption and conserve battery life of the device 100. For example, while the PPG sensor can be used to continuously monitor blood flow of the user, the ECG electrodes 120 can be used periodically or intermittently to collect potentially more accurate blood flow information which can be used to supplement or calibrate the PPG measurements collected and analyzed by the processor.

For example, when the data from one or more accelerometers or gyroscopic components of the device 100 indicates that a user is still or at rest, one or more sensors of the device 100, such as the PPG sensor, which consumes more power than the one or more accelerometers or gyroscopic components, may be turned off to conserve power consumption. However, when the data from the one or more accelerometers or gyroscopic components of the device 100 indicates that the user is exercising, the one or more sensors of the device 100, such as the PPG sensor, may be turned on to measure the heart rate and/or other physiological parameters of the user. In another example, when the data from one or more accelerometers or gyroscopic components of the device 100 indicates that a user is sleeping and the sleep analysis function is turned on, the one or more sensors, such as the PPG sensor, may still need to be turned on even though the movement of the user from the one or more accelerometers or gyroscopic components of the device 100 is minimal during sleep.

The device 100 may also include an input and/or an output unit, such as a display unit, sound unit, tactile unit, or the like, for communicating information to the user (i.e., the wearer of the device 100). The display unit may be configured to display the images or videos captured by the sensors such as the image sensor, notifications, or alerts. The display unit may be an LED indicator including a plurality of LEDs, each a different color. The LED indicator can be configured to illuminate in different colors depending on the information being conveyed. For example, where the device 100 is configured to monitor the user's heart rate, the display unit may illuminate light of a first color when the user's heart rate is in a first numerical range, illuminate light of a second color when the user's heart rate is in a second numerical range, and illuminate light of a third color when the user's heart rate is in a third numerical range. In this manner, a user may be able to detect his or her approximate heart rate at a glance, even when numerical heart rate information is not displayed at the display unit, and/or the user only sees the device 100 through the user's peripheral vision (e.g., while exercising).

The display unit may include a display screen for displaying images, characters, graphs, waveforms, or a combination thereof to the user or a medical professional. By way of example, the device 100 may be configured to detect one or more physiological conditions of the user and provide an energy charge (i.e., an energy charge value) of the user (e.g., determine the energy charge of the user to partake and/or complete a given activity based on the one or more physiological conditions detected), whereby the energy charge may be or may include information displayed on the display screen. The display unit may further include one or more hard or soft buttons or switches configured to accept input by the user. Similarly, the display screen may be a touch screen configured to accept input by the user. The display unit may also switch or be toggled between displaying information.

The physiological or environmental information discussed above may be graphically displayed or represented on a display of the device 100. The graphical display may be provided as an output. The output may include physiological or environmental information of a user. For example, the information collected may be categorized and then graphically represented as one or more outputs. The output may include an alert, guidance, or a suggestion for the user. The output may also include education information pertaining to topics of interest for the user.

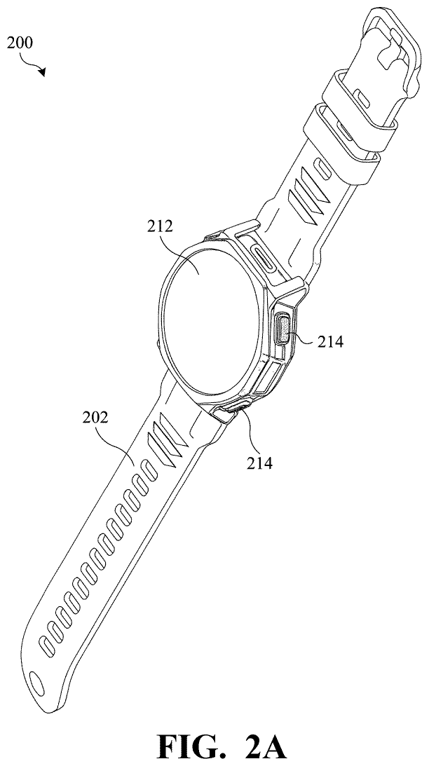

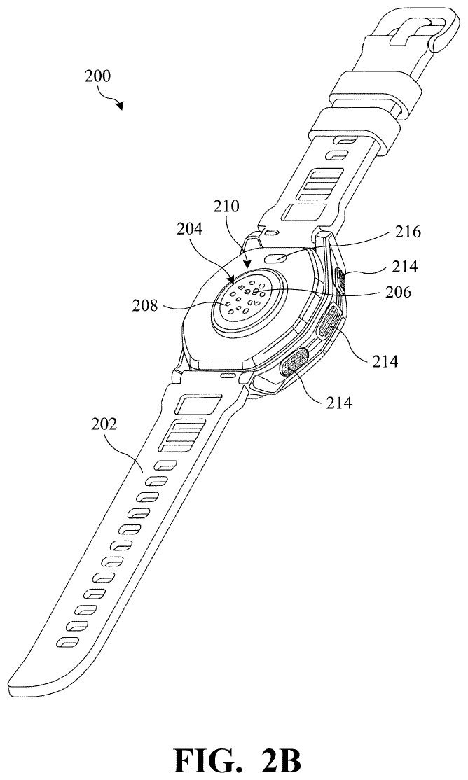

FIG. 2A depicts a front perspective view of another example of a device 200 according to some implementations of this disclosure. FIG. 2B depicts a rear perspective view of the device 200. The device 200 may be similar to the device 100 of FIG. 1. For example, the device 200 may be a wearable device worn by a user to at least one of sense, collect, monitor, analyze, or display information pertaining to one or more physiological parameters of the user or an environmental parameter captured by the device 200 in a vicinity of the user. The device 200 may include, for example, a wristband, a strap (e.g., a chest or arm strap), secondary devices (e.g., a head-mounted device, headphones, etc.), or a combination thereof. As shown in FIG. 2A, the device 200 may be configured as a wristwatch having a band 202. The device 200 may be configured for positioning at the user's wrist, arm, chest, another extremity of the user, or a combination thereof.

The device 200 may include sensors and processing tools for detecting, collecting, processing, displaying, or a combination thereof one or more physiological parameters of the individual and/or other information that may or may not be related to health, wellness (e.g., mental health and wellness and/or physical health and wellness), exercise, sleep, or physical training sessions (e.g., characteristic information, education information, etc.). The physiological parameters can include at least one of: heart rate, heart rate variability (HRV), blood pressure, blood glucose level, respiration rate, body temperature, electrocardiogram (ECG) measurements, electroencephalogram (EEG) measurements, electrooculogram (EOG) readings, electromyogram (EMG) readings, electrodermal activity (EDA) measures, sleep state, sleep phase, mental state, stress state, or other physiological information that can be measured for the individual.

Similar to the device 100 described above, the device 200 may include one or more of a motion sensor, a pulse sensor, or a temperature sensor. Such sensors may operate as described above with respect to the device 100. Moreover, the device 200 is not limited to the aforementioned sensors and may include any combination of sensors. Furthermore, the device 200 may also include sensors and processing tools for detecting, collecting, processing, or displaying one or more environmental parameters, such as those described above with respect to the device 100, captured by the device 200 in a vicinity of the individual.

The device 200 may further include one or more communication modules. One or more communication modules may also communicate with other devices such as a personal device of the user (e.g., a handheld device, a smart phone, a tablet, a laptop computer, a desktop computer, or the like) or a server (e.g., a cloud-based server). The communications can be transmitted wirelessly (e.g., via Bluetooth, RF signal, Wi-Fi signal, near field communications, etc.) or through one or more electrical connections embedded in the band 202. Analog information collected or analyzed can be translated to digital information for reducing the size of information transfers between modules.

As shown in FIG. 2B, the device 200 may include a sensor unit 204 including at least one of, but not limited to: an image sensor, such as a camera; one or more physiological sensors, such as a PPG, EEG, EOG, or EMG sensor including one or more optical detectors 206 and/or one or more light sources; one or more contact pressure/tonometry sensors 208; or one or more motion sensors including at least one of gyroscopes or accelerometers 210. The aforementioned sensors are only an illustrative example, and as such, additional or alternative sensors may be implemented in the device 200 (e.g., impedance sensors, galvanic skin response sensors, etc.) For example, such sensors may be disposed along an inside surface of the device 200 (e.g., a surface in contact with the user's tissue or targeted area). As such, the location of the sensor unit 204 and/or the location of one or more sensor components of the sensor unit 204 with respect to the user's tissue may be customized to account for differences in body types or placement in different locations on a user.

The displacement values and additional data collected from the sensor unit 204 may assist a non-transitory computer readable medium or processor in isolating various physiological conditions (e.g., heart beats, respiration, etc.). The processor may receive data from the sensor unit 204. The processor may dynamically filter the data. The processor may analyze the data without regard to a position of the device 200 relative to the user or a position of the user. The processor may filter unwanted signals and isolate only desired signals. For example, the processor may learn which signals are of interest (e.g., which signals are associated with physiological conditions of interest, which may form a basis to determine one or more emotions of a user), and the process may analyze only those signals of interest. The processor may be in communication with or include a non-transitory computer-readable medium.

The device 200 may also include an input and/or an output unit, such as a display unit 212, sound unit (e.g., speaker and/or microphone), tactile unit (e.g., one or more buttons, knobs, switch, or similar tactile units, such as the buttons 214 shown in FIGS. 2A and 2B), or the like, for communicating information to the user and/or for receiving information from the user (e.g., an input command). The display unit 212 may be a screen (e.g., a touchscreen), which may display various information to the user, such as values associated with one or more physiological conditions of the user. As such, the display unit 212 may provide the user with an easy manner of receiving information from the device 200 even during activity (e.g., exercise).

To further illustrate, the display unit 212 may include a display screen for displaying images, characters, graphs, waveforms, or a combination thereof to the user or a medical professional. By way of example, the device 200 may be configured to detect one or more physiological conditions of the user and provide an energy charge (i.e., an energy charge value) of the user (e.g., determine the energy charge of the user to partake and/or complete a given activity based on the one or more physiological conditions detected), whereby the energy charge may be or may include information displayed on the display screen. The display unit 212 may further include one or more hard or soft buttons or switches, such as the buttons 214, which may be configured to accept input by the user. Similarly, the display screen may be a touch screen configured to accept input by the user. The display unit 212 may also switch or be toggled between displaying information.

The device 200 may further include one or more charging contacts, such as the charging contact 216. The charging contact 216 may be positioned on the rear surface of the device 200 to facilitate electrical coupling with an external charging dock or cable. The charging contacts may comprise conductive pads and/or pins formed from a corrosion-resistant metal, such as gold-plated stainless steel or other metals, to ensure reliable long-term connectivity. When the device 200 is placed on a corresponding charging interface (e.g., the charging dock), spring-loaded terminals or magnetic alignment features may be included in the device 200 to maintain secure contact pressure therebetween, thereby allowing consistent power transfer from between a power source (e.g., a wall outlet) and the device 200. The charging contacts may also interface with internal circuitry of the device 200 that may regulate current flow and/or voltage to safely recharge a power source (e.g., a battery) within the device 200 while preventing overcurrent or short-circuit conditions. Such a configuration of the charging contacts may enable efficient, repeatable charging without requiring disassembly or direct access to the power source of the device 200.

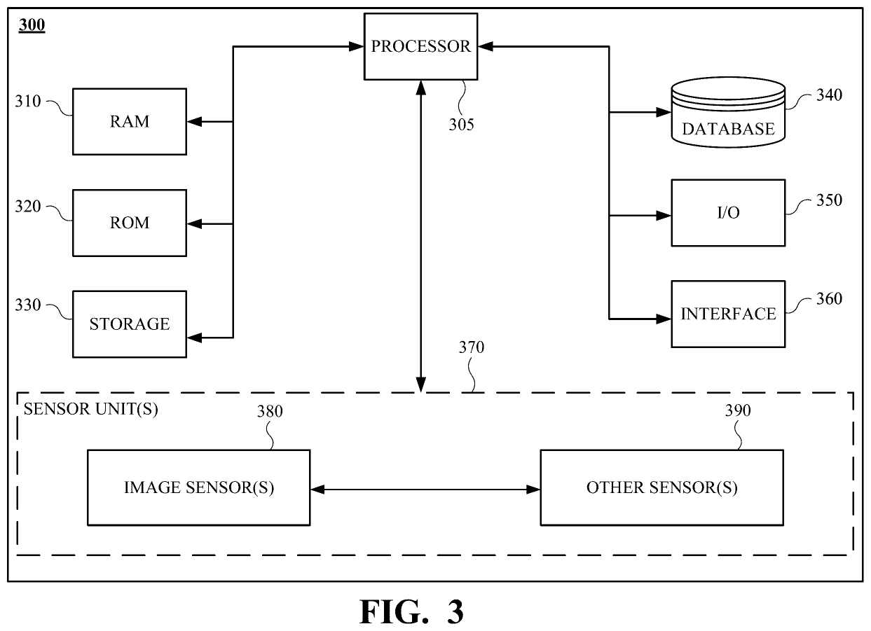

FIG. 3 depicts an example of a computing device 300 that may be used with or incorporated into a wearable device. The computing device 300 is representative of the type of computing device that may be present in or used in conjunction with at least some aspects of the device 100 or the device 200, or any other device comprising electronic circuitry. For example, the computing device 300 may be used in conjunction with any one or more of: transmitting signals to and from the one or more optical sensors or acoustical sensors; sensing or detecting signals received by one or more sensors of the device 100; processing received signals from one or more components or modules of the device 100, the device 200, or a secondary device; and storing, transmitting, or displaying information. The computing device 300 may be or may be included within the device 100. The computing device 300 may be a mobile terminal or remote device that is in communication with the device 100 or the device 200. The computing device 300, the device 100, the device 200, or a combination thereof may be in communication with a server (e.g., a cloud-based server). For example, the computing device 300 may be a separate device (e.g., a mobile terminal device) from the device 100 and the device 200, and the computing device 300, the device 100, and the device 200 may be in direct communication with the server. Alternatively, the computing device 300 may be in direct communication with the server and the device 100 and the device 200 may be in communication with the server via the computing device 300. It should also be noted that the computing device 300 is illustrative only and does not exclude the possibility of another process-or controller-based system being used in or with any of the aforementioned aspects of the device 100 or the device 200.

In one aspect, the computing device 300 may include one or more hardware and/or software components configured to execute software programs, such as software for obtaining, storing, processing, and analyzing signals, data, or both. For example, the computing device 300 may include one or more hardware components such as, for example, a processor 305, a random-access memory (RAM) 310, a read-only memory (ROM) 320, a storage 330, a database 340, one or more input/output (I/O) modules 350, an interface 360, and one or more sensors 370.

Alternatively, and/or additionally, the computing device 300 may include one or more software components such as, for example, a computer-readable medium including computer-executable instructions for performing techniques or implement functions of tools consistent with certain disclosed embodiments. It is contemplated that one or more of the hardware components listed above may be implemented using software. For example, the storage 330 may include a software partition associated with one or more other hardware components of the computing device 300. The computing device 300 may include additional, fewer, and/or different components than those listed above. It is understood that the components listed above are illustrative only and not intended to be limiting or exclude suitable alternatives or additional components.

The processor 305 may include one or more processors, each configured to execute instructions and process data to perform one or more functions associated with the computing device 300. The term “processor,” as generally used herein, refers to any logic processing unit, such as one or more central processing units (CPUs), digital signal processors (DSPs), application specific integrated circuits (ASICs), field programmable gate arrays (FPGAs), and similar devices. As illustrated in FIG. 3, the processor 305 may be communicatively coupled to the RAM 310, the ROM 320, the storage 330, the database 340, the I/O module 350, the interface 360, and the one or more sensors 370. The processor 305 may be configured to execute sequences of computer program instructions to perform various processes, which will be described in detail below. The computer program instructions may be loaded into the RAM 310 for execution by the processor 305.

The RAM 310 and the ROM 320 may each include one or more devices for storing information associated with an operation of the computing device 300 and/or the processor 305. For example, the ROM 320, may include a memory device configured to access and store information associated with the computing device 300, including information for identifying, initializing, and monitoring the operation of one or more components and subsystems of the computing device 300. The RAM 310 may include a memory device for storing data associated with one or more operations of the processor 305. For example, the ROM 320 may load instructions into the RAM 310 for execution by the processor 305.

The storage 330 may include any type of storage device configured to store information that the processor 305 may use to perform processes consistent with the disclosed examples.

The database 340 may include one or more software and/or hardware components that cooperate to store, organize, filter, and/or arrange data used by the computing device 300 and/or the processor 305. For example, the database 340 may include user profile information, historical activity, and user-specific information (e.g., historical information pertaining to the mental health of a specific user), physiological parameter information, predetermined menu/display options, and other user preferences. Alternatively, the database 340 may store additional and/or different information. For example, the database 340 may include information to establish a machine learning model such as a large language model (LLM) that can receive inputs from the I/O module 350 or the one or more sensors 370.

The I/O module 350 may include one or more components configured to communicate information with a user associated with the computing device 300. For example, the I/O module 350 may include one or more buttons, switches, or touchscreens to allow a user to input parameters associated with the computing device 300. The I/O module 350 may also include a display including a graphical user interface (GUI) and/or one or more light sources for outputting information to the user. The I/O module 350 may also include one or more communication channels for connecting the computing device 300 to one or more secondary or peripheral devices such as, for example, a desktop computer, a laptop, a tablet, a smart phone, a flash drive, or a printer, to allow a user to input data to or output data from the computing device 300.

The interface 360 may include one or more components configured to transmit and receive data via a communication network, such as the internet, a local area network, a workstation peer-to-peer network, a direct link network, a wireless network, or any other suitable communication channel. For example, the interface 360 may include one or more modulators, demodulators, multiplexers, demultiplexers, network communication devices, wireless devices, antennas, modems, and any other type of device configured to enable data communication via a communication network.

The computing device 300 may further include the one or more sensors 370. In one example, the one or more sensors 370 may include one or more of an image sensor 380 and/or other sensors 390, such as an accelerometer, an optical sensor, an acoustical sensor, an ambient light sensor, a pressure sensor, a contact sensor, an electromagnet sensor, an ECG electrode, an EEG electrode, an EOG electrode, an EMG electrode, and/or a bio impedance sensor, etc. It should be noted that these sensors are only illustrative of a few possibilities and the one or more sensors 370 may include alternative or additional sensors suitable for use in the device 100 or the device 200. It should also be noted that although one or more sensors are described collectively as the one or more sensors 370, any one or more sensors or sensor units within the device 100 or the device 200 may operate independently of any one or more other sensors. Moreover, in addition to collecting, transmitting, and receiving signals or information to and from the one or more sensors 370 at the processor 305, any of the one or more sensor units of the one or more sensors 370 may be configured to collect, transmit, or receive signals or information to and from other components or modules of the computing device 300, including but not limited to the database 340, the I/O module 350, or the interface 360.

As described above with respect to FIGS. 1-2B, the accelerometer can be used to detect large-scale motions of a subject indicative of physical activity (e.g., steps, running, walking swimming, etc.) The same accelerometer can be used to determine the onset of a sleep period through the detection of a lack of motion. The acoustical sensor can be used to detect and monitor heart rate. However, in case the sensitivity of the acoustical sensor that detects heart rate is not enough to detect a relatively slow heart rate during sleeping, in one example, upon determining that the subject is engaged in sleep, the sensitivity of the acoustical sensor can be reconfigured to detect a significantly lower heart rate. Alternatively, one or more acoustical sensors can be dedicated to, and configured for, detecting relatively slow heart rate during sleeping while one or more other acoustical sensors are used to detect a regular heart rate during physical activity.

As described above, a person skilled in the art will note that all or a portion of the aspects of the disclosure described herein can be implemented using a general-purpose computer/processor with a computer program that, when executed, carries out any of the respective techniques, algorithms, and/or instructions described herein. In addition, or alternatively, for example, a special-purpose computer/processor, which can contain specialized hardware for carrying out any of the techniques, algorithms, or instructions described herein, can be utilized.

Similarly, all or a portion of the aspects of the disclosure described herein can be implemented by the device 100 or the device 200 (e.g., by the processor 305 when the computing device 300 is incorporated into the device 100 or the device 200), by a server in communication with the device 100 and/or the computing device 300, or both. Additionally, all or a portion of the aspects of the disclosure described herein (e.g., steps, procedures, processes, etc.) may be performed by the device 100, the device 200, or a secondary companion device (e.g., a mobile terminal, a client device, other remote device, etc.). For example, a portion of the steps or procedures described herein may be performed by the aforementioned server while another portion of the steps or procedures may be performed by the secondary companion device.

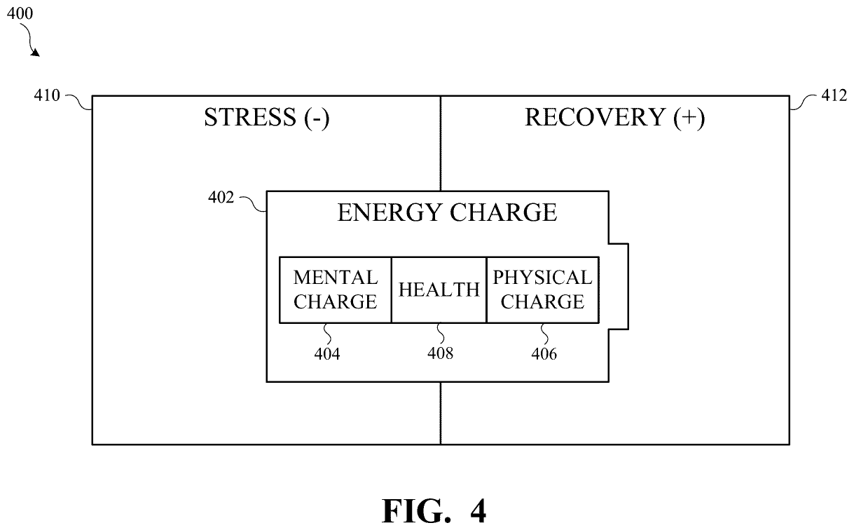

FIG. 4 is a block diagram 400 depicting components that may be utilized to determine an energy charge 402 of a user in accordance with the present teachings. As discussed above, the energy charge 402 of the user may be determined and/or provided as an energy charge value that corresponds to an energy level of the user, which may be caused by, correlated to, associated with, or an indication of the user's physical and/or mental ability to partake in and/or complete a given activity. The activity may be any physical activity (e.g., exercise, physical chores, physical tasks, etc.), mental activity (e.g., work-related tasks, reading, other mental tasks, etc.), or a combination thereof. It should be noted that the components described herein with respect to FIG. 4 are intended as illustrative examples only and that additional or alternative components may also be implemented in accordance with the present teachings.

As discussed further herein, the energy charge 402 of the user (e.g., the energy charge value of the user) may be or may provide an indicator of the user's physical ability and/or mental capacity at a given point in time. That is, the energy charge 402 of the user may provide personalized, real-time information to the user based on the physiological functioning and health-related behaviors of the user (e.g., exercise, rest, sleep, etc.) While the energy charge 402 of the user may be determined and provided to the user as a value or a score, additional information associated with the energy charge 402 of the user may also be provided to the user. For example, the additional information may include guidance (e.g., instructions, recommendations, or information associated with the energy charge 402 of the user determined), information associated with one or more of the components utilized to determine the energy charge 402 of the user (e.g., one or more of the components shown in FIG. 4 and discussed in further detail below), or both.

Turning back to FIG. 4, the energy charge 402 of the user may be determined based on one or more components, whereby the one or more components may be considered sub-components, sub-evaluations, sub-determinations, or the like, of the energy charge 402 of the user. By way of example, the one or more components may each be or may each include a value (e.g., a score), and the values of the one or more components may be utilized to determine the energy charge of the user. As such, the one or more components of the energy charge 402 of the user may be parameter values cumulatively tracked and evaluated to determine the energy charge of the user.

As shown in FIG. 4, the energy charge 402 of the user may be determined based on a mental charge 404 of the user, a physical charge 406 of the user, and the health 408 (e.g., one or more health-related parameters) of the user. The mental charge 404 may be or may include indicators of the mental energy (e.g., the mental energy level) of the user. The physical charge 406 may be or may include indicators of the physical energy (e.g., the physical energy level) of the user. The physical charge 406 may include or take into account physical recovery, or may refer to, or be associated with, physical energy depletion (i.e., consumption) of the user and/or physical energy repletion (i.e., recovery) of the user. Similarly, the mental charge 404 may be or may include or take into account mental recovery, or may refer to, or be associated with, mental energy depletion of the user and/or mental energy repletion of the user.

Thus, the mental charge 404 and/or the physical charge 406 of the user as described herein may also refer to overall recovery of energy depletion and/or energy repletion unless otherwise indicated. For example, physical charge 406 may refer to physical recovery or physical energy recovery of the user and mental charge 404 may refer to mental recovery or mental energy recovery of the user. That is, the energy charge 402 of the user, which may include or otherwise incorporate both the mental charge 404 and the physical charge 406 of the user, may take into account the various activities and/or tasks completed by the user, whereby such activities and/or tasks may contribute to the overall recovery of the user (e.g., as illustrated by the recovery 412 shown in FIG. 4) and/or the overall stress of the user (e.g., as illustrated by the stress 410 shown in FIG. 4). As such, the energy charge 402 of the user, when evaluated and/or determined, may balance the tasks and/or activities completed by the user that may cause mental and/or physical stress (e.g., exercise, physical exertion, anxiety-triggering events, etc.) with the tasks and/or activities completed by the user that may provide mental and/or physical recovery (e.g., sleeping, physical rest, meditation, etc.) to indicate the current energy level of the user at a particular moment in time.

The health 408 of the user may be or may include indicators of health for at least one of breathing health, heart health, and body temperature of the user. The health 408 of the user may be or may include any other indicators of the health of the user, which may be or may include one or more physiological conditions determined based upon various signals obtained by the device 100 or the device 200. That is, the health 408 of the user may include any health-related conditions and/or parameters that further indicate the energy charge 402 of the user when evaluated in conjunction with the mental charge 404 and the physical charge 406 of the user. That is, the mental charge 404, the physical charge 406, and the health 408 of the user may collectively indicate the energy charge 402 of the user. As such, the mental charge 404, the physical charge 406, and the health 408 of the user may, when taken as a whole, indicate whether the user is ready to partake in and/or complete a particular activity or needs additional rest and recovery before such an activity can be done successfully.

The mental charge 404, the physical charge 406, and the health 408 of the user may be determined based on data obtained in any desired manner. By way of example, the mental charge 404, the physical charge 406, the health 408 of the user, or a combination thereof may be determined based on physiological signals (or sensor signals) detected by the device 100 or the device 200 when worn by the user. The physiological signals detected by the device 100 or the device 200 may be associated with one or more physiological conditions of the user, whereby the one or more physiological conditions of the user may be associated with the health 408 of the user. Similarly, the one or more physiological conditions may be further assessed or evaluated to determine the mental charge 404 and/or the physical charge 406 of the user.

In addition to physiological signals detected by the device 100 or the device 200, one or more of the components of the energy charge 402 (e.g., the mental charge 404, the physical charge 406, the health 408, or a combination there) may also, in certain cases, be partially or entirely based on subjective feedback provided by the user. For example, the user, via the device 100, the device 200, or another device, may provide feedback with respect to their subjective feeling of their energy charge (i.e., their subjectively felt energy level). The feedback may be in the form of answers to a questionnaire and/or other data manually input by the user. Such feedback may then be utilized as an additional or alternative metric to those determined based on the physiological signals detected by the device 100 or the device 200. That is, the energy charge 402 (e.g., the energy charge value) of the user may be determined based on objective data (e.g., the physiological signals detected by the device 100 or the device 200), subjective data (e.g., the feedback from the user), or both. As a result, the energy charge 402 may be personalized and more tailored to the user.

By way of example, the energy charge 402 (e.g., the energy charge value) of the user may be based upon both the objective data (e.g., the physiological signals detected by the device 100 or the device 200) and the subjective feedback (e.g., answers to questions provided by the user) such that the energy charge 402 and/or one or more components of the energy charge 402 (e.g., the mental charge 404 and/or the physical charge 406) may be adjusted based upon the user's subjective experience. That is, the user may be prompted to provide answers to questions specifically tailored to one or more of the components of the energy charge 402 to input how the user subjectively feels with respect to the one or more components. For example, the user may provide feedback via the device 100 or the device 200 to input what they currently feel is the mental charge 404 (e.g., their mental energy level) and/or what they currently feel is the physical charge 406 (e.g., their physical energy level). Based on such inputs, the determination (e.g., calculation or estimation) of one or more of the components (e.g., the mental charge 404 and/or the physical charge 406) and/or the energy charge 402 as a whole that is based on the objective data (e.g., the physiological signals detected) may be modified or otherwise adjusted to take into account the user's input.

For example, the physical charge 406 estimated using objective data may indicate the user currently has a higher level of physical energy. However, the user may provide feedback that they are currently feeling physically tired. As a result, the physical charge 406 (e.g., a physical charge value) of the user may be adjusted (e.g., decreased) based upon the user's feedback. Similarly, or additionally, the energy charge 402 (e.g., the energy charge value) may be adjusted to account for the dissimilarities between the physical charge 406 of the user objectively determined and the physical charge 406 of the user subjectively input. It should be noted that a similar process may be completed for any of the components utilized to determine the energy charge 402, such as the mental charge 404.

As discussed above, the energy charge 402 of the user may be determined based on the physical charge 406 of the user. As discussed further below, the physical charge 406 may be determined based on periods of activity and/or rest. The activity and the rest of the user may be based on one or more of the physiological conditions of the user at a given point in time, such as heart rate, blood pressure, body temperature, or other physiological conditions. That is, the health 408 of the user may be utilized to determine the physical charge 406 of the user while also providing additional insight (e.g., metrics) for determining the energy charge 402. For example, a period of activity of the user or a period of rest of the user may be determined at least in part by the aforementioned physiological conditions. The periods of activity and rest of the user may indicate levels (e.g., durations) of physical exertion and physical rest such that the physical charge 406 of the user may be determined. That is, the physical exertion may be considered stress 410 on the user whereas the physical rest may be considered the recovery 412 (e.g., physical recovery) of the user. As such, the physical charge 406—and thus the energy charge 402—may be determined based on a balance between the activity of the user and the rest of the user.

The physical charge 406 may be determined based on at least one of heart rate data obtained from the heart rate sensor and motion data obtained from the motion sensor. For example, the physical charge 406 of the user is determined based on one or more activity metrics and/or one or more heart rate metrics of the user. By way of example, and as discussed in further detail below, the physical charge 406 may be at least partially based on the critical point model of exercise intensity and physical energy reserve. That is, the physical charge 406 may take into account an intensity of an exercise (e.g., the activity of the user). For example, it may be determined that the physical charge 406 of the user has been at least partially consumed when the intensity of the activity (e.g., high, medium, low) is higher than a critical point (e.g., a predefined threshold value for one or more parameters used to determine the intensity of the activity).

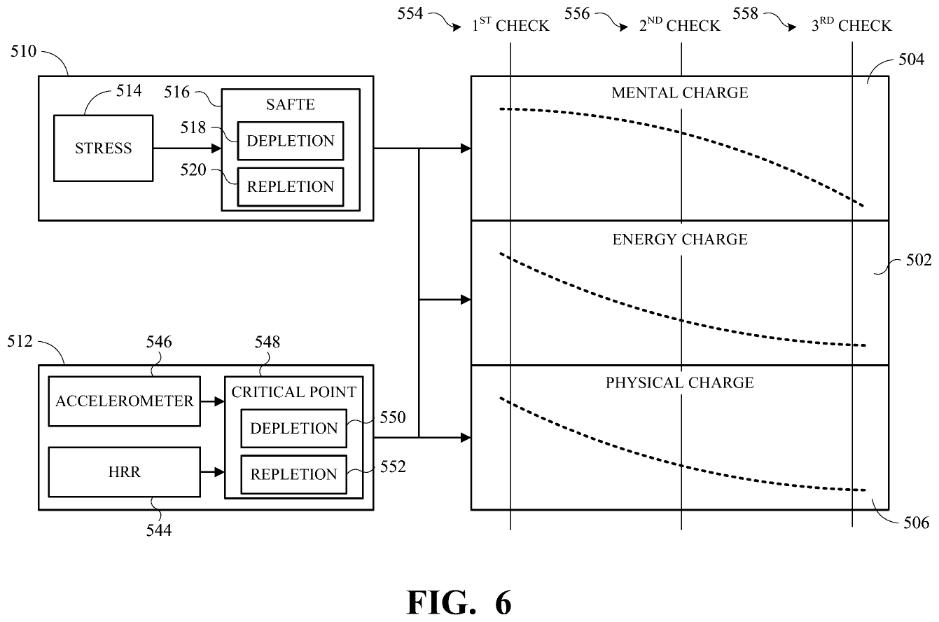

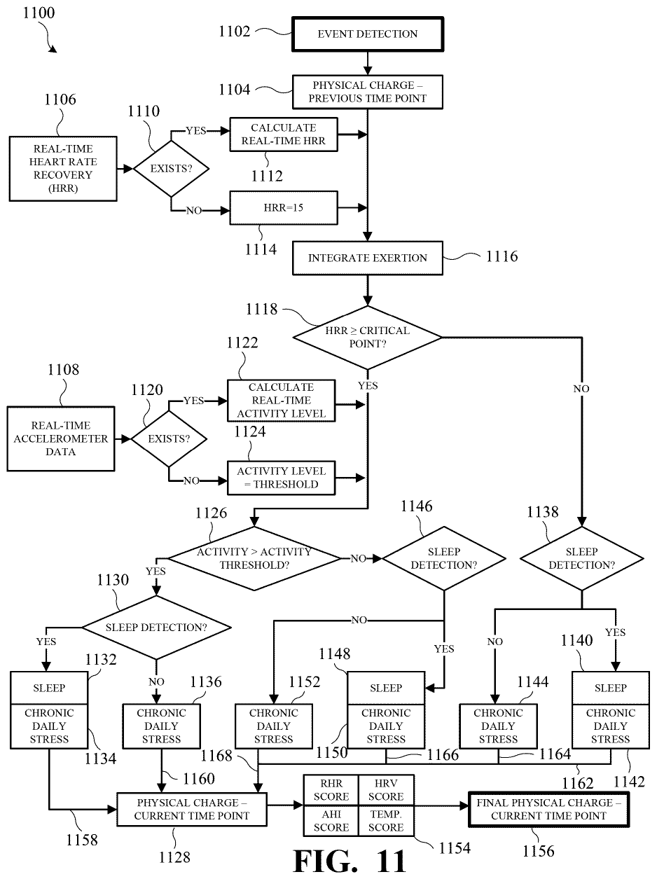

Conversely, it may be determined that the physical charge 406 of the user is taking place when the intensity of the activity is lower than the critical point (e.g., when the physical energy of the user consumed for the activity is lower than the critical point). In other words, the physical charge 406 of the user may be or may include determining whether, based on the periods of activity and the periods of rest, the user is in fact physically recovering or instead expending physical energy (i.e., physical energy consumption). A method of determining the physical charge 406 of the user is discussed in further detail below with respect to FIGS. 6 and 11.

As discussed above, the energy charge 402 of the user may also be determined based on the mental charge 404 of the user. As shown in FIG. 4, and similar to the physical charge 406, the mental charge 404 may be determined based on periods of stress 410 and/or period of recovery 412. The stress 410 and the recovery 412 of the user may be based on one or more of the physiological conditions of the user at a given point in time, such as heart rate, blood pressure, body temperature, or other physiological conditions. For example, a period of stress 410 of the user or a period of recovery 412 of the user may be determined at least in part by the aforementioned physiological conditions. The periods of stress 410 and recovery 412 may indicate levels (e.g., durations) of mental exertion and mental recovery (e.g., mental rest based upon a lack of activity and/or sleep) such that the mental recovery of the user may be determined. That is, the mental charge 404 may be determined based on a balance between the stress 410 of the user and the recovery 412 of the user.

By way of example, and as discussed in further detail below, the mental charge 404 may be at least partially based on the sleep, activity, fatigue, and task effectiveness (SAFTE) model. The SAFTE model may be implemented herein to effectively predict the effects of sleep metrics, circadian rhythms, and workload on mental fatigue (e.g., stress 410) and mental recovery (e.g., recovery 412). That is, a method of determining the mental charge 404 herein may be at least partially based on the SAFTE model and may incorporate various metrics associated with the user, such as but not limited to, circadian rhythm factors and various sleep metrics (e.g., sleep duration, start and end times of a sleep cycle, interruptions in sleep, etc.) Such metrics associated with the user may be determined at least in part by physiological signals detected by the device 100 or the device 200 when worn by the user or other devices monitoring the user.

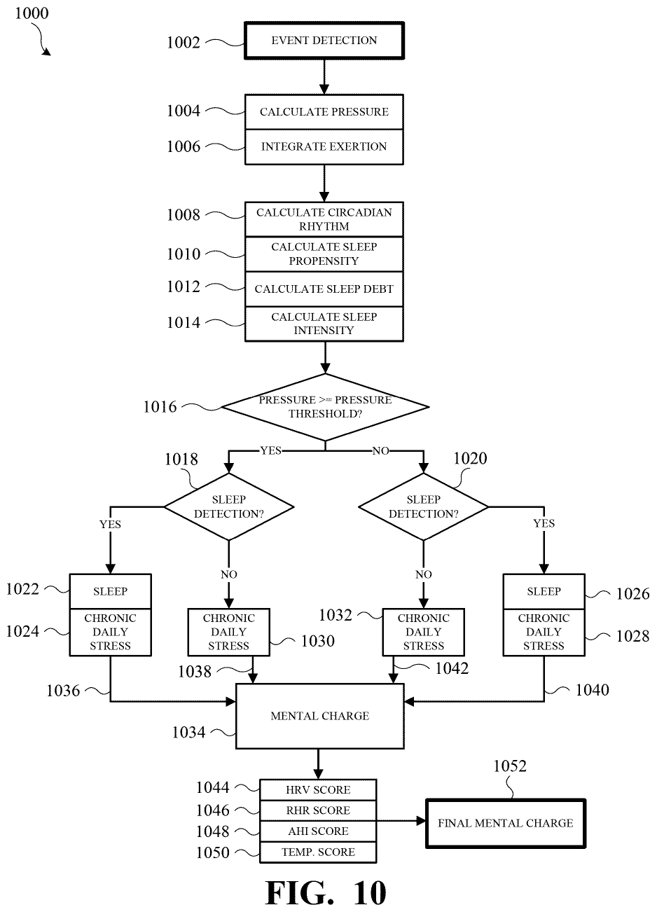

By utilizing the SAFTE model and various other metrics, the method of determining the mental charge 404 may dynamically determine periods of sleep of the user as well as periods of quiet immobility (e.g., meditation) of the user, which may both contribute to the mental charge 404 of the user. That is, the mental charge 404 of the user may be or may include determining whether, based on the periods of stress 410 and periods of recovery 412, the user is in fact mentally recovering or instead expending mental energy (i.e., mental energy consumption). A method of determining the mental charge 404 of the user is discussed in further detail below with respect to FIGS. 6 and 10.

The energy charge 402 (e.g., the energy charge value) of the user may also be determined based on the health 408 of the user, which may include breathing health, heart health, body temperature of the user, other factors, or a combination thereof. Each of these factors may be determined based on one or more parameters. The one or more parameters of each of these factors may be, or may be determined based on, one or more physiological signals captured by the device 100 or the device 200 when worn by the user or another device monitoring the user. That is, the factors utilized to determine the health 408 of the user may be determined based at least partially on physiological conditions of the user, whereby such physiological conditions of the user may be determined based on the one or more physiological signals.

To further illustrate, the breathing health of the user may be associated with breathing of the user and the amount of oxygen in the user's blood. Poor breathing may be an indication of a health issue of the user. By way of example, if the user is sick, it may be difficult for the user to breathe, which may in turn cause the oxygen in the user's blood to drop. Additionally, if the user exhibits sleep disordered breathing (e.g., obstructive sleep apnea) pauses in breathing during sleep may cause the oxygen in the user's blood to drop frequently during the night. Such poor breathing, especially during sleep, may hinder the user's ability to recover physically and/or mentally. Thus, the breathing health may be utilized in conjunction with the physical charge 406 and/or the mental charge 404 determined to more accurately track whether the user physically and/or mentally recovers. Similarly, the breathing health may be utilized to modify the determinations made for the physical charge 406 and/or the mental charge 404 to more accurately reflect any breathing health conditions exhibited by the user.

The breathing health of the user may be determined (e.g., measured or estimated) based on fluctuations in oxygen saturation and/or the apnea-hypopnea index (AHI). For example, fluctuations in oxygen saturation and/or the AHI may be determined (e.g., extracted) from a photopolythesogram (PPG) signal detected by the device 100 when worn by the user. Using domain knowledge boundaries (e.g., predefined boundaries for values of oxygen saturation and/or the AHI), a level of oxygen saturation and AHI may be determined. It should be noted that the above methodology to determine the breathing health is intended as an example and other methods of determining the breathing health of the user may be possible.

The heart health of the user may be associated with functionality of the heart of the user. For example, the heart health may be associated with a heart rate of a user, whereby the heart rate may be an indicator of how fast or slow the user's heart is beating. The heart rate and variability thereof may be indicators of how healthy the user's heart may be and how well the user's body may adapt to physical and/or mental changes. Thus, the heart health may be utilized in conjunction with the physical charge 406 and/or the mental charge 404 determined to more accurately track whether the user physically and/or mentally recovers. Similarly, the heart health may be utilized to modify the determinations made for the physical charge 406 and/or the mental charge 404 to more accurately reflect any heart conditions exhibited by the user.

The heart health of the user may be determined (e.g., measured or estimated) based on a resting heart rate (RHR) and/or heart rate variability (HRV) of the user. The RHR may measure or be a measurement of the heart rate of the use while at rest and/or during sleep. A lower RHR may be an indication of better heart health of the user while a higher RHR may indicate poorer heart health. Moreover, HRV may measure or be a measurement of how well the user's body (including the heart of the user) can adapt to physical and/or mental changes (e.g., physical and/or mental exertion or stress). A higher HRV may be an indication of less stress and better overall health of the user while a lower HRV may indicate higher stress and poorer over health of the user. Using domain knowledge boundaries (e.g., predefined boundaries for values of the RHR and/or HRV) alongside the user's historical data and baselines, the RHR and HRV may be determined. It should be noted that other factors other than the RHR and HRV of the user may be utilized to determine the heart health of the user.

The body temperature of the user fluctuates (e.g., increases and/or decreases) throughout the course of the day based upon (e.g., in response to) the environment, meals, exercise, and other factors experienced by the user. For example, the user's brain may regulate their body temperate in response to any conditions. A body temperature that is higher than normal may indicate oncoming illness or significant stress for the user, suggesting that the user may need additional time to rest and/or recover during the day.

The body temperature of the user may be determined (e.g., measured or estimated) based on a skin temperature of the user and/or a core temperature (i.e., core body temperature) of the user. For example, fluctuations in the core temperature may be estimated based on measuring the skin temperature of the user, such as during sleep, and the core temperature may be determined based on the skin temperature measurement and associated heart rate measurement. If the fluctuation of the core temperature estimates exceeds about 1° C. or another predefined threshold, it may indicate a potential deviation from a normal temperature range of the user. Such deviation may be a sign of oncoming illness or significant stress.

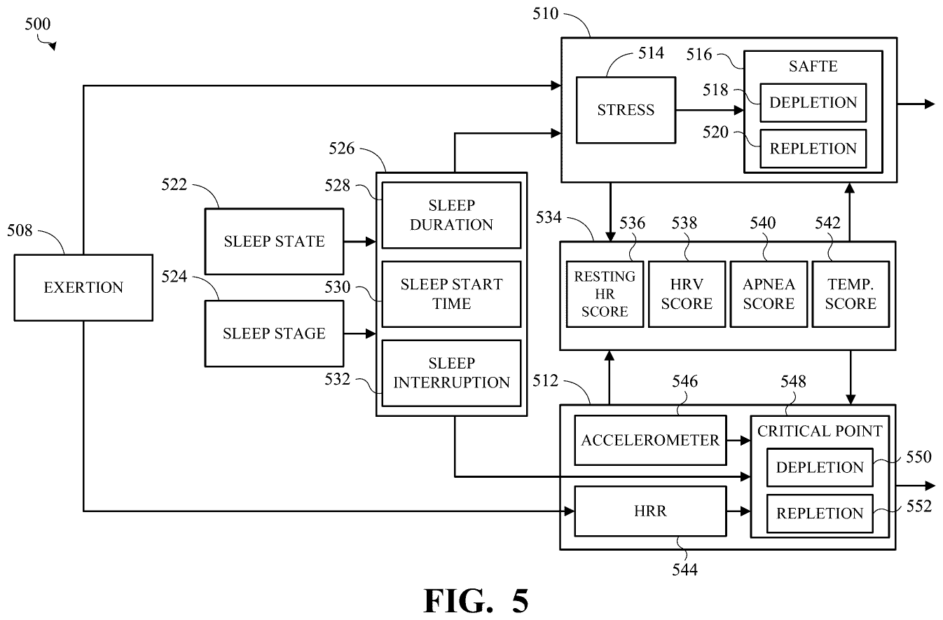

To further illustrate the energy charge 402 discussed above, FIG. 5 illustrates a flow diagram of an example of a method 500 for dynamically determining the energy charge 402 of the user. FIG. 6 is a continuation of the method 500 shown in FIG. 5. As discussed herein, the energy charge 402 may correspond to an overall energy level of the user, such as the energy charge 502 shown in FIG. 6. The energy charge 502 (e.g., the overall energy charge of the user) may be based on, or take into account, a mental charge 504 of the user and/or a physical charge 506 of the user.

The energy charge 502 may be determined based on periods of activity and periods of rest of the user, whereby the user may alternate between the periods of activity and the periods of rest. During such periods of activity and periods of rest, the method 500 may evaluate or otherwise monitor both the mental charge 504 (e.g., the mental energy level) and the physical charge 506 (e.g., the physical energy level) of the user. Such an evaluation or monitoring may be initiated based upon any desired time interval or activity.

By way of example, the method 500 may start dynamically determining the energy charge 502 based upon exertion 508 of the user. The exertion 508 may be an activity or task the user begins and/or completes. That is, the method 500 may begin to determine the energy charge 502 when the user begins the activity or task (e.g., the exertion 508) and/or when the user completes the activity of task (e.g., the exertion 508). The exertion 508 may be any activity or task, such as a physical exertion (e.g., exercise or other types of physical movement) or a mental exertion (e.g., reading, a mentally stressful situation, etc.). The method 500 may determine when the user is partaking in the exertion 508 based upon monitoring the user via the device 100. For example, the device 100 or the device 200 may monitor one or more physiological signals that may correspond to physical and/or mental exertion of the user. As such, the method 500 may monitor any exertion 508 of the user to more accurately determine the energy charge 502 of the user.

Based upon determining that the exertion 508 is occurring and/or has occurred, the method 500 may determine a mental energy 510 of the user and a physical energy 512 of the user. As shown in FIG. 6, the mental energy 510 may correspond to the mental charge 504 of the user and/or the mental charge 504 may be based upon the mental energy 510. Similarly, the physical energy 512 may correspond to the physical charge 506 of the user and/or the physical charge 506 may be based upon the physical energy 512.

The mental energy 510 may further evaluate the exertion 508 based upon one or more components to determine the mental energy 510 of the exertion 508. That is, the mental energy 510 may further evaluate the exertion 508 to determine the overall mental energy exerted by the user during the exertion 508. For example, to determine the mental energy 510 of the user, the method 500 may determine a stress 514 of the user. The stress 514 may be or may correspond to a level of pressure mentally felt by the user when partaking in and/or completing the exertion 508. As such, the stress 514 may correspond to a level of mental stress (e.g., low, medium, high) of the user. For example, the stress 514 may indicate that the user has a high level of stress, which may be a further indication of a higher level of mental energy 510, thereby resulting in a greater depletion (i.e., use) of the energy charge 502 of the user.

The stress 514 of the user may be based on one or more of the physiological conditions of the user at a given point in time, such as heart rate, blood pressure, body temperature, or other physiological conditions. Such physiological conditions may be based on one or more physiological signals received by the device 100 or the device 200 when worn by the user. For example, in a situation where the stress 514 of the user is heightened, the user may exhibit an increased heart rate, blood pressure, body temperature, electrodermal activity, or a combination thereof. Such increases in one or more physiological conditions may be detected by the device 100 when worn by the user.

The mental energy 510 may also be at least partially based on the SAFTE model 516, which may be at least partially based upon the stress 514 determined and/or may be determined in addition to the stress 514 of the user. As discussed above, the SAFTE model 516 may be implemented to effectively predict the effects of sleep metrics, circadian rhythms, and workload on mental fatigue and mental recovery. Mental fatigue, which may occur during higher levels of stress (e.g., the stress 514) and/or exertion, may correspond to mental depletion 518, which may indicate a decrease in the mental charge 504 of the user. Conversely, mental recovery, which may occur during lower levels of stress (e.g., the stress 514) or where no stress (e.g., the stress 514) of the user is detected, such as during sleep or rest, may correspond to mental repletion 520, which may indicate an increase in the mental charge 504 of the user. By utilizing the SAFTE model 516 and determining the stress 514 of the user, determining the mental energy 510 may be dynamically influenced by periods of sleep and/or quite immobility of the user as well as periods of increased exertion.

By way of example, to accurately utilize the SAFTE model 516, the method 500 may include dynamically determining a sleep state 522 and a sleep stage 524 of the user. That is, the method 500 may include, in addition to determining that the exertion 508 is or has occurred, determining whether the user is sleeping based upon identifying the sleep state 522 and the sleep stage 524 of the user. The sleep state 522 may correspond to an overall behavioral and/or physiological condition of the user to identify whether the user is asleep or in a transitional phase. For example, the sleep state 522 may identify whether the user is in one or more of the following wakefulness/sleep states: wake, active wake, quiet wake, sleep, sleep latency, sleep inertia, and wakefulness after sleep onset. The sleep stage 524 may identify whether the user is in one or more of the following sleep states: wake, NREM sleep, REM sleep, shallow/light sleep, drowsiness, and microsleep episodes. Furthermore, the sleep stage 524 may also correspond to a specific physiological pattern or depth of sleep observed during a typical sleep cycle. For example, the sleep stage 524 may identify whether the user is in one or more of the following sleep stages: N1 (e.g., Stage 1, which may correspond to light sleep), N2 (e.g., Stage 2, which may correspond to moderate sleep), N3 (e.g., Stage 3, which may correspond to deep sleep), and REM (e.g., Rapid Eye Movement, which may correspond to dreaming and brain activity similar to wakefulness). The sleep state 522 and/or the sleep stage 524 may be determined based upon the physiological conditions of the user (e.g., based upon the physiological signals obtained by the device 100 when worn by the user).

The method 500 may further include determining additional sleep components 526 that may be related to the sleep state 522 and/or the sleep stage 524. For example, the additional sleep components 526 may include one or more of a sleep duration 528 (e.g., a duration of time in which the user is asleep or in a particular type of sleep state 522 and/or sleep stage 524), a sleep start time 530, and a sleep interruption 532 (e.g., whether and/or how often the user's sleep is interrupted). Such components may also be determined based upon the physiological conditions of the user. As such, the method 500 may include a detailed evaluation of various sleep metrics of the user, analyzed based on both time and value (e.g., assigned scores associated with the various sleep metrics) to more accurately determine the mental energy 510 of the user.

To even further improve the accuracy of the mental energy 510 determined, the method 500 may also integrate health metrics 534 into the evaluation. By way of example, the method 500 may include determining one or more of a resting heart rate (HR) score 536, a HR variability (HRV) score 538, an apnea score 540 (i.e., a sleep apnea score), and a temperature score 542. Such scores may be based on evaluating the values of the health metrics 534 that are based on the physiological conditions of the user.

By way of example, the temperature score 542 may be determined by evaluating the actual body temperature of the user. For example, the actual body temperature of the user may be obtained via one or more physiological signals obtained by the device 100 when worn by the user. The body temperature of the user may then be evaluated, such as based upon one or more temperature thresholds and/or temperature ranges to determine whether the body temperature is a healthy temperature or an unhealthy temperature (i.e., a body temperature indicating that the user may be sick). For example, if the body temperature of the user is greater than 37.5° C., which may be an indication that the user has a low-grade fever, the temperature score 542 may be higher than if the user had a body temperature that is less than 37.5° C. (i.e., indicating a normal body temperature). As such, a temperature score 542 may indicate a greater level of energy depletion of the user. Though the temperature score 542 was discussed above, a similar evaluation may be done for any one of the health metrics 534 discussed above.

As discussed above, in addition to determining the mental energy 510 of the user, the method 500 may also include determining the physical energy 512 of the user. The physical energy 512 may further evaluate the exertion 508 based upon one or more components to determine the physical energy 512 of the exertion 508. That is, the physical energy 512 may further evaluate the exertion 508 to determine the overall physical energy consumption by the user during the exertion 508. For example, the physical energy 512 of the user may be determined and/or based at least partially upon heart rate data, such as a heart rate reserve (HRR) 544 of the user and/or accelerometer data 546 (e.g., accelerometer data obtained by the device 100 or the device 200). The HRR 544 of the user may be utilized to assess the physical fitness (e.g., the cardiovascular fitness) of the user to determine the user's real-time exercise intensity or physiological exercise power based upon a particular activity, such as the exertion 508. As such, the HRR 544 and the accelerometer data 546 may be used to assess the activity level of the user (e.g., the activity level of the user during the exertion 508) and thereby determine the physical energy 512 of the user.

By way of example, the HRR 544 and the accelerometer data 546 may be used in conjunction with a critical point (CP) 548. The CP 548 may be any predefined threshold value that is compared to the HRR 544 to determine whether physical depletion 550 or physical repletion 552 occurs. For example, if the HRR 544 is greater than or equal to the CP 548 (i.e., greater than or equal to the predefined threshold value of the CP 548), then physical depletion 550 may occur, indicating the physical energy 512 of the user may be higher, and thus the physical charge 506 of the user may be depleted. Conversely, if the HRR 544 is less than the CP 548 (i.e., less than or equal to the predefined threshold value of the CP 548), then physical repletion 552 may occur, indicating the physical energy 512 of the user is being replenished, and thus the physical charge 506 of the user may be increased (e.g., “recharged”).

It should be noted that the additional sleep components 526 (e.g., sleep duration 528, sleep start time 530, and sleep interruption 532) may be utilized to determine the physical energy 512 of the user in a similar manner to the techniques described above with respect to utilized the additional sleep components 526 to more accurately determine the mental energy 510. For example, if a user is sleeping (e.g., the sleep state 522 is determined to be anything other than wake), the sleep stage 524 and the one or more of the additional sleep components 526 may be determined to evaluate whether the sleep of the user impacts the physical energy 512 of the user. By way of example, if the user is restfully sleeping, the physical energy 512 determined may indicate that physical repletion 552 occurs, thereby indicating that the physical charge 506 of the user may be increased.

The health metrics 534 may also be used in a similar manner to improve the accuracy of the physical energy 512 determined. That is, one or more of the RHR score 536, the HRV score 538, the apnea score 540, and the temperature score 542 may be incorporated into the determination of the physical energy 512 to more precisely determine the physical energy 512 of the user. Such implementations may be similar to those discussed above with respect to utilizing the health metrics 534 to improve the accuracy of the mental energy 510 determined. Moreover, the mental energy 510 and/or the physical energy 512 determined may aid in more accurately determining one or more of the values of the health metrics 534, which may in turn even further improve the accuracy of the energy charge 502 determined.

As discussed above, the exertion 508 of the user may be monitored (e.g., monitored by the device 100 or another electronic device), whereby the method 500 may evaluate the exertion 508 of the user to determine the energy charge 502 of the user. That is, the exertion 508 of the user may be evaluated to determine whether the energy charge 502 of the user may increase or decrease. As such, the energy charge 502 of the user may be dynamically determined in response to the exertion 508 of the user.

Additionally, the energy charge 502 of the user may be dynamically determined based on a desired schedule. For example, the method 500 may be performed on a desired time interval such that one or more “checks” may occur over the course of a day, week, or month. By way of example, FIG. 6 illustrates that a first check 554, a second check 556, and a third check 558 may be completed over the course of a particular day. For each check, the method 500 may be performed, whether or not the exertion 508 occurs, to determine one or more of the energy charge 502, the mental charge 504, and the physical charge 506 of the user at the desired time.

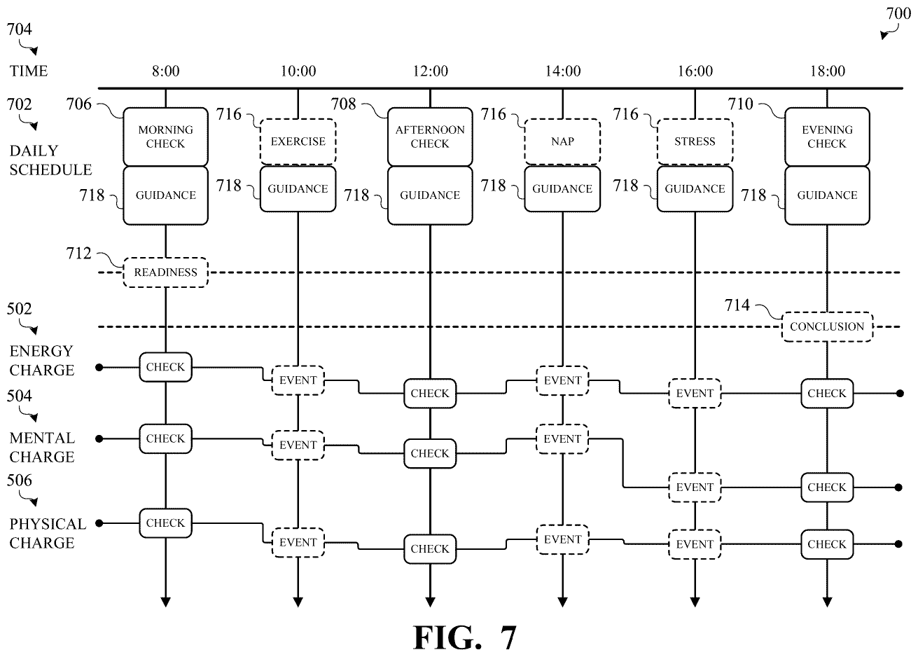

To further illustrate, FIG. 7 illustrates an example of a plan 700 for dynamically determining the energy charge 502 of the user. As shown in FIG. 7, the plan 700 may include a daily schedule 702 to evaluate and dynamically determine the energy charge 502, the mental charge 504, and the physical charge 506 of the user. As discussed above, the mental charge 504 and the physical charge 506 may be components (e.g., subcomponents) of the energy charge 502 and may be used to determine the energy charge 502, which may correspond to the overall energy level of the user.

The daily schedule 702 may include one or more checks, one or more activities, other actions, or a combination thereof over a period of time 704. While a 10-hour period of time 704 is illustrated in FIG. 7 (e.g., from 8:00 AM to 6:00 PM), any duration of time 704 for the daily schedule 702 may be possible. For example, it is envisioned that the daily schedule 702 may reflect any particular day and thus may encompass a 24-hour period of time 704. Moreover, the daily schedule 702 may vary for any particular day to reflect the checks, activities, other actions, or a combination thereof for that particular day. As such, FIG. 7 illustrates only an example of a daily schedule 702.

The daily schedule 702 may include a morning check 706, an afternoon check 708, and an evening check 710. The aforementioned checks may be a point in time 704 in which the energy charge 502 of the user may be determined (e.g., using the method 500 discussed above), and as such, the mental charge 504 and/or the physical charge 506 may also be determined (e.g., using the method 500 discussed above). As such, for each of the morning check 706, the afternoon check 708, and the evening check 710, the energy charge 502, the mental charge 504, and the physical charge 506 may be checked, as illustrated by the boxes in FIG. 7 labeled with “CHECK”.

The morning check 706 may be scheduled as an initial determination of the energy charge 502 of the user upon waking or shortly thereafter. For example, the morning check 706 may align, or take into account, a readiness 712 of the user. The readiness 712 may be or may include determining a readiness score of the user that is associated with a readiness of the user to partake in one or more activities over the course of the day (e.g., based on the daily schedule 702). For example, the readiness 712 (i.e., the readiness score) may be based on historical estimations for one or more energy charge components (e.g., the mental charge 504 and/or the physical charge 506). The historical estimation for the one or more energy charge components may be based on historical sensor or physiological data (e.g., physiological signals obtained by the device 100) that is tracked on one or more timescales (e.g., at least one of a daily timescale, a weekly timescale, and a monthly timescale). As such, the readiness 712 (i.e., the readiness score) may represent an initial energy charge (the energy charge 502) of the user for a particular day. Thus, the energy charge 502, such as during the morning check 706, may be based on the readiness 712 (i.e., based on the readiness score). The readiness 712 (i.e., the readiness score) may be determined based upon the teachings of U.S. application Ser. No. 18/820,790, the contents of which are incorporated herein in their entirety for all purposes.

The afternoon check 708 may be scheduled as a midday determination of the energy charge 502 of the user. For example, the afternoon check 708 may be scheduled at 12:00 PM midday to determine energy charge 502 of the user approximately halfway through the day (e.g., halfway through their time awake for the day).

The evening check 710 may be scheduled as a final determination of the energy charge 502 of the user. For example, the evening check 710 may be completed just prior to the user sleeping for the night. As such, the evening check 710 may be considered a conclusion 714 of the energy charge 502 for the day. As such, the conclusion 714 may in turn correlate to the readiness 712 and/or the energy charge 502 of the user at the morning check 706 of the following day. That is, the conclusion 714 may reflect the energy charge 502 of the user prior to sleeping, whereby the morning check 706 of the subsequent day may determine that energy charge 502 is similar to the energy charge 502 of the user at the evening check 710 of the prior evening, while taking into account an impact to the energy charge 502 from the sleep of the user.

The conclusion 714 may be or may include the energy charge 502, the mental charge 504, the physical charge 506, or a combination thereof. The conclusion 714 may also be output to the user as a summary of the energy charge 502 of the user. The conclusion 714 may also reflect the one or more activities through the daily schedule 702, such as the activities 716. The activities 716 may correspond to or may be the exertion 508 discussed above with respect to the method 500 shown in FIG. 5. For example, the activities 716 may be an exercise, a nap, a stress-inducing activity, another activity, or a combination thereof. As such, responsive to the activities 716 commencing and/or occurring, the energy charge 502 of the user may be determined based upon the method 500 described above. That is, the activities 716 may trigger additional checks of the energy charge 502, the mental charge 504, and the physical charge 506 of the user in addition to the morning check 706, the afternoon check 708, and the evening check 710, as illustrated by the dashed line boxes in FIG. 7 labeled with “EVENT”.

Additionally, or alternatively, the activities 716 be or may include a recovery activity (e.g., a recovery session) engaged in by the user immediately or soon after an activity that may require physical and/or mental exertion by the user (e.g., exercise) to enhance muscle recovery and stimulate growth. As such, when the activities 716 include or are a recovery activity, such activities may positively impact the energy charge 502 of the user. For example, if the user engages in a recovery activity (e.g., a sauna, ice bath, meditation, hyperbaric chamber, etc.), their energy expenditure (e.g., expenditure of the energy charge 502) will significantly decrease and the energy charge 502 may be maintained. As such, the plan 700 may include one or more recovery activities, which may be part of the activities 716, which may be taken into account when determining the energy charge 502 of the user.

By way of example, if a check (e.g., determination) of the energy charge 502 is triggered by one of the activities 716, such as by the user exercising (e.g., as determined by the device 100, the device 200, or another device), the method 500 may be unable to determine whether a portion of the activity includes a recovery activity based upon the one or more physiological conditions of the user. As such, the user may be prompted (e.g., via the device 100, the device 200, or another device) to input whether they have completed a recovery activity and/or plan on completing a recovery activity, such as those described above. If a recovery activity is confirmed, the recovery activity may be integrated into one or more of the mechanisms utilized to determine the mental charge 504 and/or the physical charge 506, as described further below with respect to FIGS. 10 and 11.

By way of example, recovery activities of the user may function to minimize the expenditure of the energy charge 902 of the user. For example, if the user is awake, expenditure of the energy charge 902 of the user may typically happen at a specific rate based on a level of mental stress and physical activity of the user. Conversely, during a recovery activity (e.g., a recovery session), the expenditure of the energy charge 902 may be minimized to a basal (e.g., nominal) rate. Similarly, if the user is awake and is partaking in an activity that may replenish the energy charge 902 based upon the one or more physiological signals obtained by the device 100 or the device 200, such replenishment may be further accentuated (e.g., increased) during a recovery activity of the user. Thus, recovery activities may provide an additional means to more accurately determine the energy charge 902 of the user.

It should be noted that any check, whether scheduled or triggered by the activities 716, may also include guidance 718. For example, for one or more of the aforementioned checks, the energy charge 502, the mental charge 504, the physical charge 506, or a combination thereof may be provided to the user (e.g., displayed to the user on a display of the device 100, the display unit 212 of the device 200, or a display of another device). The charge(s) may be provided to the user the guidance 718, which may be instructions, recommendations, information, or a combination thereof that may be associated with the charge(s). The guidance 718 may be expert-curated guidance to accompany the different variations of the charge(s). The guidance 718 may be or may include information associated with the charge(s), instructions on how to address an underlying situation associated with the charge(s), recommendations (e.g., recommended activities or tasks), or a combination thereof.

By way of example, the guidance 718 may be a descriptive analysis of one or more of the energy charge 502, the mental charge 504, and the physical charge 506, which may be provided to the user. In such a case, the descriptive analysis may also describe one or more factors utilized to determine one or more of the energy charge 502, the mental charge 504, and the physical charge 506 (e.g., one or more of the components shown in FIGS. 5-6 and described above). Additionally, or alternatively, the guidance 718 may be an assessment of the current charge of the user (e.g., one or more of the current energy charge 502, the mental charge 504, and the physical charge 506). For example, the assessment may include a determination of whether the current charge of the user is sufficient based on time duration, one or more potential upcoming activities, the current time of 704 of the day, or a combination thereof. That is, the assessment may evaluate whether the user's current charge (e.g., one or more of the current energy charge 502, the mental charge 504, and the physical charge 506) is sufficient to complete an upcoming task or activity and provide such an assessment to the user. Furthermore, the guidance 718 may be or may include one or more recommendations that are provided to the user to recommend one or more activities—which may include one or more recovery recommendations (e.g., recovery activities, such as rest, meditation, etc.)—based on the user's current charge and/or one or more factors utilized to determine the current charge.

Additionally, the guidance 718 may be in the form of an interactive conversation (e.g., dialogue) with the user. For example, a chatbot may utilize large language models (LLMs) to cure domain knowledge bases and metrics that cover all the charge(s) and the components thereof that are utilized to determine the charge(s). The user may then use natural language (e.g., via voice or text) to ask questions about the domain knowledge or readings related to the components and/or the charge(s), thereby providing further personalization to the user.

It can be seen from FIG. 7 that the plan 700 may include several fluctuations in the energy charge 502, the mental charge 504, and the physical charge 506 of the user, while those charges may not necessarily fluctuate in the same manner.

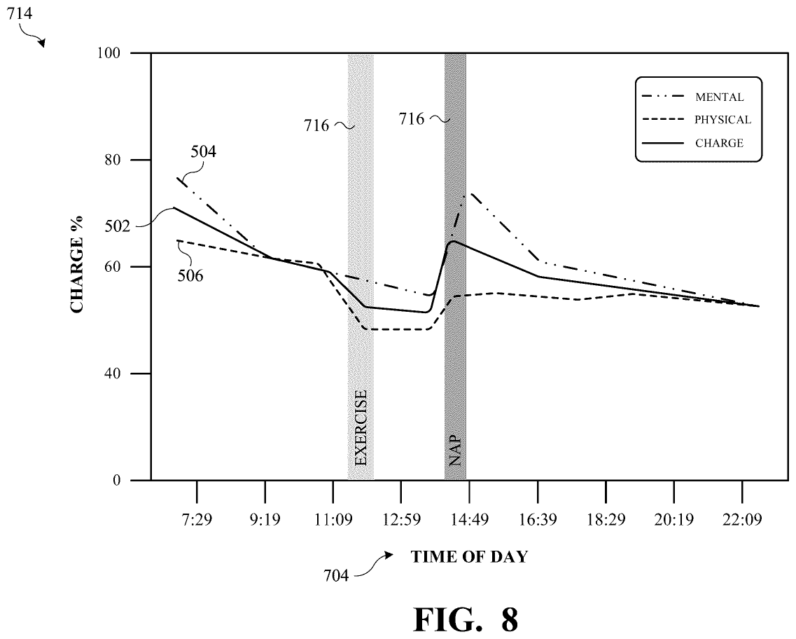

To clearly illustrate such fluctuations to the user, FIG. 8 illustrates an example of the conclusion 714, which may be visually provided to the user to help the user review their energy levels throughout the day. The conclusion 714 may be provided to the user in any desired manner and/or format.

By way of example, the conclusion 714 may graphically display the energy charge 502, the mental charge 504, and the physical charge 506 over the period of time 704 shown in the plan 700. The graphical display may also include the activities 716 that occurred during a particular day. It should be noted that the conclusion 714 represents an example of a graphical display and does not necessarily reflect each of the items shown in the plan 700 to further simplify the example.

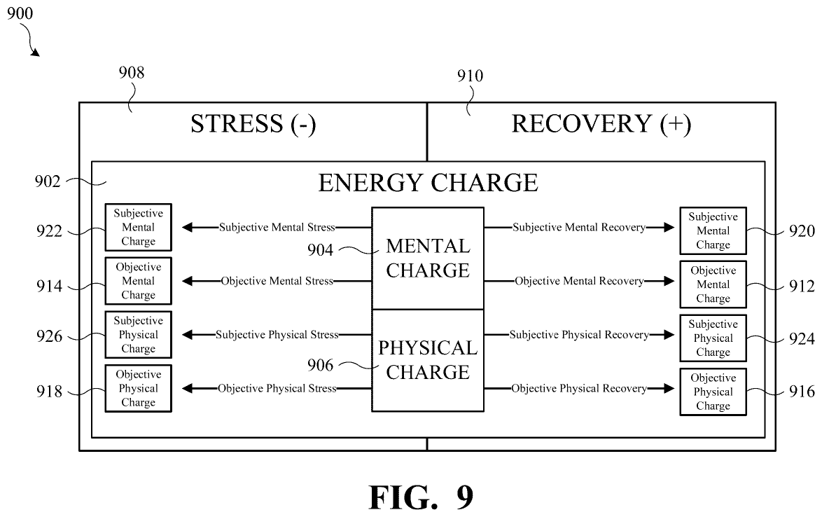

FIG. 9 illustrates a block diagram 900 depicting subjective and objective components that may be utilized to determine an energy charge 902 of a user. The energy charge 902 may be similar to the energy charge 402 and/or the energy charge 502 discussed above. As discussed above, the energy charge of the user may be determined and/or provided as an energy charge value that corresponds to an energy level of the user, which may be correlated to, associated with, or an indication of the user's physical and/or mental ability to partake in and/or complete a given activity. The energy charge 902 may be determined based on one or more components, such as a mental charge 904 and a physical charge 906. The mental charge 904 may be similar to the mental charge 404 and/or the mental charge 504 and the physical charge 906 may be similar to the physical charge 406 and/or the physical charge 506. Such components may be evaluated based upon periods of stress 908 and recovery 910, as further described herein.

As mentioned above, the energy charge 902 may be determined based upon subjective components and objective components. The objective components may correspond to determining the energy charge 902, the mental charge 904, and the physical charge 906 in accordance with the method 500 described above with respect to FIGS. 5-8.

For example, in a period of mental recovery (e.g., mediation), the mental charge 904 may be objectively evaluated using the method 500 (or another objective technique, such as another calculation) to determine the objective mental charge 912, which may correspond to an objective mental recovery of the user. Similarly, in a period of mental stress (e.g., a mentally stressful activity), the mental charge 904 may be objectively evaluated using the method 500 (or another objective technique, such as another calculation) to determine the objective mental charge 914, which may correspond to an objective mental stress on the user.

In a similar manner, in a period of physical recovery (e.g., sleep), the physical charge 906 may be objectively evaluated using the method 500 (or another objective technique, such as another calculation) to determine the objective physical charge 916, which may correspond to an objective physical recovery of the user. Similarly, in a period of physical stress (e.g., exercise), the physical charge 906 may be objectively evaluated using the method 500 (or another objective technique, such as another calculation) to determine the objective physical charge 918, which may correspond to an objective physical stress on the user.

In addition to the objective determinations of the mental charge 904 and the physical charge 906 as described above, the energy charge 902 may also, in certain cases, be partially or entirely based on subjective feedback provided by the user. For example, the user, via the device 100, the device 200, or another device, may provide feedback with respect to their subjective feeling of mental charge 904 and/or physical charge 906. The feedback may be in the form of answers to a questionnaire and/or other data manually input by the user. Such feedback may then be utilized as an additional or alternative metric to those determined based on the physiological signals detected by the device 100 or the device 200 (e.g., those determined based on the method 500). That is, the energy charge 902 of the user may be determined based on objective data (e.g., the physiological signals detected by the device 100 or the device 200), subjective data (e.g., the feedback from the user), or both. As a result, the energy charge 902 may be more tailored to the user and better reflect the feelings of the user.

For example, after a period of mental recovery (e.g., meditation), the user may manually input what they believe is the mental charge 904 as a subjective mental charge 920, which may correspond to a subjective mental recovery (e.g., a subjective feeling of mental recovery by the user). Similarly, after a period of mental stress (e.g., a mentally stressful activity), the user may manually input what they believe is the mental charge 904 as a subjective mental charge 922, which may correspond to a subjective mental stress (e.g., a subjective feeling of mental stress by the user).

Similarly, after a period of physical recovery (e.g., sleep), the user may manually input what they believe is the physical charge 906 as a subjective physical charge 924, which may correspond to a subjective physical recovery (e.g., a subjective feeling of physical recovery by the user). Similarly, after a period of physical stress (e.g., exercise), the user may manually input what they believe is the physical charge 906 as a subjective physical charge 926, which may correspond to a subjective physical stress (e.g., a subjective feeling of physical stress by the user).

The energy charge 902 of the user may be based on both the objective and subjective determinations of the mental charge 904 and/or the physical charge 906. By way of example, the objective determinations of the mental charge 904 and/or the physical charge 906 may be adjusted based upon the user's subjectively input determinations of the mental charge 904 and/or the physical charge 906. Based on such inputs by the user, the mental charge 904 determination (e.g., calculation or estimation) and/or the physical charge 906 determination (e.g., calculation or estimation) may be modified or otherwise adjusted to take into account the user's input.

For example, the physical charge 906 determined using objective data (e.g., based on the method 500) may indicate the user currently has a higher level of physical charge, such as after a period of recovery. However, the user may provide feedback that they are currently feeling physically tired and inputs a lower subjective value for the physical charge 906. As a result, the energy charge 902, which utilizes the physical charge 906 as a component, may be adjusted (e.g., decreased) based upon the user's feedback. Similarly, or additionally, the energy charge 902 determined may be adjusted to account for dissimilarities between the objectively determined value of the physical charge 906 of the user and the subjectively input value of the physical charge 906 of the user. It should be noted that a similar process may be utilized when determining the mental charge 904 of the user.

FIG. 10 illustrates a flow diagram of an example of a method 1000 for dynamically determining a mental charge of a user, such as the mental charge 504, the mental charge 504, and the mental charge 904 described above. The method 1000 may be implemented to determine whether mental recovery (e.g., recovery of the mental charge) of the user occurs. As discussed above, the energy charge of the user, including the mental charge, may be or may take into account the readiness (e.g., the readiness score) of the user. For example, with respect to the plan 700 shown in FIG. 7, the readiness 712 may correspond to the energy charge of the user at the morning check 706. As such, the readiness 712 may correspond to an initial energy charge of the user to begin a day. However, the readiness 712 of the user—and thus the energy charge of the user—may dynamically change throughout the course of a day. As such, the method 1000 may implement various recovery mechanisms and integrate various sleep metrics, as described below, to more accurately determine the energy charge of the user by adjusting an initial energy charge (e.g., the readiness score) of the user over the course of a desired time period (e.g., a particular day). Thus, the method 1000 may more accurately determine mental recovery of the user such that the mental recovery may significantly correlate to the sleep quality (e.g., a sleep score or sleep quality index) and/or one or more of the sleep metrics utilized to determine the sleep quality.

The method 1000 may determine mental recovery of the user according to the SAFTE model (e.g., the SAFTE model 516), which may account for different demands (e.g., pressures) placed on the user during periods of wakefulness and periods of sleep. As such, utilizing the SAFTE model, sleep (e.g., nighttime sleep) as well as pressure-free periods of wakefulness may contribute to the mental recovery of the user (e.g., recovery of the mental charge of the user). For example, the method 1000 may determine the mental recovery of the user based upon sleep metrics, including sleep duration, sleep stages, and start time for sleep (e.g., nighttime sleep). Moreover, the method 1000 may also diversify recovery mechanisms utilized to determine the mental recovery of the user to account for different interactions with sleep and pressure scenarios experienced by the user. As such, the method 1000 described herein may reconcile differences found between the initial energy charge of the user (e.g., the readiness 712 or readiness score) and a sleep score of the user (e.g., a sleep score for nighttime sleep). Thus, the method 1000 may implement repletion mechanisms to accurately determine the mental recovery of the user by determining one or more of mental recovery that happens during sleep under lower pressure, mental recover that happens during sleep under high pressure, and mental recovery that occurs during wakefulness under low pressure. As such, the method 1000 may also determine that mental depletion of the user occurs during wakefulness under high pressure.

As shown in FIG. 10, the method 1000 may begin in response to an event detection at 1002. The event detection may correspond to an exertion (e.g., the exertion 508) shown in FIG. 5, may correspond to another activity or task in which the user commences and/or completes, or may correspond to dynamically updating the mental charge throughout the day. That is, the event detection may detect any activity, whether or not mental exertion occurs. For example, the event detection may correspond to a period of rest or sleep in which little to no mental exertion occurs, and instead mental repletion may occur.

Once event detection occurs at 1002, a pressure or stress (e.g., a mental pressure or stress) of the user may be calculated at 1004. The pressure may be a pressure or stress level (e.g., low, medium, high) of the user based upon the present event detected. The pressure or stress may be based upon one or more physiological conditions of the user (e.g., heart rate, heart rate variability, blood pressure, body temperature, physical movement, electrodermal activity, etc.), whereby the one or more physiological conditions of the user may be based upon one or more physiological signals detected by the device 100, the device 200, or another portable device of the user. That is, the pressure or stress level of the user may be based on the one or more sensor or physiological signals. Such a determination (e.g., calculation) of the pressure or stress may be represented by a value for the pressure or stress of the user. Examples of pressure or stress of the user and determinations thereof can be found in U.S. application Ser. No. 18/783,750, filed on Jul. 25, 2024, the contents of which are incorporated herein in their entirety for all purposes.

The pressure or stress (e.g., the pressure or stress value) calculated at 1004 may also take into account physical exertion of the user. For example, the pressure calculated at 1004 may also integrate physical exertion of the user at 1006 to more accurately reflect the mental stress of the user, which may be directly impacted by the physical stress of the user. Integration of the physical exertion of the user at 1006 may be based upon any technique or calculation, and may be based upon one or more physiological conditions of the user (e.g., heart rate, heart rate variability, blood pressure, body temperature, physical movement, etc.), whereby the one or more physiological conditions of the user may be based upon one or more physiological signals detected by the device 100, the device 200, or another portable device of the user. For example, the physical exertion of the user at 1006 may be or may include an exertion metric, which may be calculated or determined based upon the one or more sensor or physiological conditions obtained by the device 100, the device 200, or another portable device of the user. The physical exertion of the user at 1006 may be determined based upon the method 500 described above, which may be responsive to an exertion 508 of the user. That is, the method 500 described above may be utilized to determine 1006. the effect of exertion on the user's energy level and/or the user's mental energy, and such a determination can subsequently be used in method 1000 at 1006. Therefore, the pressure load or pressure calculated at 1004 can be adjusted by the physical exertion of the user integrated at 1006, or that physical strain can be taken into account. For example, the pressure load or pressure calculated at 1004 can be adjusted (e.g., modified) based on the above exertion indicators to obtain the user's modified pressure load or pressure (e.g., the final calculated pressure load or pressure at 1004). Therefore, the user's mental energy and/or mental health can be based on stress load or stress level (e.g., stress load or stress level based on modification of exertion indicators).

After calculation of the pressure or stress at 1004 and integration of the physical exertion of the user at 1006 (e.g., modifying the pressure or stress level at 1004 based upon an exertion metric determined at 1006), sleep data may be obtained and/or calculated. For example, the sleep data may be based on one or more sensor or physiological signals obtained by the device 100, the device 200, or another device of the user. As such, the mental energy of the user may be based on the pressure or stress level as described above and the sleep data of the user.

Sleep data can be obtained and/or calculated after calculating the stress load or stress level at 1004 and integrating the user's fatigue at 1006 (e.g., modifying the stress load or stress level at 1004 based on the exertion indicators determined at 1006). For example, sleep data can be based on one or more sensor signals or physiological signals acquired by device 100, device 200, or other devices of the user. Thus, the user's mental energy can be based on the aforementioned stress load or stress level and the user's sleep data.

A sleep propensity of the user may be calculated at 1010. The sleep propensity (e.g., a sleep tendency) of the user may be calculated based on the circadian rhythm determined at 1008 as described above. In some other examples, the sleep propensity of the user may be calculated based on the user's personal data, historical sleep data, or the like. For example, the sleep propensity may be calculated as an inversion around the x-axis of the time cosine function with a predefined value of a parameter.

A sleep debt of the user may be calculated at 1012. The sleep debt of the user may be the difference between the amount of sleep the user needs and the amount of sleep the user gets, which may be determined cumulatively based on a cognitive energy reserve (e.g., a mental energy reserve or charge) of the user at the current time. For example, the according to the currently available mental charge of the user, the sleep debt of the user may be estimated or calculated according to a linear equation with a predefined slope.

A sleep intensity of the user may be calculated at 1014. The sleep intensity may be an indication of the intensity (e.g., quality) of the sleep of the user and may be determined based on at least one of the sleep propensity factors determined at 1010 as described above, the sleep debt determined at 1012 as described above, or the pressure or stress determined at 1004. For example, the sleep intensity of the user is determined based on a difference between the pressure or stress value of the user and a pressure or stress threshold (e.g., a predefined threshold value). For another example, the sleep intensity of the user is determined based on an accumulation of the sleep propensity and the sleep debt. In another example, the sleep intensity of the user is determined by summing the sleep propensity and the sleep debt, which are then multiplied by a natural exponent of the different between the current pressure or stress of the user (e.g., calculated at 1004) and predefined pressure or stress threshold multiplied by an expansion parameter.

The pressure or stress values may vary according to the expansion parameter with the highest expansion resulting in a lower pressure or stress natural exponent at high pressures or stresses. As such, when the expansion parameter is fixed at 1, the sleep intensity of the user varies according to the level of pressure or stress over the 48-hour period of time, and a constant decline in mental energy (e.g., sleep debt) may occur. Moreover, it should be noted that the sleep intensity may be capped at a maximum predefined value. The maximum predefined value may be any value, such as a value equivalent to about 50% of the initial sleep propensity when no pressure or stress is experienced by the user.

The pressure or stress determined (e.g., calculated) at 1004 is compared to a pressure or stress threshold (e.g., a predefined pressure threshold value) at 1016. If the pressure or stress determined (e.g., calculated) at 1004 is greater than or equal to the predefined pressure or stress threshold value, different charge mechanisms can be activated depending whether sleep may be detected or not at 1018. Conversely, if the pressure or stress determined (e.g., calculated) at 1004 is less than the predefined pressure or stress threshold value, mental charge mechanisms can be activated depending whether sleep may be detected or not at 1020.

Determining whether the user is sleeping may be based upon motion data, PPG data, heart rate data, one or more of the components calculated (e.g., circadian rhythm, sleep propensity, sleep debt, and sleep intensity), the pressure experienced by the user (e.g., calculated at 1004), other factors (e.g., one or more physiological conditions of the user, which may be based upon one or more physiological signals detected by the device 100 or another portable device of the user), or a combination thereof.

If it is determined at 1018 that the user is sleeping, the sleep of the user may be evaluated at 1022 and a determination of chronic daily stress, a measure of exercise load to estimate chronic fatigue, may be determined at 1024. Similarly, if it is determined at 1020 that the user is sleeping, the sleep of the user may be evaluated at 1026 and a determination of chronic daily stress may be determined at 1028. The evaluation at 1022 may be similar or the same as the evaluation at 1026. Similarly, the evaluation at 1024 may be similar or the same as the evaluation at 1028.

Evaluating the sleep at 1022 or 1026 may include determining the sleep state, the sleep duration, sleep regularity, sleep interruptions, sleep stages, sleep start time, or a combination thereof, as described above. Such determinations may be done based on one or more physiological conditions of the user, whereby the physiological conditions of the user may be based upon one or more physiological signals obtained by the device 100 when worn by the user or by another device.

Chronic daily stress determined at 1024 or 1028 may include determining the accumulation of the daily exercise load the user experiences over a number of days or weeks. Such a determination may include evaluating historical physiological conditions and/or signals to identify patterns over the course of days, weeks, months, or years. As such, chronic daily stress may be identified and taken into account when evaluating the over quality of sleep at 1022 and/or 1026. In principle, the level of chronic daily stress of the user may decrease the efficiency of mental charge recharging and may increase mental charge expenditure.

If sleep is not detected at 1018, chronic daily stress may be determined at 1030. Similarly, if sleep is not detected at 1020, chronic daily stress may be determined at 1032. Determinations of chronic daily stress at 1030 and/or 1032 may be done as described above with respect to 1024 and 1028.

Based on the sleep detection at 1018 or 1020—and the resultant sleep evaluation and/or chronic daily stress determination—the mental charge of the user may be determined at 1034. For example, if sleep is detected at 1018, then the mental charge determined at 1034 may be determined in accordance with a first sleep mental energy repletion mechanism, as indicated by the lead line 1036 between 1024 and 1034. The first mental energy repletion mechanism may be implemented to determine the recharge (e.g., increase) of the mental energy (e.g., the mental charge) of the user when the pressure calculated at 1004 exceeds the pressure threshold at 1016 and sleep is detected at 1018. For example, the first mental energy repletion mechanism may incorporate the sleep metrics determined at 1022 alongside an exponential decay expression to modulate the level of mental energy repletion as a function of the sleep of the user.

Conversely, if sleep is not detected at 1018, then the mental charge determined at 1034 may be determined in accordance with a wakefulness mental energy depletion mechanism, as indicated by the lead line 1038 between 1030 and 1034. The wakefulness mental energy depletion mechanism may be implemented to determine the depletion (e.g., decrease) of the mental energy (e.g., the mental charge) of the user when the pressure calculated at 1004 exceeds the pressure threshold at 1016 and sleep is not detected at 1018. As such, the wakefulness mental energy depletion mechanism may be implemented when the user awake and experiencing heightened levels of stress. The wakefulness energy depletion mechanism may include or may be a decay mechanism. For example, if the mental charge of the user is below about 50% the consumption rate or decay coefficient k of mental energy can be inversely proportional to the natural index of the user's current mental energy. For example, the consumption rate or decay coefficient of mental energy is determined by:

$$k = \frac{1.2}{a \times {e}^{{curr}_{battery}}} ,$$

where, A is a constant, curr_battery represents the user's current mental energy to increase mental energy loss because the user's mental energy is consumed more. In other examples, the rate of consumption or decay factor k of mental energy can be fixed if the user's mental energy is above 50%. In some embodiments, the expenditure of the user's mental energy can depend on the difference between the user's stress and the stress threshold. In some embodiments, the expenditure of the user's mental energy can be based on the difference between the user's stress and the pressure threshold and the product of the factor k.

Additionally, if sleep is detected at 1020, then the mental charge determined at 1034 may be determined in accordance with a second sleep mental energy repletion mechanism, as indicated by the lead line 1040 between 1032 and 1034. The second sleep mental energy repletion mechanism may be implemented to determine the recharge (e.g., increase) of the mental energy (e.g., the mental charge) of the user when the pressure calculated at 1004 does not exceed the pressure threshold at 1016 and sleep is detected at 1020. For example, the second sleep mental energy repletion mechanism may incorporate the sleep metrics determined at 1026 alongside an exponential decay expression to modulate the level of mental energy repletion as a function of sleep of the user.

Conversely, if sleep is not detected at 1020, then the mental charge determined at 1034 may be determined in accordance with a wakefulness mental energy repletion mechanism, as indicated by the lead line 1042 between 1028 and 1034. The wakefulness mental energy repletion mechanism may be implemented to determine the recharge (e.g., increase) of the mental energy (e.g., the mental charge) of the user when the pressure calculated at 1004 does not exceed the pressure threshold at 1016 and sleep is not detected at 1020. For example, the wakefulness mental energy repletion mechanism may remove an expansion parameter to decrease awake recharging of the mental energy compared to recharge which may occur during sleep. That is, the wakefulness mental energy repletion mechanism may adjust a rate of recharge of the mental energy compared to if the user is sleeping to more accurately account for the user still being awake.

Once the mental charge is determined at 1034, the mental charge may be further evaluated based upon one or more additional components. For example, an HRV score (e.g., value) may be determined at 1044, an RHR score (e.g., value) may be determined at 1046, an apnea-hypopnea index (AHI) score (e.g., value) may be determined at 1048, and a body temperature score (e.g., value) may be determined at 1050. As discussed above, such components may be determined based upon one or more physiological conditions of the user, whereby such physiological conditions may be based upon one or more physiological signals obtained by the device 100 when worn by the user or another device.

The mental charge determined at 1034 may thus be further adjusted and/or evaluated based upon the determinations at 1044-1040 to more accurately determine a final mental charge (i.e., a final mental charge value) at 1052. As such, factors that may conventionally impact and/or correlate to physical fatigue (e.g., based upon physical exertion) may be taken into consideration to evaluate their impact on mental fatigue of the user, which may correspond to or otherwise impact the mental charge of the user. As such, the final mental charge determined at 1052 may provide a more accurate calculation of the mental charge of the user compared to the mental charge determined at 1034.

FIG. 11 illustrates a flow diagram of an example of a method 1100 for dynamically determining a physical charge of a user, such as the physical charge 406, the physical charge 506, and the physical charge 906 described above. The method 1100 may be implemented to determine whether physical recovery (e.g., recovery of the physical charge) of the user occurs. As discussed above, the energy charge of the user, including the physical charge, may be or may take into account the readiness (e.g., the readiness score) of the user. For example, with respect to the plan 700 shown in FIG. 7, the readiness 712 may correspond to the energy charge of the user at the morning check 706. As such, the readiness 712 of the user—and thus the energy charge of the user—may dynamically change throughout the course of a day. As such, the method 1100 implement various recovery mechanisms and integrate various sleep metrics, as described below, to more accurately determine the energy charge of the user by adjust an initial energy charge (e.g., the readiness score) of the user over the course of a desired time period (e.g., a particular day). Thus, the method 1100 may accurately determine the physical recovery of the user such that the physical recovery may significantly correlate to the sleep quality (e.g., a sleep score) and/or one or more sleep metrics utilized to determine the sleep quality. Similarly, the method 1100 may take into account exercise intensity, such as by incorporating power of the user as an exertion indicator.

The method 1100 may also diversify depletion and/or recovery mechanisms utilized to determine the physical recovery of the user to account for different interactions with sleep and pressure scenarios experienced by the user. Thus, the method 1100 may implement repletion mechanisms to accurately determine the physical recovery of the user by determining: recovery that happens during sleep, above or below critical points, with higher recharging efficiency; recovery at rest with efficiency that is lower than recovery that may occur during sleep; or both.

As shown in FIG. 11, the method 1100 may begin in response to an event detection at 1102. The event detection may correspond to an exertion (e.g., the exertion 508) shown in FIG. 5, may correspond to another activity or task in which the user commences and/or completes, or may correspond to dynamically updating the physical charge throughout the day. That is, the event detection may detect any activity, whether or not physical exertion occurs. For example, the event detection may correspond to a period of rest or sleep in which little to no physical exertion occurs, and instead physical repletion may occur. As such, the method 1100 may determine the physical energy level (e.g., the physical charge) of the user after a particular event, such as exercise or napping.

As discussed above, the physical charge of the user may be determined based on periods of stress and periods of recovery of the user, whereby the user may alternate between the periods of stress and periods of recovery. The physical charge may also be based on, or take into account, an intensity (e.g., low, moderate, high) of the activity (e.g., the event detected at 1102) since different levels of intensity of a particular activity may consume different amounts of the user's physical charge, thereby depleting the overall physical charge of the user, which may thus impact the overall energy charge of the user.

Physical recovery (e.g., recovery of the physical charge) may be determined according to a heart rate reserve (HRR) and accelerometer data. By way of example, the physical charge of the user may be determined (e.g., calculated) based on the critical point model of heart rate data and/or exercise intensity and physical energy reserve. Physical energy consumption may occur when the heart rate and/or intensity of the activity is higher than a critical point (e.g., a predefined threshold), whereas physical recovery may occur when the physical energy consumption is lower than the critical point. That is, recharging of physical energy may occur when the HRR and accelerometer data do not exceed particular thresholds, as discussed further below. Such determinations may be made based on the exercise power of the user and/or data associated with the activity level of the user (e.g., data obtained by an accelerometer or other sensor of the device 100 or the device 200 when worn by the user).

The real-time exercise intensity or physiological exercise power of the user may be determined and based at least partially upon heart rate data, such as a heart rate reserve (HRR) of the user determined at 1106 and accelerometer data determined at 1108 (e.g., accelerometer data obtained by the device 100, the device 200, or another portable device of the user). The HRR of the user may be utilized to assess the physical fitness (e.g., the cardiovascular fitness) of the user to determine the user's real-time exercise intensity or physiological exercise power based upon a particular activity (e.g., based upon a particular exercise intensity). The real-time exercise intensity or physiological exercise power of the user may then be used to assess the activity level of the user and thereby determine the physical charge of the user.

It should also be noted that additional metrics may also be utilized to real-time exercise intensity or physiological exercise power of the user to thereby assess the activity level of the user and determine the physical charge of the user. By way of example, in lieu of, or in addition to, HRR, the present teachings may also contemplate determining the heart rate recovery and/or heart rate intensity of the user. Thus, the HRR of the user as described herein is only intended as an example metric for determining the physical charge.

As shown in FIG. 5, the real-time HRR determined at 1106 may be monitored at 1110 to determine if the HRR exists (i.e., is present) at that particular point in time (t). HRR may be or may be associated with the rate at which the user's heart rate returns to normal after a physical activity (e.g., after exercising). At 1110, the method 1100 determines whether the real-time HRR is present—that is, whether calculating the real-time HRR may be possible for the user based upon the current activity and/or status of the user.

If the real-time heart rate data exists at 1110, the real-time HRR may be determined (e.g., calculated or estimated) at 1112 for a time (t) based upon at least one of a resting heart rate of the user, a max heart rate of the user, and a current heart rate of the user (e.g., a real-time heart rate of the user). For example, the real-time HRR may be determined based on a difference between the current heart rate of the user and the max heart rate of the user, a difference between the current heart rate of the user and the resting heart rate of the user, a difference between the max heart rate and the resting heart rate of the user, or any combination thereof. It should be noted that the user's heart rate and/or the resting heart rate of the user may be determined (e.g., measured or estimated) based on one or more physiological signals detected by the device 100 or another portable device of the user. Additionally, the max heart rate of the user may be estimated based upon the user's personal data such as age (which may be manually input by the user via the device 100 or the device 200).

If the real-time heart rate data does not exist at 1110 (e.g., the real-time HRR is not able to be calculated based upon the current conditions), a default HRR value may be set at 1114. By way of example, the default HRR value set at 1114 may be 15. However, any default HRR value may be possible using the method 1100. Alternatively, the HRR value at the current time point may be deduced by using an algorithm based on the historical HRR data, the real-time HRR values at other time points close to the current time point, or the like, for example.

Once the real-time HRR value is calculated at 1112 or the default HRR value is set at 1114, the value is then further evaluated based upon integrating the physical exertion of the user at 1116 to more accurately reflect the physical stress of the user, which may directly impact the HRR of the user. Integration of the physical exertion of the user at 1116 may be based upon any technique or calculation, and may be based upon one or more physiological conditions of the user (e.g., heart rate, heart rate variability, blood pressure, body temperature, physical movement, etc.), whereby the one or more physiological conditions of the user may be based upon one or more physiological signals detected by the device 100, the device 200, or another portable device of the user. By way of example, the physical exertion of the user at 1006 may be determined based upon the method 500 described above, which may be responsive to an exertion 508 of the user. That is, the method 500 described above may be utilized to determine an impact on the energy charge of the user and/or the mental energy charge of the user, and such a determination may then be implemented into the method 1100 at 1116. Thus, the real-time HRR value calculated at 1112 or the default HRR value set at 1114 may be adjusted by or take into account the physical exertion of the user integrated at 1116.

Once physical exertion of the user is integrated into the method 1100 at 1116, the real-time HRR value calculated at 1112 or the default HRR value set at 1114 compared to the predefined critical point (CP) at 1118. The CP may be any predefined threshold value that is compared to the HRR value—either calculated at 1112 or set at 1114—to determine whether the HRR value is greater than or equal to the CP. The CP may be the same as or different from the default HRR value set at 1114. For example, the CP may also be 15. Thus, the CP may be considered a threshold HRR value. In some examples, the CP may be a customized value for the user and may be determined based at least partially on the user's personal data, history physiological data of the user, or the like.

If the HRR value is determined at 1118 to be greater than or equal to the CP, the activity level of the user (e.g., the event detected at 1102) may be evaluated. As discussed above, the real-time accelerometer measurements determined at 1108 may be used to evaluate the activity level (e.g., low, intermediate, high) of the user. For example, it may first be determined at 1120 whether such accelerometer measurements exist.

If the real-time accelerometer measurements exist, a real-time activity level may be calculated at 1122 based upon the real-time accelerometer measurements. For example, the real-time activity level may be calculated at 1122 based on at least one of a speed of movement of the user, an amount of movement of the user, a duration of time of movement of the user, one or more physiological conditions of the user (e.g., heart rate, blood pressure, breathing conditions, body temperature of the user, etc.), or one or more physiological signals detected by the device 100 or another portable device of the user. Such a determination at 1122 may be compared to one or more ranges of activity level (e.g., low, intermediate, high) to categorize the activity level accurately.

If the real-time accelerometer measurements do not exist at 1120, the activity level may be set at a value equal to a predefined threshold at 1124. For example, the predefined threshold may be a level of the activity that is sufficient for the user to exert physical energy such that physical recovery is not possible. By way of example, the predefined threshold may be categorized as a low activity level, whereby if the activity level is low, the user may be active yet not exerting excessive physical energy such that at least some physical recovery is possible (though not as much physical recovery as if the user was not partaking in any activity (e.g., resting)). In some examples, the activity level may be deduced based on historical motion data, historical activity level data of the user, or the like.

Once the activity level is calculated at 1122 or set equal to the threshold at 1124, the activity level is then compared to the threshold at 1126 to determine whether the activity level is greater than the threshold. Using the critical point model, multiple hypotheses may exist to determine whether the user is in a state of physical recovery or physical energy depletion based upon comparing the activity level to the threshold at 1126. Determining whether the user is in a state of physical recovery or physical energy depletion may also be based upon comparing the HRR value to the CP at 1118. Such a determination may then be used to determine (e.g., calculate or estimate) the physical charge of the user at the current time point (t) 1128. The physical charge of the user at the current time point (t) may also be compared to the physical charge of the user at a previous time point (t−1) 1104 to determine whether the user is in a state of physical recovery or physical energy depletion. It should be noted that the comparison of physical energy levels of the user between the current time point (t) 1128 and the previous time point (t−1) 1104 may be done before, during, or after determining a rate of physical recovery or physical energy depletion.

Illustrative scenarios will now be described with respect to the method 1100 shown in FIG. 11. In a first scenario, it may be determined based upon a first hypothesis of the critical point model that physical energy depletion occurs where the HRR value is greater than or equal to the CP. That is, physical energy depletion may occur when the HRR value is greater than or equal to the CP at 1118 and the activity level (e.g., exercise intensity) is greater than the threshold at 1126. In such a scenario, sleep may then be detected at 1130. Sleep may be detected at 1130 in a similar manner to the techniques described above with respect to sleep detection at 1018 or 1020 of the method 1000 shown in FIG. 10.

If sleep is detected at 1130 (i.e., the user is sleeping), the sleep of the user may be evaluated at 1132 and a determination of chronic daily stress may be determined at 1134. If sleep is not detected at 1132, a determination of chronic daily stress may be determined at 1136. Sleep may be evaluated at 1132 in a similar manner to the techniques described above with respect to sleep evaluation at 1022 or 1026 of the method 1000 of FIG. 10. Moreover, chronic daily stress may be determined at 1134 or 1136 in a similar manner to the techniques described above with respect to chronic daily stress determination at 1024, 1028, 1030, 1032 of the method 1000 of FIG. 10.

In the first scenario, the physical charge at time (t) at 1128 may be determined in accordance with physical energy depletion based upon at least one of the physical energy level of the user at the previous time point (t−1), the HRR at the current time point (t) (e.g., determined as described above), the HRR at the previous time point (t−1) (e.g., determined as described above), the CP, or an energy consumption rate of the user based on the heart rate conditions (e.g., max heart rate, resting heart rate, history heart rate data, etc.) of the user. It should be noted that the energy consumption rate may be correlated to the amount of time it takes the user to deplete or recover (i.e., recharge) his/her physical charge based upon a particular activity and/or current circumstances. The energy consumption rate may be determined based on a difference between the HRR and the CP, e.g., a bigger difference between the HRR and the CP corresponds to a greater energy consumption rate, or vice versa. Alternatively, the energy consumption rate is a preset value. In some examples, the physical charge at time (t) at 1128 may be determined based on a difference between the HRR at the current time point (t) or the HRR at the previous time point (t−1) with the CP. In some examples, the physical charge at time (t) at 1128 may be determined based on a sum of the physical energy level of the user at the previous time point (t−1) and the energy consumption rate of the user. In some examples, the physical charge at time (t) at 1128 may be determined based on the physical energy level of the user at the previous time point (t−1) as well as an energy consumption amount of the user, where the energy consumption amount may be determined based on the HRR at the current time point (t) or the HRR at the previous time point (t−1).

In the first scenario, if sleep is detected at 1130, the physical charge of the user determined at current time point (t) 1128 may be determined in accordance with a physical recovery during sleep mechanism, as indicated by the lead line 1158 between 1134 and 1128. The physical recovery during sleep mechanism may be implemented to determine the recharge (e.g., increase) of the physical energy (e.g., the physical charge) of the user when the HRR calculated at 1112 or set at 1114 is greater than or equal the CP at 1118, the activity level calculated at 1122 or set at 1124 is greater than the activity threshold at 1126, and sleep is detected at 1130. For example, the first physical recovery during sleep mechanisms may incorporate the sleep metrics determined at 1132 alongside an exponential decay expression to modulate the level of physical energy repletion as a function of the sleep of the user. By way of example, the physical recovery during sleep mechanism may be controlled by a set of exponential functions that may vary depending on the physical charge at the previous time point (t−1) 1104. For example, if the physical charge at the previous time point (t−1) 1104 is between 40% and 80%, the physical recovery during sleep mechanism may be based upon the level of the HRR and nothing more (e.g., the higher the HRR, the lower the recovery). Conversely, when the physical charge at the previous time point (t−1) 1104 is lower than 40% or higher than 80%, the physical recovery during sleep mechanism may be based upon both the level of the HRR and the accelerometer data obtained at 1108 (e.g., the higher the prior expenditure of the user, the more negative the recovery with higher HRR (e.g., more expenditure)).

Conversely, if sleep is not detected at 1130, then the physical charge of the user determined at current time point (t) 1128 may be determined in accordance with a physical energy depletion mechanism, as indicated by the lead line 1160 between 1136 and 1128. The physical energy depletion mechanism may be implemented to determine the depletion (e.g., decrease) of physical energy (e.g., the physical charge) of the user when the HRR calculated at 1112 or set at 1114 is greater than the CP at 1118, the activity level calculated at 1122 or set at 1124 is greater than the activity threshold at 1126, and sleep is not detected at 1130. For example, the physical energy depletion mechanism may be implemented when the user is awake and experiencing heightened levels of activity and stress. The physical energy depletion mechanism may include or may be a decay mechanism. For example, the exponential decay expression may follow a linear equation that multiplies the difference of the HRR percentage from its threshold to a decay rate. Thus, the larger the value of the decay rate, the larger the physical energy expenditure at higher HRR levels. Consequently, the value of the decay rate may be modulated according to the intensity of the activity that leads the HRR calculated at 1112 or set at 1114 to exceed their respective thresholds. Such modulation may be determined based upon exertion integrated at 1116 (e.g., using the method 500 described above).

In a second scenario, it may be determined based upon a second hypothesis of the critical point model that physical recovery occurs when the HRR value is less than the CP at 1118. In such a scenario, sleep may be detected at 1138. Sleep may be detected at 1138 in a similar manner to the techniques described above. If sleep is detected at 1138 (i.e., the user is sleeping), the sleep of the user may be evaluated at 1140 and a determination of chronic daily stress may be determined at 1142. If sleep is not detected at 1138, a determination of chronic daily stress may be determined at 1144. Sleep may be evaluated at 1140 and chronic daily stress may be determined at 1142 or 1144 in a similar manner to the techniques described above.

In the second scenario, the physical charge at time (t) at 1128 may be determined in accordance with a first physical recovery. The physical charge at time (t) may be determined based upon at least one of the physical energy level of the user at the previous time point (t−1), the HRR at the current time point (t) (e.g., determined as described above), the HRR at the previous time point (t−1) (e.g., determined as described above), or the CP. In some examples, the physical charge at time (t) at 1128 may be determined based on a difference between the HRR at the current time point (t) or the HRR at the previous time point (t−1) with the CP. In some examples, the physical charge at time (t) at 1128 may be determined based on a difference between the HRR at the current time point (t) or the HRR at the previous time point (t−1) with the CP. In some examples, the physical charge at time (t) at 1128 may be determined based on the physical charge of the user at the previous time point (t−1) and an energy recovery rate, where the energy recovery rate may be determined based on at least one of the HRR value, the physical charge of the user at the previous time point (t−1) at 1104, or the physiological conditions of the user. In some examples, the energy recovery rate is a preset value.

In the second scenario, if sleep is detected at 1138, the physical charge of the user determined at current time point (t) 1128 may be determined in accordance with the physical recovery during sleep mechanism, as indicated by the lead line 1162 between 1142 and 1128. The physical recovery during sleep mechanism may be implemented to determine the recharge (e.g., increase) of the physical energy (e.g., the physical charge) of the user when the HRR calculated at 1112 or set at 1114 is less than the CP at 1118 and sleep is detected at 1138. The physical recovery during sleep mechanism may be the same or similar to the physical recovery during sleep mechanism described above with respect to the first scenario (e.g., with respect to the lead line 1158). For example, the physical recovery during sleep mechanism may incorporate the sleep metrics determined at 1140 alongside an exponential decay expression to modulate the level of physical energy repletion as a function of the sleep of the user.

Conversely, if sleep is not detected at 1138, the physical charge of the user determined at current time point (t) 1128 may be determined in accordance with a physical recovery at rest mechanism, as indicated by the lead line 1164 between 1144 and 1128. The physical recovery at rest mechanism may be implemented to determine the recharge (e.g., increase) of the physical energy (e.g., the physical charge) of the user when the HRR calculated at 1112 or set at 1114 is less than the CP at 1118 and sleep is not detected at 1138. For example, the recovery at rest mechanism may remove an expansion parameter to decrease awake recharging of the physical energy compared to recharge which may occur during sleep. That is, the physical recovery at rest mechanism may adjust a rate of recharge of the physical energy compared to if the user is sleeping to more accurately account for the user still being awake. The physical recovery at rest mechanisms may also include an exponential decay mechanism similar to the ones described above with respect to the physical recovery during sleep mechanism.

In a third scenario, it may be determined based upon a third hypothesis of the critical point model that physical recovery occurs when the user's physical charge follows a time-exponential function relationship. That is, physical recovery may occur when the HRR value is greater than or equal to the CP at 1118 and the activity level is less than the threshold at 1126. In such a scenario, sleep may be detected at 1146. If sleep is detected at 1146 (i.e., the user is sleeping), the sleep of the user may be evaluated at 1148 and a determination of chronic daily stress may be determined at 1150. If sleep is not detected at 1146, a determination of chronic daily stress may be determined at 1152. Sleep may be evaluated at 1148 and chronic daily stress may be determined at 1150 or 1152 in a similar manner to the techniques described above.

In the third scenario, the physical charge at time (t) at 1128 may be determined in accordance with a second physical recovery mechanism. The physical charge at time (t) may be determined based upon at least one of the physical charge of the user at the previous time point (t−1) at 1104, the HRR at the current time point (t) (e.g., determined as described above), or the HRR at the previous time point (t−1). The physical energy recovery in this scenario is slower than the second scenario. That is, an energy recovery rate for this scenario is less than that in the second scenario. In some examples, the energy recovery rate may be determined based on at least one of the HRR value, the physical charge of the user at the previous time point (t−1) or the physiological conditions of the user. In some examples, the energy recovery rate is a preset value.

In the third scenario, if sleep is detected at 1146, the physical charge of the user determined at current time point (t) 1128 may be determined in accordance with the physical recovery during sleep mechanism, as indicated by the lead line 1166 between 1150 and 1128. The physical recovery during sleep mechanism may be implemented to determine the recharge (e.g., increase) of the physical energy (e.g., the physical charge) of the user when the HRR calculated at 1112 or set at 1114 is greater than or equal to the CP at 1118, the activity level calculated at 1122 or set at 1124 is less than or equal to the activity threshold at 1126, and sleep is detected at 1146. The physical recovery during sleep mechanism may be the same or similar to the physical recovery during sleep mechanism described above with respect to the first scenario and the second scenario (e.g., with respect to the lead line 1158 and the lead line 1162). For example, the physical recovery during sleep mechanism may incorporate the sleep metrics determined at 1150 alongside an exponential decay expression to modulate the level of physical energy repletion as a function of the sleep of the user.

Conversely, if sleep is not detected at 1146, the physical charge of the user determined at current time point (t) 1128 may be determined in accordance with a physical recovery at rest mechanism, as indicated by the lead line 1168 between 1152 and 1128. The physical recovery at rest mechanism may be implemented to determine the recharge (e.g., increase) of the physical energy (e.g., the physical charge) of the user when the HRR calculated at 1112 or set at 1114 is greater than or equal to the CP at 1118, the activity level calculated at 1122 or set at 1124 is less than or equal to the activity threshold at 1126, and sleep is not detected at 1146. The physical recovery at rest mechanism may be the same or similar to the physical recovery at rest mechanism described above with respect to the second scenario (e.g., with respect to the lead line 1164).

Based upon the physical energy consumption determined above in accordance with one of the three scenarios (e.g., based upon one or more of the aforementioned hypotheses), the physical charge of the user at the current time point (t) may be determined at 1128 based upon the physical energy consumption or recovery of the user. Thus, based on the method 1100 described above, the physical charge of the user may be determined (e.g., calculated) to assess whether the user is in a state of recovery or rest, and thereby provide one of the components utilized to determine the overall energy charge of the user.

Once the physical charge at time (t) at 1128 is determined, the physical charge may be further evaluated based upon one or more additional components, such as the additional components evaluated at 1154. For example, an HRV score (e.g., value), a RHR score (e.g., value), an apnea-hypopnea index (AHI) score (e.g., value), and a body temperature score (e.g., value) may be determined at 1154. As discussed above, such components may be determined based upon one or more physiological conditions of the user, whereby such physiological conditions may be based upon one or more physiological signals obtained by the device 100 when worn by the user or another device.

The physical charge at time (t) determined at 1128 may thus be further adjusted and/or evaluated based upon the determinations at 1154 to more accurately determine a final physical charge (i.e., a final physical charge value) at 1156. As such, the final physical charge determined at 1156 may provide a more accurate calculation of the physical charge of the user compared to the physical charge determined at 1128.

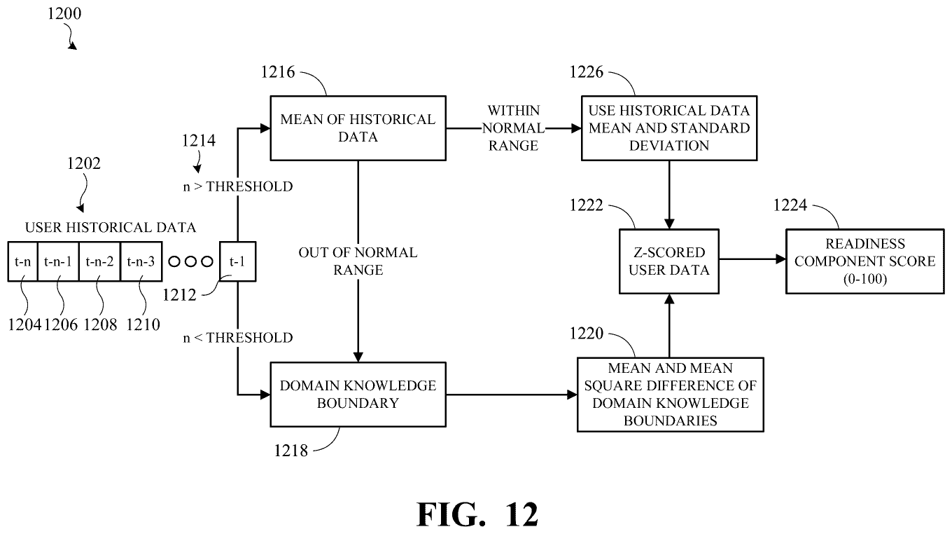

FIG. 12 illustrates a flow diagram of an example of a method 1200 for dynamically determining a readiness component score for one or more of the components described above. The readiness component score may be the readiness 712 of FIG. 7 discussed above. The method 1200 may also be equally applicable for dynamically determining and/or creating the conclusion 714 of FIG. 7 discussed above. That is, the readiness 712 may be at least partially based upon data from a previous day whereas the conclusion 714 may be at least partially based upon a data from a current day. In each case, historical user data may be utilized to determine the readiness (e.g., historical user data from a previous data) and the conclusion (e.g., historical user data from the current day). As such, the readiness (e.g., the readiness score) and the conclusion, or any components therein, may be assessed according to user historical data to calculate individualized statistical measures of the readiness and/or the conclusion for the various components. Moreover, the method 1200 may also account for insufficient historical data and/or deviation from a normal value range, as described in further detail below.

To further illustrate how any component utilized to determine the readiness or the conclusion of the user may be assessed to determine the readiness (e.g., the readiness score) or the conclusion, FIG. 12 will now be described in further detail. It should be noted that the generic term “component” will be used with respect to FIG. 12, which may represent any one or more of the components described above with respect to FIGS. 4-11, unless otherwise stated.

As described above, user historical data 1202 may be used to assess the component to determine the readiness score of the component or a value of the component for the conclusion. For example, as shown in FIG. 12, the user historical data 1202 may include one or more historical data points (e.g., data values) for different time points (e.g., represented as t-n, t-n-1, t-n-2, t-n-3, where t represents the current time point (t) and (n) represents the total number of data points (e.g., a total number of days, whereby each data point is taken on a different day)). The historical data points are illustrated as 1204, 1206, 1208, 1210, and 1212 in FIG. 12. It should be noted that the historical data points may represent one or more desired timescales. For example, the historical data points may be taken to represent a daily, weekly, monthly, or annual timescale of data points.

The assessment of the component may be done according to the user historical data 1202 to calculate the readiness score of the component or the value of the component for the conclusion if there is sufficient historical data and/or the historical data points are determined to fall within a normal range (e.g., a predefined normal range). To determine whether there is sufficient historical data, the user historical data 1202 may evaluated to determine whether the total number (n) of data points (e.g., the total number of days, whereby each day may include a data point) is greater than or less than a predefined threshold. The predefined threshold may be any desired value. For example, in the case where a data point of the condition is determined on a daily basis (e.g., n is equal to the number of days of evaluation of the user), the predefined threshold may be a month (e.g., the predefined threshold is equal to 30 days).

If it is determined that the total number (n) of data points is greater than the predefined threshold at 1214, the method 1200 may then calculate a mean of the user historical data 1202 at 1216. However, if it is determined that the total number (n) of data points is less than the predefined threshold at 1214, the method 1200 may then determine domain knowledge boundaries at 1218. The domain knowledge boundaries at 1218 may be predefined boundaries utilized to determine the readiness score of the component or the value of the component for the conclusion. That is, the domain knowledge boundaries may be limits or edges of possible values of the user data that may be used in lieu of the user historical data 1202 (e.g., where there is insufficient user historical data). Similarly, if the mean of historical data determined at 1216 is out of the normal range (e.g., the normal predefined range), domain knowledge boundaries may be determined at 1218. Thus, the method 1200 may take into account both insufficient user historical data and significant deviation in the user historical data.

Once the domain knowledge boundary is determined at 1218, a mean and standard deviation of the domain knowledge boundary may be determined at 1220. Based upon the mean and standard deviation of the domain knowledge boundary determined at 1220, a Z-score of the user data may be determined (e.g., calculated) at 1222. The Z-score of the user data may correspond to a statistical measurement that described how many standard deviations the user data (e.g., a data point of the user data) is from the mean, thereby facilitating a standardized readiness component score or value of the component for the conclusion for any of the components. That is, based on the Z-score determined at 1222, a readiness component score or component value in the range of 0 to 100 may be determined at 1224.

As described above, the readiness component score or the component value determined at 1224 may be based on the domain knowledge boundary determined at 1218 and the mean and standard deviation of the domain knowledge boundary determined at 1220 when the user historical data 1202 is insufficient or deviates too significantly. However, when the user historical data 1202 is determined to be sufficient (e.g., the total number (n) of data points is greater than the predefined threshold) and the mean of the historical data determined at 1216 is within the normal range, the readiness component score or the component value determined at 1224 may be based on the user historical data 1202.

For example, when the mean of the user historical data 1202 determined at 1216 is within the normal range, the mean of the user historical data and a standard deviation of the user historical data 1202 determined at 1226 may be used to determine the readiness component score or the component value. That is, the Z-score determined (e.g., calculated) at 1222 may be based upon the mean and the standard deviation of the user historical data 1202 instead of the mean and the standard deviation of the domain knowledge boundary. The Z-score determined at 1222 based upon the user historical data 1202 may then be used to determine the readiness component score or component value of the component at 1224.

Thus, using the method 1200 to determine the readiness component score or component value of each of the components, the overall readiness score or conclusion associated with the user may be based upon standardized component scores or values of the components. In particular, the component score or value may be a weighted average of the component scores or values determined for one or more of the components in accordance with the method 1200 described above. That is, the readiness score and the conclusion may be determined based on the scores or values associated with any one of the components described above. In some examples, one or more weight values for the components may be determined in accordance with their associated component scores, e.g., a smaller weight value is determined for a greater component score. In some examples, one or more weight values for the components are determined based on the user's personal data, user's historical data, or the like.

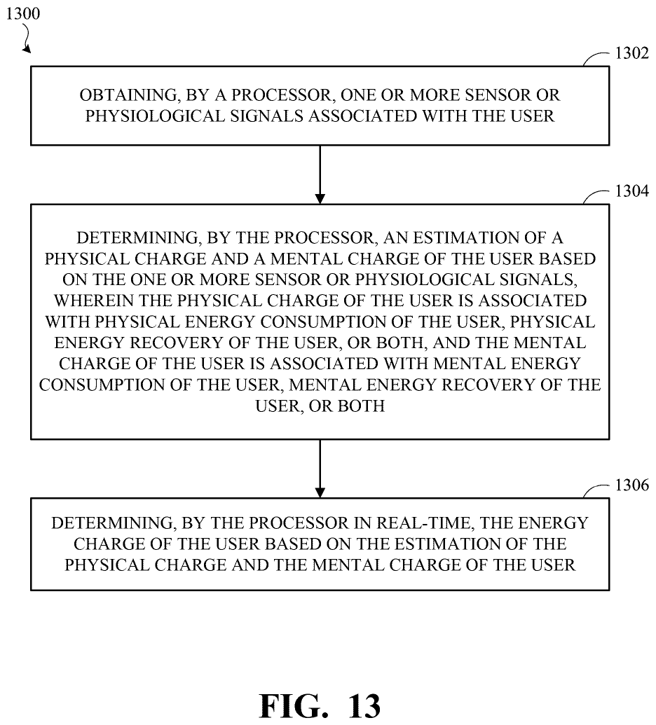

FIG. 13 is a flow diagram of a method 1300 for dynamically determining an energy charge of a user wearing a wearable device. For example, the method 1300 shown in FIG. 13 may be implemented using the device 100, the device 200, and/or one or more external devices in communication with the device 100 or the device 200. The method 1300 may be similar to, be part of, or include the method 500, the method 1000, the method 1100, the method 1200, or a combination thereof.

The method 1300 can be implemented as software and/or hardware modules in the computing device 300 of FIG. 3. For example, the method 1300 can be implemented as software modules stored in the storage 330 as instructions and/or data executable by the processor 305 of an apparatus, such as the device 100 in FIG. 1 or the device 200 in FIGS. 2A and 2B. In another example, the method 1300 can be implemented in hard as a specialized chip storing instructions executable by the specialized chip. Some or all of the operations of the method 1300 can be implemented by the processor 305 of FIG. 3.

The method 1300 includes obtaining, by a processor, one or more sensor or physiological signals associated with the user at 1302. The one or more sensor or physiological signals may be associated with one or more physiological conditions of the user described above. Additionally, the one or more sensor or physiological signals may be associated with one or more of the components shown in FIGS. 4-11 and described above.

The method 1300 also includes at 1304 determining, by the processor, an estimation of a physical charge and a mental charge of the user based on the one or more sensor or physiological signals. The physical charge of the user is associated with physical energy consumption of the user, physical energy recovery of the user, or both. Similarly, the mental charge of the user is associated with mental energy consumption of the user, mental energy recovery of the user, or both. The physical charge and the mental charge may be one or more of the components shown in FIGS. 4-11 that may be associated with the energy charge of the user. That is, the physical charge and the mental charge may be utilized to calculate an overall energy charge of the user.

The method 1300 may further include at 1306 determining, by the processor, the energy charge of the user based on the estimation of the physical charge and the mental charge of the user. For example, the energy charge of the user may be determined as described above with respect to FIGS. 6-13 (e.g., determined as a weighted average of the readiness component scores).

In some implementations, a measurement of energy charge of a user includes: acquiring one or more sensor or physiological signals associated with a user; determining or updating the user's energy charge based on the one or more sensor or physiological signals; and generating output information based on the user's energy charge.

In some implementations, the one or more sensor or physiological signals includes at least one of movement signals or physiological signals.

In some implementations, in response to detecting a preset time or a predefined event, determining or updating the user's energy charge based on the one or more sensor or physiological signals.

In some implementations, determining that a predefined event is detected based on the one or more sensor or physiological signals associated with the user; and in response to determining that the predefined event is detected (e.g., in response to occurrence of the predefined event), determining or updating the user's energy charge.

In some implementations, determining a charge change of the user according to at least one event parameter of the predefined event; and determining or updating the user's energy charge based on the user's charge change.

In some implementations, at least one event parameter of the predefined event is determined based on the one or more sensor or physiological signals associated with the user.

In some implementations, the predefined event includes at least one of exercise, sleep supplementation, acute stress, or significant stress.

In some implementations, determining the user's mental charge and physical charge based on the one or more sensor or physiological signals; and determining the user's energy charge based on the user's mental charge and physical charge.

In some implementations, determining a health assessment of the user based on the one or more sensor or physiological signals; and adjusting at least one of the user's mental charge or physical charge based on the health assessment to obtain the user's energy charge.

In some implementations, determining a change rate or amount of the user based on the one or more sensor or physiological signals associated with the user; and updating the user's energy charge based on the user's charge change.

In some implementations, determining an charge change state of the user based on the one or more sensor or physiological signals, wherein the charge change state includes at least one of a physical energy consumption state, a physical energy recovery state, a mental energy consumption state, or a mental energy recovery state; and determining or updating the user's energy charge based on the charge change state of the user.

In some implementations, determining at least one of the user's sleep data or an exertion metric according to the one or more sensor or physiological signals associated with the user, wherein the exertion metric is associated with the user's daily activities and/or exercise; and determining or updating at least one of the user's energy charge, physical energy, or mental energy according to at least one of the user's sleep data or the exertion metric.

In some implementations, the exertion metric includes an exercise power deduced based on at least one of HRR data during the exercise or the attribute information of the user (e.g., gender, age, BMI, or the like)

In some implementations, the sleep data includes at least one of sleep quality or sleep sequence.

In some implementations, determining an exertion metric of the user according to the one or more sensor or physiological signals associated with the user over a time period; tracking the exertion metric of the user over a plurality of the time periods to obtain the user's chronic physical stress; and obtaining at least one of the user's energy charge, physical charge, or mental charge according to the user's chronic physical stress.

In some implementations, a change rate or a charge change amount of the user is obtained at least in part according to the user's chronic physical stress, and the change rate includes at least one of a change rate of physical charge or a change rate of mental charge.

In some implementations, determining an exertion metric of the user according to the one or more sensor or physiological signals associated with the user; and determining or updating the user's mental charge according to the user's exertion metric.

In some implementations, determining an estimation of mental stress of the user according to the one or more sensor or physiological signals associated with the user; adjusting the estimation of the mental stress according to the user's exertion metric to obtain adjusted estimation of the mental stress of the user; and determining or updating the user's mental energy according to the adjusted estimation of the mental stress of the user.

In some implementations, determining an estimation of the mental stress of the user according to the one or more sensor or physiological signals associated with the user; and determining or updating the user's mental energy according to a difference between the estimation of the mental stress and a stress threshold.

In some implementations, the user's mental energy is obtained based on a target mental energy assessment model, and the target mental energy assessment model is selected from multiple preset mental energy assessment models based on at least one of a comparison result between the estimation of a mental stress of the user and the stress threshold or sleep data (e.g., sleep state indicator indicating whether the user is in a sleep state) of the user.

In some implementations, based on at least one of a comparison result between the estimation of the mental stress of the user and the stress threshold or sleep data (e.g., sleep state indicator indicating whether the user is in a sleep state) of the user, determining that the user is in a mental energy recovery state or a mental energy consumption state.

In some implementations, selecting a target mental energy assessment model from at least one mental energy supplementation model and at least one mental energy consumption model based on at least one of a comparison result between the estimation of the mental stress of the user and the stress threshold or sleep data (e.g., sleep state indicator indicating whether the user is in a sleep state) of the user.

In some implementations, determining that the user is in a mental energy recovery state based on a comparison result between the estimation of the mental stress of the user and the stress threshold, and determining a target mental energy assessment model from multiple mental energy supplementation models according to sleep data (e.g., a sleep state indicator indicating whether the user is in a sleep state, etc.) of the user.

In some implementations, in response to the estimation of the mental stress of the user exceeding the stress threshold and the user not being in a sleep state, determining that the user is in a mental energy consumption state.

In some implementations, in response to the estimation of the mental stress of the user not exceeding the stress threshold or the user being in a sleep state, determining that the user is in a mental energy recovery state.

In some implementations, determining or updating the user's physical charge based on the user's heart rate reserve and movement data.

In some implementations, determining or updating the user's physical charge based on the movement data, heart rate reserve, and sleep data (e.g., a sleep state indicator, etc.) of the user.

In some implementations, determining that the user is in a physical energy consumption state or a physical energy recovery state according to the sleep data (e.g., a sleep state indicator, etc.).

In some implementations, determining a target physical energy assessment model from multiple preset physical energy assessment models according to the sleep data (e.g., a sleep state indicator, etc.).

In some implementations, in response to at least one of the following being satisfied: the user's movement data indicating that the activity level of the user does not exceed an activity threshold, the user's heart rate data indicating that the heart rate reserve does not exceed a heart rate threshold, or the sleep data indicating that the user being in a sleep state, determining that the user is in a physical energy recovery state.

In some implementations, in response to the user's movement data indicating that the activity level of the user exceeds the activity threshold, the user's heart rate data indicating that the heart rate reserve exceeds the heart rate threshold, and the sleep data indicating the user not being in a sleep state, determining that the user is in a physical energy consumption state.

In some implementations, determining or updating the user's physical energy according to the user's exertion metric.

In some implementations, determining a change rate of the user's physical energy according to the user's exertion metric.

In some implementations, adjusting the user's heart rate reserve according to the user's exertion metric to obtain an adjusted heart rate reserve, and determining or updating the user's physical energy according to the adjusted heart rate reserve.

In some implementations, determining a change rate or change amount of the user's physical energy, adjusting the change rate or change amount of the user's physical energy according to the user's sleep data to obtain an adjusted change rate or change amount of the physical energy.

In some implementations, determining a target physical energy assessment model from multiple physical energy supplementation models according to sleep data (e.g., sleep state indicator indicating whether the user is in a sleep state) of the user, wherein the multiple physical energy supplementation models correspond to different change rates or change amounts.

In some implementations, the health assessment is obtained based on at least one of cardiac health, respiratory health, or body temperature.

In some implementations, the health assessment is obtained based on at least one cardiac health index, at least one respiratory health index, or body temperature of the user and respective weights.

In some implementations, the at least one cardiac health index includes at least one of resting heart rate, heart rate variability, or cardiac arrhythmia.

In some implementations, the at least one respiratory health index includes an apnea-hypopnea index.

In some implementations, determining respective weights corresponding to the user's mental energy and physical energy; and determining the user's energy charge according to the user's mental energy, physical energy, and their respective weights.

In some implementations, a measurement of energy charge of a user includes: acquiring one or more sensor or physiological signals associated with a user; detecting a predefined event based on the one or more sensor or physiological signals associated with the user; and determining or updating at least one of the physical energy, mental energy, or energy charge of the user based on the detected predefined event.

In some implementations, in response to detecting the predefined event, determining or updating at least one of the physical energy, mental energy, or energy charge of the user based on the detected predefined event.

In some implementations, the predefined event includes at least one of an exercise event, a sleep supplementation event, an acute stress event, or a significant stress event.

In some implementations, determining a charge change of the user according to at least one event parameter of the predefined event; and determining or updating at least one of the physical energy, mental energy, or energy charge of the user based on the user's charge change.

In some implementations, in a case that the detected preset time is sleep, determining that at least one of the physical energy, mental energy, or energy charge of the user is in a recovery state.

In some implementations, in a case that the detected predefined event is exercise, determining that at least one of the physical energy, mental energy, or energy charge of the user is in a consumption state or a recovery state based on movement data and heart rate data obtained from the one or more sensor or physiological signals.

In some implementations, in a case that the detected predefined event is acute or significant stress, determining that at least one of the physical energy, mental energy, or energy charge of the user is in a consumption state.

In some implementations, determining or updating at least one of the physical energy, mental energy, or energy charge of the user based on an estimated value of at least one sleep parameter of the user and at least one parameter of the predefined event.

In some implementations, determining that at least one of the physical energy, mental energy, or energy charge of the user is in an energy recovery state based on the predefined event; determining a target energy recovery model from multiple preset energy recovery models corresponding to the energy recovery state based on the estimated value of at least one sleep parameter of the user; and determining or updating at least one of the physical energy, mental energy, or energy charge of the user based on the target energy recovery model.

In some implementations, determining that at least one of the physical energy, mental energy, or energy charge of the user is in an energy recovery state based on the predefined event; determining an energy recovery rate of the user based on the estimated value of at least one sleep parameter of the user; and determining or updating at least one of the physical energy, mental energy, or energy charge of the user based on the energy recovery rate.

In some implementations, a measurement of energy charge of a user includes: acquiring one or more sensor or physiological signals associated with a user, wherein the one or more sensor or physiological signals includes at least one of movement data and physiological data; determining an estimated value of at least one sleep parameter of the user based on the one or more sensor or physiological signals; and determining or updating at least one of the physical energy, mental energy, or energy charge of the user based on the estimated value of the at least one sleep parameter of the user.

In some implementations, the at least one sleep parameter includes at least one of sleep state, sleep quality, sleep duration, sleep stages, or deep sleep duration or ratio.

In some implementations, determining that at least one of the physical energy, mental energy, or energy charge of the user is in a consumption state or a recovery state at least in part based on the estimated value of the at least one sleep parameter.

In some implementations, determining a change rate or change amount of at least one of the physical energy, mental energy, or energy charge of the user based on the estimated value of the at least one sleep parameter of the user.

In some implementations, a measurement of energy charge of a user includes: acquiring one or more sensor or physiological signals associated with a user, wherein the one or more sensor or physiological signals includes at least one of movement data or physiological data; determining an estimated value of the user's chronic physical stress based on the one or more sensor or physiological signals; and determining or updating at least one of the physical energy, mental energy, or energy charge of the user based on the estimated value of the user's chronic physical stress.

In some implementations, the system for dynamically determining the energy charge of the user may obtain an estimation of the energy charge of the user based on one or more sensor or physiological signals associated with the user obtained from at least one sensor of a wearable device. For example, the processor of the wearable device may receive the one or more sensor or physiological signals sent from at least one sensor. For another example, a processor of another electronic device (e.g., the intermediate device or the server device) may receive the one or more sensor or physiological signals sent by the wearable device. The one or more sensor or physiological signals may be raw signals detected by the at least one sensor of the wearable device, or be processed signals by performing one or more processing on the raw signals. The one or more sensor or physiological signals may include at least one of movement signals or physiological signals. Optionally, the physiological signals may include at least one of cardiac, respiratory, and body temperature signals. In some examples, the system may determine the user's energy charge based on the user's movement data (e.g., exercise intensity, exercise amount, exercise heart rate, exercise type, exercise time duration, exercise power, etc.), body temperature data, respiratory data, heart rate data (e.g., resting heart rate reserve (RHR), exercise heart rate, heart rate variability (HRV), heart rate reserve, etc.), sleep data (e.g., sleep staging, sleep sequence, sleep duration, deep sleep ratio or duration, sleep regularity, sleep quality, etc.), or the like, or any combination thereof, but the implementations of the present disclosure are not limited thereto.

In some implementations, the system determines an estimation of the user's energy charge at a predefined time point, such as regularly updating the user's energy charge in the morning, noon, and evening of a day.

In some implementations, the system may perform event-based estimation of the energy charge of the user to dynamically update the user's energy charge to adapt to specific events, such as exercise, naps, or acute or significant physiological pressure. In some examples, the system may update the user's energy charge based on detected exercise events. The system may adjust at least one of the user's physical, mental, or overall energy charge according to the user's exertion metric and/or energy consumption during exercise. For instance, during activities like running, the system may update the user's physical and/or overall energy charge based on exercise intensity and/or heart rate changes. Additionally, in some implementations, considering the link between physical and mental fatigue, the user's mental charge may also be updated based on the exercise intensity and/or heart rate changes. This update can record the user's energy consumption from the start of exercise or the last updating point to the end of exercise, thereby providing personalized insights for exercise events to help the user optimize recovery and exercise performance based on their own metrics and exercise intensity.

In another example, the system may update the user's energy charge based on detected nap events. The system may replenish at least one of mental, physical, or overall energy charge according to one or more parameters associated with the nap, such as nap time (e.g., start time, end time, time duration, etc.), nap quality, and so on. In some implementations, respective weights may be determined based on one or more nap parameters (e.g., by determining whether one or more nap parameters are abnormal), and at least one of the user's physical, mental, or overall energy charge may be updated based on the respective weights. Optionally, the system may also provide guidance to the user to help maintain energy or optimize remaining daily energy.

In another example, the system may update the user's energy charge based on detected physiological pressure events (e.g., acute or significant stress events). For instance, in response to detecting acute or significant stress, the system may adjust the user's mental and/or overall energy charge according to at least one stress parameter such as the intensity and duration of the stress event. Optionally, if a change in the user's heart rate is observed, the user's physical charge may be updated. Optionally, guidance may be provided to the user immediately during the stress event and/or a follow-up summary, and the guidance may include insights and suggestions may be provided after the stress event ends.

In some implementations, the system may also provide recommendations for the user based on the user's energy charge to guide daily or specific activities and optimize daily performance and recovery. For example, the system may generate and/or adjust the user's health plan, recovery plan, and/or training plan based on at least one of the user's physical, mental, or overall energy charge.

The above examples track and dynamically update the user's energy charge taking one day as an example. In some implementations, the assessment of the energy charge of the user may also be performed in other time units (e.g., weeks or months), which is not limited herein.

In some implementations, a chronic physical stress or load of the user during a second time period may be obtained by tracking the user's physical stress or load during the second time period comprising at least one first time period, and based on the chronic physical stress or load during the second time period, determining or updating at least one of the user's physical, mental or overall energy charge of the user. For example, a daily physical stress or load accumulation may be performed based on the cumulative of the user's daily training load TRIMP, and a daily physical stress or load accumulation of seven days may be averaged to generate a chronic daily physical stress.

In some implementations, the user's chronic physical stress or load is used to modify the energy replenishment (or recovery) mechanism, for example, the higher the chronic physical stress or load, the longer the user will need to recover in order to achieve full energy replenishment.

In some implementations, changes in at least one of the user's physical or mental charge may be determined based on a health assessment of the user. Optionally, the health assessment may be based on heart health, breath health, or body temperature. For example, the body temperature may include at least one of core temperature, skin temperature, average temperature, or the like.

### Determination of Mental Charge

The sleep quality of user's regular sleep (e.g., sleep at night) may affect energy replenishment. The physiological pressure of the user at day and night may affect energy expenditure. For Mental Charge, tracking of physical stress caused by an exertion metric may be performed, and HR-based activity data may be used in the physical stress calculation to improve completeness and accuracy. In some implementations, physical fatigue may affect mental fatigue. In some implementations, at least one of cardiac (e.g., RHR, HRV, etc.), respiratory (e.g., AHI) or temperature health may also be considered. More accurate mental charge calculations are generated based on sleep recovery, physical stress metrics, and different events and checkpoints throughout the day.

In some implementations, the user's mental charge may be obtained based on the SAFTE algorithm for reacting to different stressors faced by the user.

In some implementations, the user's mental charge value or mental charge consumption may be determined based on the user's exertion metric, e.g., per minute, for each of the at least one time period.

The user's mental charge may be updated periodically or based on detected events, for example, the user's mental charge consumption or mental charge value may be updated based on the user's exertion metric.

In some implementations, different physiological pressures exerted by the user on the individual during wakefulness and sleep may be considered. In some implementations, sleep (e.g., nap) as well as stress-free or low-stress periods of wakefulness may contribute to mental replenishment (or recovery).

In some implementations, a mental stress estimation of the user may be determined, e.g., based on the HRV data of the user, and based on the user's mental stress estimation, a mental charge expenditure or mental charge value of the user may be determined.

In some implementations, the mental stress estimation of the user may be obtained based on at least one of at least one sleep parameter (e.g., at least one of a sleep intensity, a circadian rhythm, a sleep propensity, or a sleep debt, etc.), HRV, RMSSD, heart rate, or movement data of the user. The user's mental stress estimation may be adjusted based on the user's exertion metric. For example, the user's mental stress estimation may be adjusted based on the overall exertion metric:

$${Pressure}_{final} = {Pressure}_{t} * {e}^{k * {Exertion}_{t}}$$

where Pressure_{final} is the adjusted mental stress estimation, Pressureₜ is the mental stress estimation of the user at t, Exertionₜ is the exertion metric of the user, and k is a preset value.

In some implementations, the mental charge of the user is updated or determined based on the user's mental stress estimation and sleep data (e.g., a sleep state indicator indicating whether the user is in a sleep state).

In some implementations, if sleep is detected, the mental charge of the user is updated or determined based on the at least one sleep parameter.

In some implementations, the change rate or amount of the user's mental charge is based on a difference between the user's mental stress estimation and a threshold value.

In some implementations, the physiological pressure assessment may be performed immediately after calculating the circadian rhythm to enhance the mental charge replenishment mechanism.

In some implementations, mental charge depletion occurs only during wakefulness under high stress.

In some implementations, mental charge replenishment (or recovery) occurs during both the user's sleep period as well as during wakefulness under low stress.

In some implementations, multiple mental charge replenishment mechanisms may be provided, such as a wakefulness-based mental charge replenishment mechanism, a sleep-based mental charge replenishment mechanism.

In some implementations, a target mental charge replenishment mechanism may be selected from the multiple mental charge replenishment mechanisms based on the user's sleep data (e.g., sleep state indicator).

In some implementations, if the user's mental stress estimation does not exceed a stress threshold and sleep is detected, a first sleep-based mental charge supplementation mechanism is selected. The mental energy replenishment at the current moment may be determined based on the mental energy replenishment at at least one previous moment and a recovery rate. In some examples, the recovery rate is determined based on at least one sleep parameter (e.g., a sleep Intensity, sleep duration, sleep start time, WASO(Wake After Sleep Onset), sleep stage, etc.) of the user. In some examples, a recovery rate of the mental charge is modified based on the at least one sleep parameter.

In some implementations, if the user's mental stress estimation exceeds the stress threshold and sleep is detected, a second sleep-based mental charge replenishment mechanism is selected. The second sleep-based mental charge replenishment mechanism uses a different model than the first sleep-based mental charge replenishment mechanism. In some examples, a recovery rate in the second sleep-based mental charge replenishment mechanism is different from (e.g., less than) that in the first sleep-based mental charge replenishment mechanism. In some examples, an initial recovery rate in the second sleep-based mental charge replenishment mechanism is different from (e.g., less than) that in the first sleep-based mental charge replenishment mechanism for the user. In some examples, the mental energy replenishment at the current moment may be determined based on the mental energy replenishment at at least one previous moment and a recovery rate. In some examples, the recovery rate is determined based on at least one sleep parameter (e.g., a sleep Intensity, sleep duration, sleep start time, WASO (Wake After Sleep Onset), sleep stage, etc.) of the detected sleep. In some examples, a recovery rate of the mental charge is modified based on the at least one sleep parameter. In some examples, a same model is associated with the first and the second sleep-based mental charge replenishment mechanism.

In some implementations, if the user's mental stress estimation does not exceed the stress threshold and sleep is not detected, a wakefulness-based mental charge replenishment mechanism is selected. In some examples, the mental energy replenishment at moment t is based on the mental energy replenishment at at least one previous moment and at least one sleep parameter, e.g., sleep Intensity. In some examples, a model is associated with the wakefulness-based mental charge replenishment mechanism different from that associated with the sleep-based mental charge replenishment mechanisms. In some examples, a recovery rate associated with the wakefulness-based mental charge replenishment mechanism is less than that associated with the sleep-based mental charge replenishment mechanisms for the user.

In some implementations, if the user's mental stress estimation exceeds the threshold and sleep is not detected, it is determined that the user is in a mental charge consumption state.

### Determination of Physical Charge

The physical recovery of the user may be calculated based on heart rate reserve (HRR) and movement data (e.g., ACC data). In some implementations, the user's physical charge may be determined based on the user's exertion metric. In some examples, an exercise power may be used as the exertion metric. For example, the user's exercise power may be determined based on the user's movement data. The physical charge consumption during exercise may be determined by exercise intensity and overall activity level. For instance, exercise intensity and exertion metric may be used to determine physical charge consumption based on overall activity levels (higher intensities lead to faster decay).

In some implementations, the user's physical charge may be obtained using a model (e.g., a model based on the critical point algorithm) based on the user's heart rate reserve and movement data.

In some implementations, the physical charge of the user may be updated by at least one of comparing HRR of the user with a HRR threshold, comparing a movement level with a movement threshold, or determining whether a sleep event is detected.

In some implementations, based on at least one of the user's heart rate reserve, movement data, or sleep state, it may be determined whether the user's physical charge is in an energy replenishment or consumption state.

In some implementations, if at least one of the user's heart rate reserve does not exceed a HRR threshold, movement level does not exceed a movement threshold, and sleep is detected, it is determined that the user's physical charge is in a replenishment (or recovery) state.

In some implementations, the change amount or rate of the user's physical charge may be determined based on the user's HRR, which may be the original HRR or adjusted HRR.

In some implementations, the change amount or rate of user's physical charge may be determined based on the user's HRR and previous energy consumption parameter.

In some implementations, the HRR of the user may be adjusted according to exertion metric; for example, the HRR is amplified at higher exertion metric and reduced at lower exertion metric.

In some implementations, the change amount or rate of user's physical charge may be determined based on at least one sleep parameter of the user.

In some implementations, the change amount or rate of the user's physical charge may be corrected based on at least one sleep parameter of the user.

In some implementations, a user in a sleep state has a higher change rate of the physical charge than that for a user not in a sleep state.

In some implementations, multiple physical charge replenishment (or recovery) mechanisms may be determined, such as a sleep-based physical charge replenishment (or recovery) mechanism and a rest-based physical charge replenishment (or recovery) mechanism. Optionally, different physical charge replenishment (or recovery) mechanisms are associated with different change rates.

In some implementations, a target physical charge replenishment (or recovery) mechanism may be selected from multiple preset physical charge replenishment (or recovery) mechanisms based on at least one of sleep data (e.g., whether sleep is detected) or the user's previous physical charge replenishment parameter. In some examples, the replenishment rate of the user at the moment t is determined based on the HRR of the user and the replenishment rate of at least one previous moment. In some examples, different preset physical charge replenishment (or recovery) mechanisms are associated with different recovery rates. In some examples, different preset physical charge replenishment (or recovery) mechanisms are associated with different initial recovery rates. In some examples, different preset physical charge replenishment (or recovery) mechanisms are associated with a same or different models (e.g., different algorithms).

In some implementations, if HRR does not exceed the HRR threshold and sleep is detected, regardless of the movement level, it is determined that the user is in a physical charge recovery state, and a sleep-based physical charge replenishment (or recovery) mechanism is selected.

In this case, the recovery rate of the physical charge may be determined based on at least one of the user's previous physical charge replenishment, current HRR, or at least one sleep parameter (e.g., sleep duration, sleep start time, sleep stage, WASO (wakefulness assessment after sleep onset), etc.). In some examples, a recovery rate of the physical charge is determined by modifying an initial recovery rate associated with the sleep-based physical charge replenishment (or recovery) mechanism based on the at least one sleep parameter and/or HRR of the user.

Optionally, if the user's previous physical charge replenishment is within the first preset range, a recovery rate of the physical charge is determined based on a difference between the HRR of the user at the moment t and a maximum HRR of the user, where the maximum HRR of the user may be determined based on the user's attribute information (e.g., maximum heart rate, gender, age, general health information, etc.). Optionally, if the user's previous physical charge replenishment is not within the first preset range, a recovery rate of the physical charge is determined based on a difference between the current HRR of the user and a predefined HRR threshold.

In some implementations, if the HRR of the user does not exceed the HRR threshold and sleep is not detected, regardless of movement level, it is determined that the user is in a physical charge recovery state, and a rest-based physical charge replenishment (or recovery) mechanism is selected. In this case, if the user's previous physical charge consumption is within the first preset range, the recovery rate of the physical charge may be determined based on the user's previous physical charge replenishment and/or current HRR of the user. In some examples, a recovery rate of the physical charge is determined based on a difference between the HRR of the user at the moment t and a maximum HRR of the user. Optionally, if the user's previous physical charge replenishment is not within the first preset range, the recovery rate of the physical charge may be determined based on at least one of the user's previous physical charge consumption or current HRR.

In some implementations, if HRR of the user exceeds the HRR threshold and the movement level does not exceed the movement threshold, it is determined that the user is in a physical charge recovery state. In this case, if sleep is detected, the sleep-based physical charge replenishment (or recovery) mechanism is selected.

In some implementations, if HRR of the user exceeds the HRR threshold and movement level does not exceed the movement threshold, and sleep is not detected, the rest-based physical charge replenishment (or recovery) mechanism is selected.

In some implementations, if both HRR and movement level of the user exceed corresponding thresholds and sleep is detected, it is determined that the user is in a physical charge recovery state, and the sleep-based physical charge replenishment (or recovery) mechanism is selected.

In some implementations, if both HRR and movement level of the user exceed corresponding thresholds and sleep is not detected, it is determined that the user is in a physical charge consumption state.

### Chronic Daily Stress and Exertion Metric

The exertion metric may be applied to various exercises, such as running, and can be used to measure the physical load or stress caused by the user's daily activities and exercises.

In some implementations, activity stress may be determined based on the user's activity data, exercise stress may be determined based on the user's movement data, and the user's daily stress may be determined based on the activity stress and exercise stress.

In some implementations, exercise stress may be calculated based on exercise power data. Exercise stress may be determined based on the user's movement data and maximum power of the user. For example, the user's maximum power may be estimated, the user's maximum heart rate may be determined based on the user's attribute information (e.g., age), and the user's maximum power may be determined based on the maximum heart rate. For example, the exercise power may be determined based on the user's movement data. If the exercise power exceeds the user's maximum power, exercise stress may be determined based on the maximum power. If the exercise power does not exceed the user's maximum power, exercise stress may be determined based on the current exercise power of the user.

In some implementations, activity stress may be calculated based on the user's impulse data. For example, the user's impulse may be determined based on the user's attribute information and movement data. For example, the user's current impulse may be determined based on the user's age, gender, resting heart rate, and current heart rate.

In some implementations, it may be determined whether the user's power data exists. If available, the user's power data is used to determine the user's daily stress. If not, heart rate data is used to determine the user's daily stress.

In some implementations, the user's chronic stress may be obtained based on daily stress over a specific time period, such as averaging the 14-day daily stress to obtain the user's chronic stress. Optionally, the ratio of daily stress to chronic stress may be used to calculate the exertion metric, which is used to measure the user's chronic daily stress to characterize the user's habitual exercise and activity stress levels.

In some implementations, chronic daily stress may be incorporated into all energy consumption and recovery models. Chronic daily stress levels reduce physical charge replenishment efficiency and increase consumption, so the user needs to wait for chronic daily stress levels to decrease before achieving full charging.

### User Health Score

In some implementations, a plurality of health scores, such as resting heart rate (RHR) score, heart rate variability (HRV) score, apnea-hypopnea index (AHI) score, and body temperature score are integrated into the determination of at least one of mental, physical or overall energy charge as post-processing steps to enhance energy assessment. Given the discrete nature of these scores and their limitations in sleep scenarios, the post-processing module applies them to both wake and sleep states as general indicators of overall health.

In some examples, the total health score is a weighted sum of each health score, the weights for respective health scores may be preset, or determined based on the respective health scores.

In some other implementations, focus may be placed on RHR and HRV, with AHI and temperature removed from the calculation. Additionally, the calculation may be modified to expand the range of RHR and HRV scores to cover the entire 0-100 range. Specifically, conditional scaling and cubic or polynomial transformations may be applied to assign scores below 60 to RHR-HRV results below 85.

In some implementations, a sleep parameter may be used to modify the total and/or respective health score components to obtain revised health scores, which may be used for adjustment to at least one of mental, physical, or overall energy charge. Thus, revised health scores have a more direct impact by directly regulating at least one of mental, physical, or overall energy charge.

The previous day's health score may be applied to daytime data, while the current day's health score may be applied to night's data when new estimates are generated. To avoid abrupt increases or decreases in the user's physical and/or mental charge due to daily fluctuations in the health score, the health score may be used as a regulator of mental and physical charge changes through a parabolic transformation. Specifically, minute-by-minute charge changes may be regulated according to the overall health score.

In some implementations, in determining mental charge, the initial consumption may be determined based on the user's attribute information, and the overall health score may be applied through a parabolic nonlinear transformation during the determination of mental charge. In particular, minute-by-minute energy charge changes may be adjusted according to the overall health score.

In some implementations, in determining physical charge, the energy difference from the previous physical charge value is penalized using a parabolic nonlinear transformation based on the overall health score.

### Determination of Overall Energy

In some implementations, the user's overall energy may be determined based on the user's physical and mental charge. In some implementations, the user's overall energy may be determined based on any of the implementations described herein for determining the user's physical and mental charge.

In some implementations, the overall energy charge of the user is determined based on the physical and mental charge and their weights. In some examples, the overall energy charge follows an exponential aggregation mechanism similar to overall health scores.

A conclusion score for the user may be obtained based on the above energy components, such as based on physical charge and mental charge, or based on physical charge, mental charge, and overall energy, or based on overall energy charge at different moments, or based on at least one of physical charge, mental charge, overall energy charge, or overall health score. For example, the conclusion score may be obtained by weighted averaging of each energy component. In some implementations, the weight of each energy component may be determined based on the value of each energy component, or based on the user's subjective input, or based on the user's subjective assessment of each energy component, etc.

In some implementations, energy reports may be provided to the user, such as real-time energy reports, timed energy reports, event-based energy reports, energy report summaries for specific time periods (e.g., daily, weekly, and/or monthly), etc. The energy report may include real-time energy (e.g., at least one of physical charge value, mental charge value, and overall energy value), charge changes (e.g., consumption and/or replenishment values of at least one of physical charge, mental charge, and overall energy), conclusion scores, energy statistics (minimum overall energy value and/or maximum overall energy value and corresponding at least one of physical and mental charge), change rates (speed, frequency, and efficiency of energy replenishment and/or consumption, and corresponding physical and mental components), energy resilience (e.g., characterizing recovery of physical and/or mental charge after exposure to physical and/or mental stress), suggestions or guidance.

In some implementations, interactive conversations with a chatbot may be provided: leveraging the power of large language models (LLMs), a domain knowledge base and metrics have been curated, covering all components of psychological, physiological, and overall charging, as well as readiness scores and conclusion scores. Users can use natural language to ask questions about domain knowledge or insights related to their energy (equivalent to charge), readiness score, conclusion score, or their components.

This disclosure provides a method for dynamically determining the energy level of a user, including: the processor obtains one or more sensor signals or physiological signals associated with said user; The processor determines the estimation of the user's physical and mental power based on the sensor signals or physiological signals, wherein the user's body power is related to the user's physical energy consumption, physical energy recovery, or both, and the user's mental power is associated with the user's mental energy consumption, mental energy recovery, or both; and the processor determines the energy power of the user based on the estimation of the user's physical and mental power.

In some embodiments, the energy power of the user is determined in real time as an energy power value associated with the energy level of the user.

In some embodiments, the energy power of the user is determined based on the estimation of the user's physical and mental power, comprising: based on the sensor signal or physiological signals, the detected activity or activities are determined; Based on the detected activity or activities, adjust the initial energy power of the user to obtain the energy power of the user.

In some embodiments, the user's body charge is determined based on one or more of the user's heart-related indicators and one or more exercise measurements acquired by one or more sensors, and one or more heart-related indicators are associated with the user's ability to return to a normal heart rate.

In some embodiments, the energy power of the user is determined according to the estimation of the user's physical and mental power, comprising: determining at least one of the user's fatigue index data and sleep data based on one or more sensor signals or physiological signals, and the sleep data includes the sleep state; Based on at least one of the user's exertion indicator data and sleep data, determine that the user is in a state of physical energy expenditure or physical energy recovery.

In some embodiments, the energy power of the user is determined based on the estimation of the user's physical and mental power, comprising: determining the user's stress level and at least one of the sleep data based on one or more sensor signals or physiological signals, and the sleep data includes sleep state; At least partially, determine that the user is in a state of mental energy expenditure or mental energy recovery, at least in part based on at least one of the user's stress levels and sleep data.

In some embodiments, the method also comprises: determining the health assessment data of the user based on one or more sensor signals or physiological signals; and adjusting at least one of the physical and mental levels of said user based on said user's health assessment data.

In some embodiments, the health assessment data of said user is obtained based on at least one of the user's cardiac data, respiratory data, and body temperature data.

In some embodiments, the cardiac data of said user includes at least one of the user's heart rate variability, arrhythmia indication, and resting heart rate.

In some embodiments, the user's respiratory data includes at least one of the user's apnea-hypopnea index and sleep apnea indications.

In some embodiments, the estimate of the user's physical and mental power is determined based on the sensor signal or physiological signals, comprising: based on the sensor signal or physiological signals, the detection of a predefined event; and in response to the detection of the occurrence of the predefined event, at least in part, based on the detected predefined event, to determine the estimate of the user's physical and mental power.

In some embodiments, the predefined event includes at least one of the significant or acute stress events, supplemental sleep events, and exercise events.

In some embodiments, the estimate of the user's physical and mental power based on the sensor signal or physiological signals is determined, comprising: based on one or more sensor signals or physiological signals, the fatigue index is determined for the first time period, wherein the fatigue index is associated with the user's daily activities and exercise activities; and determine, at least in part, an estimate of the physical and mental power of said user based on said exertion indicator.

In some embodiments, the estimation of the user's physical and mental power is determined based on one or more sensor signals or physiological signals, comprising: based on one or more sensor signals or physiological signals, the fatigue index of the user in the first time period is determined; The user's exertion indicators are tracked in the second time period to obtain the user's chronic daily stress; and determining estimates of the physical and mental levels of said users, at least in part, based on the chronic daily stress of said users.

In some embodiments, the method also comprises: prompting the user to input a subjective estimate of at least one of the user's energy power, body electricity and mental power; and the processor adjusts the user's energy power based on the subjective evaluation of at least one of the energy power, body electricity, and mental power input by the user.

In some embodiments, the method further comprises: determining whether the user has completed the recovery activity; and in response to the user having completed the recovery activity, the processor adjusts the energy power of the user according to the completion of the recovery activity.

In some embodiments, the method also comprises: providing guidance to the user according to the user's energy power, wherein the guidance includes at least one of the following: information related to the energy power, information associated with the user's physical electricity estimation, information related to the user's mental power estimation, instructions on how to respond to potential situations related to the energy power, and recommended activities.

This disclosure provides a method for dynamically determining the energy level of a user, including: the processor obtains one or more sensor signals or physiological signals associated with said user; the processor detects predefined events associated with the user based on the sensor signal or physiological signals; and the processor updates the user's energy level based on the detected predefined events.

In some embodiments, the update of the user's energy level comprise: modifying the previously stored energy value associated with the user in real time based on one or more event parameters associated with the predefined event.

In some embodiments, the processor updates the user's energy power based on the detected predefined events, including: determining the estimation of the user's physical and mental energy based on the detected predefined events, wherein the user's body power is associated with the user's physical energy consumption, body energy recovery, or both, the user's mental power is related to the user's mental energy consumption, mental energy recovery, or both; Update the energy power of said user based on the estimate of the user's physical and mental power.

In some embodiments, the processor updates the energy power of the user according to the detected predefined event, comprising: determining the rate of change of the user's energy power according to the detected predefined event; Update the energy power of the user based on the change rate of the user's energy power.

In some embodiments, the method also comprises: obtaining the health assessment data of the user based on one or more sensor signals or physiological signals, and the health assessment data includes or is based on at least one of the heart health data, respiratory health data, and body temperature data; Wherein, the update of the user's energy charge is based on the detected predefined events and the user's health assessment data.

In some embodiments, the processor updates the energy power of the user according to the detected predefined events, comprising: obtaining the health assessment data of the user based on the detected predefined events; Update the energy level of said user based on the health assessment data of said user.

In some embodiments, the processor updates the user's energy charge based on the detected predefined event, including: determining that at least one of the user's physical and mental charges is in a state of energy consumption or energy recovery based on the detected predefined events; update at least one of the user's physical and mental batteries according to at least one of the user's physical and mental charges in a state of energy consumption or energy recovery; Update the user's energy charge according to at least one of the user's updated physical and mental power.

In some embodiments, the processor updates the energy power of the user according to the detected predefined event, comprising: responding to the detected predefined event as a sleep event, determining that at least one of the user's energy power, mental power and body power is in the state of energy replenishment; update the energy power of the user according to at least one update model corresponding to the energy replenishment state.

In some embodiments, the predefined event includes a sleep event and a low-activity or low-stress event in the waking state, and the energy recovery rate associated with the sleep event is greater than the energy recovery rate associated with the low-activity or low-stress event in the waking state; or the sleep event and the low activity or low stress event in the waking state correspond to different energy recovery models, and the energy recovery model is used to update the energy power of the user.

In some embodiments, the processor updates the user's energy charge based on the detected predefined event, including: in response to the detected predefined event as an exercise event or a daily activity event, determining at least one of the user's real-time heart rate reserve and activity level based on one or more sensor signals or physiological signals; Based on the comparison of at least one of the user's real-time heart rate reserve and activity level with at least one threshold, determine that the user's energy charge includes body power in a state of energy consumption or energy recovery.

In some embodiments, the user's real-time heart rate reserve is based on the user's real-time heart rate and current exertion indicators; or the user's real-time heart rate reserve is adjusted to the user's initial heart rate reserve based on the user's current exertion indicator, and the user's initial heart rate reserve is based on the user's real-time heart rate.

In some embodiments, the processor updates the energy power of the user according to the detected predefined events, comprising: obtaining an assessment of the user's body load based on the detected predefined events; Based on the assessment of said user's physical load, determine the user's real-time heart rate reserve; Update the energy level of said user based on the user's real-time heart rate reserve.

In some embodiments, the processor updates the energy power of the user according to the detected predefined event, comprising: responding to the detected predefined event as an acute or significant stress event, determining that the user's physical energy and mental energy are in an energy consumption state; update the energy power of the user based on the update model corresponding to the energy consumption state.

In some embodiments, the processor updates the energy power of the user according to the detected predefined event, comprising: determining the evaluation of the user's stress level based on the detected predefined event; Update the energy of said user based on the assessment of the user's stress level and the comparison of pressure thresholds.

In some embodiments, the evaluation of the user's stress level is determined based on the detected predefined events, comprising: determining the user's fatigue index data and at least one of the chronic daily stresses based on the detected predefined events, and determining the initial assessment of the user's stress level; Adjust for the initial assessment of the user's stress level based on the user's exertion indicator data and at least one of the chronic daily stress.

In some embodiments, the energy power of the user is updated according to the evaluation of the user's stress level and the comparison of the pressure threshold, comprising: in response to determining the user's sleep state as awake, determining that the user's mental power is in a state of energy recovery or energy consumption according to the evaluation of the user's stress level and the comparison of the pressure threshold; Or in response to determining that the user's sleep state is asleep, determining the recovery model or recovery rate associated with the user's mental power based on the assessment of the user's stress level and the comparison of stress thresholds.

In some embodiments, the processor updates the user's energy charge based on the detected predefined event, including: updating the user's energy level based on the detected predefined events and the user's chronic daily stress; or update the user's chronic daily stress based on the detected predefined events, and update the user's energy and electricity based on the user's updated chronic daily stress; or the rate of change in at least one of the mental and physical energies included by the energy charge of said user based on said user's chronic daily stress.

In some embodiments, the processor updates the energy power of the user according to the detected predefined event, comprising: according to the detected predefined event, the evaluation of the user's physical load is obtained; Determine the assessment of the user's stress level based on the assessment of the user's physical load; Update the energy charge of said user or the mental charge included in the energy charge based on the assessment of the stress level of said user.

In some embodiments, the processor updates the energy power of the user according to the detected predefined events, comprising: determining the target energy update model from a plurality of preset energy update models based on the detected predefined events; The target energy update model is used to update the energy power of the user.

In some embodiments, the predefined event includes at least one of the sleep events, rest events, significant or acute stress events, supplemental sleep events, daily activity events, and exercise events; or said predefined event includes at least one event that triggers at least one physical and mental exertion or recovery of said user.

In some embodiments, the consumption of the user's mental energy occurs only during significant or acute stressful events; or the rate of change in at least one of the user's energy, mental and physical energy is based on the user's current energy value.

This disclosure provides a method for dynamically determining the energy level of a user, including: the processor obtains one or more sensor signals or physiological signals associated with said user; The processor determines the estimation of the user's chronic daily stress based on the sensor signal or physiological signals; and the processor determines the user's energy charge based on the estimation of the user's chronic daily stress.

In some embodiments, the processor determines the estimation of the user's chronic daily stress based on one or more sensor signals or physiological signals, comprising: determining the assessment of the user's physical load based on one or more sensor signals or physiological signals; The assessment of the user's physical load is tracked and accumulated, and an estimate of the user's chronic daily stress is obtained.

In some embodiments, the processor determines the estimation of the user's chronic daily stress based on one or more sensor signals or physiological signals, comprising: obtaining the user's fatigue index data for each time period in a plurality of time periods based on the sensor signals or physiological signals; Based on the data of the user's fatigue indicators in each period of the plurality of time periods, an estimate of the user's chronic daily stress is obtained.

In some embodiments, the processor determines the user's energy power based on the estimation of the user's chronic daily stress, comprising: determining at least one sleep data of the user based on the estimation of the user's chronic daily stress, and at least one sleep data including sleep quality; Based on at least one sleep data of said user, determine the assessment of said user's energy recovery.

In some embodiments, the processor determines the user's energy power based on the estimation of the user's chronic daily stress, comprising: determining at least one of the user's energy recovery rate and energy consumption rate based on the estimation of the user's chronic daily stress; The energy power of the user is determined based on at least one of the energy recovery rate and energy consumption rate of the user.

This disclosure provides a computing device including: non-transient memory; and the processor, which is configured to execute instructions stored in non-transient memory to implement the methods described in either of the above.

In some embodiments, the computing device also comprises: at least one sensor, wherein at least one sensor is configured to acquire one or more sensor signals or physiological signals associated with the user.

This disclosure provides a non-transient computer-readable storage medium configured to store computer programs used to dynamically determine the energy level of a user, and said computer program includes instructions that can be executed by a processor to implement any of the methods described above.

This disclosure provides a device for dynamically determining the user's energy level, including at least one module or unit used to implement any of the methods described above.

### Illustrative Embodiments

The implementations of this disclosure include a method for dynamically determining and updating an energy charge of a user. The method includes obtaining, by a processor, one or more sensor or physiological signals associated with the user. The method includes determining, by the processor, an estimation of a physical charge and a mental charge of the user based on the one or more sensor or physiological signals. The physical charge of the user is associated with physical energy consumption of the user, physical energy recovery of the user, or both. The mental charge of the user is associated with mental energy consumption of the user, mental energy recovery of the user, or both. The method includes determining, by the processor in real-time, the energy charge of the user based on the estimation of the physical charge and the mental charge of the user.

In some implementations, the method further includes updating, by the processor in real-time, the energy charge of the user in response to detection of a predefined event associated with the user. The predefined event includes at least one of a significant or stress event, a supplemental sleep event, or a workout event. Updating the energy charge of the user includes dynamically modifying a value of the energy charge based on one or more event parameters associated with the predefined event detected.

In some implementations, updating the energy charge of the user includes modifying, in real-time, a previously stored energy value associated with the user according to at least one of the one or more event parameters associated with the predefined event.

In some implementations, the physical charge of the user is determined based on one or more heart-related metrics associated with the user and one or more movement measurements obtained from at least one sensor. The one or more heart-related metrics and the one or more movement measurements are indicative of energy expenditure, energy recovery, or both.

In some implementations, the mental charge of the user is determined based on at least one of a stress index, a circadian index, or a sleep quality index associated with the user.

In some implementations, the method further includes determining, by the processor, a change rate for at least one of the physical charge or the mental charge. Updating the energy charge of the user is based on the change rate determined.

In some implementations, the energy charge of the user is continuously recomputed during a time window to generate an instantaneous energy level curve representing a real-time energy fluctuation of the user.

In some implementations, determining the energy charge of the user based on the estimation of the physical charge and the mental charge of the user includes determining whether the user is in a state of physical energy depletion or a state of physical recovery based on at least one of an exertion metric or sleep data that is obtained based on the one or more sensor or physiological signals. The method includes determining whether the user is in a state of mental energy depletion or a state of mental energy repletion based at least in part on at least one of a stress level or the sleep data that is obtained based on the one or more sensor or physiological signals.

In some implementations, the method further includes obtaining a health estimation metric of the user based on the one or more sensor or physiological signals. The health estimation metric is obtained based on at least one of heart health, breathing health, or body temperature of the user. The method includes modifying at least one of the physical charge or the mental charge based on the health estimation metric.

In some implementations, determining the estimation of the physical charge and the mental charge of the user based on the one or more sensor or physiological signals includes determining an exertion metric of a first time period based on the one or more sensor or physiological signals. The exertion metric is associated with both daily activities and workout activities of the user. The estimation of the physical charge and the mental charge is determined based at least in part on the exertion metric.

In some implementations, determining the estimation of the physical charge and the mental charge of the user based on the one or more sensor or physiological signals includes determining an exertion metric of a first time period based on the one or more sensor or physiological signals. The method includes tracking the exertion metric over a second time period to obtain a chronic stress metric. The estimation of the physical charge and the mental charge is determined based at least in part on the chronic stress metric.

In some implementations, the method further includes prompting the user to input whether the user completed a recovery activity. Responsive to the user inputting that the recovery activity is completed, the processor updates the energy charge based on completion of the recovery activity.

In some implementations, updating the energy charge of the user includes integrating an exertion metric obtained over a first period of time and a chronic stress metric obtained over a second period of time. The second period of time is greater than the first period of time.

The implementations of this disclosure also include a method for dynamically determining and updating an energy charge of a user. The method includes obtaining, by a processor, one or more sensor or physiological signals associated with the user. The method includes determining, by the processor in real-time, an initial energy charge of the user. The initial energy charge of the user is based on an estimation of a physical charge and a mental charge of the user. The physical charge and the mental charge are estimated based on the one or more sensor or physiological signals. The method includes detecting, by the processor, a predefined event associated with the user based on the one or more sensor or physiological signals associated with the user. The method includes updating, by the processor, the energy charge of the user based on the predefined event detected.

In some implementations, the predefined event includes at least one of a significant or stress event, a supplemental sleep event, or a workout event.

The implementations of this disclosure also include a computing device for dynamically determining and updating an energy charge of a user. The computing device includes a non-transitory memory and a processor configured to execute instructions stored in the non-transitory memory. The processor is configured to obtain one or more sensor or physiological signals associated with the user. The processor is configured to determine an estimation of a physical charge and a mental charge based on the one or more sensor or physiological signals. The physical charge of the user is associated with physical energy consumption of the user, physical energy recovery of the user, or both. The mental charge of the user is associated with mental energy consumption of the user, mental energy recovery of the user, or both. The processor is configured to determine the energy charge of the user in real-time based on the estimation of the physical charge and the mental charge. The processor is configured to detect a predefined event associated with the user based on the one or more sensor or physiological signals. The processor is configured to update in real-time the energy charge of the user in response to detecting the predefined event. Updating the energy charge of the user includes dynamically modifying a value of the energy charge based on one or more event parameters associated with the predefined event detected.

In some implementations, the computing device further includes at least one sensor wearable by the user. The at least one sensor is configured to obtain the one or more sensor or physiological signals associated with the user.

In some implementations, the at least one sensor is located on or part of a wristwatch that is configured to be worn by the user.

In some implementations, updating the energy charge of the user includes integrating an exertion metric obtained over a first period of time and a chronic stress metric obtained over a second period of time. The second period of time is greater than the first period of time.

In some implementations, the processor is further configured to execute instructions stored in the non-transitory memory to determine an estimation of at least one sleep metric associated with the user based on the one or more sensor or physiological signals. Determining the energy charge of the user is further based on the estimation of the at least one sleep metric.

Technical specialists skilled in the art should understand that the implementations in this disclosure may be implemented as methods, systems, or computer program products. Therefore, this disclosure may be implemented in forms of a complete hardware implementation, a complete software implementation, and a combination of software and hardware implementation. Further, this disclosure may be embodied as a form of one or more computer program products which are embodied as computer executable program codes in computer writable storage media (including but not limited to disk storage and optical storage).

This disclosure is described in accordance with the methods, devices (systems), and flowcharts and/or block diagrams of computer program products of the implementations, which should be comprehended as each flow and/or block of the flowcharts and/or block diagrams implemented by computer program instructions, and the combinations of flows and/or blocks in the flowcharts and/or block diagrams. The computer program instructions therein may be provided to generic computers, special-purpose computers, embedded computers or other processors of programmable data processing devices to produce a machine, wherein the instructions executed by the computers or the other processors of programmable data processing devices produce an apparatus for implementing the functions designated by one or more flows in the flowcharts and/or one or more blocks in the block diagrams.

The computer program instructions may be also stored in a computer readable storage which is able to boot a computer or other programmable data processing device to a specific work mode, wherein the instructions stored in the computer readable storage produce a manufactured product containing the instruction devices which implements the functions designated by one or more flows in the flowcharts and/or one or more blocks in the block diagrams.

The computer program instructions may also be loaded to a computer or another programmable data processing device to execute a series of operating procedures in the computer or the other programmable data processing device to produce a process implemented by the computer, whereby the computer program instructions executed in the computer or the other programmable data processing device provide the operating procedures for the functions designated by one or more flows in the flowcharts and/or one or more blocks in the block diagrams.

Apparently, the technical specialists skilled in the art may perform any variation and/or modification to this disclosure by the principles and within the scope of this disclosure. Therefore, if the variations and modifications herein are within the scope of the claims and other equivalent techniques herein, this disclosure intends to include the variations and modifications thereof.

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising,” and variations thereof as used herein is used synonymously with the term “including” and variations thereof and are open, non-limiting terms. The terms “optional” or “optionally” used herein mean that the subsequently described feature, event, or circumstance may or may not occur, and that the description includes instances where said feature, event or circumstance occurs and instances where it does not. The terms “at least one of A or B,” “at least one of A and B,” “one or more of A or B,” “A and/or B” used herein mean “A,” or “B” or “A and B.”

While the disclosure has been described in connection with certain embodiments or implementations, it is to be understood that the disclosure is not to be limited to the disclosed embodiments but, on the contrary, is intended to cover various modifications and equivalent arrangements included within the scope of the appended claims, which scope is to be accorded the broadest interpretation so as to encompass all such modifications and equivalent structures as is permitted under the law.

What is claimed is:

1. A method for dynamically determining and updating an energy charge of a user, the method comprising:
obtaining, by a processor, one or more sensor or physiological signals associated with the user;
determining, by the processor, an estimation of a physical charge and a mental charge of the user based on the one or more sensor or physiological signals, wherein the physical charge of the user is associated with physical energy consumption of the user, physical energy recovery of the user, or both, and the mental charge of the user is associated with mental energy consumption of the user, mental energy recovery of the user, or both; and
determining, by the processor in real-time, the energy charge of the user based on the estimation of the physical charge and the mental charge of the user.

2. The method of claim 1, further comprising:
updating, by the processor in real-time, the energy charge of the user in response to detection of a predefined event associated with the user, wherein the predefined event includes at least one of a significant or stress event, a supplemental sleep event, or a workout event, and wherein updating the energy charge of the user includes dynamically modifying a value of the energy charge based on one or more event parameters associated with the predefined event detected.

3. The method of claim 2, wherein updating the energy charge of the user includes modifying, in real-time, a previously stored energy value associated with the user according to a least one of the one or more event parameters associated with the predefined event.

4. The method of claim 1, wherein the physical charge of the user is determined based on one or more heart-related metrics associated with the user and one or more movement measurements obtained at least one sensor, the one or more heart-related metrics and the one or more movement measurements being indicative of energy expenditure, energy recovery, or both.

5. The method of claim 1, wherein the mental charge of the user is determined based on at least one of a stress index, a circadian index, or a sleep quality index associated with the user.

6. The method of claim 1, further comprising:
determining, by the processor, a change rate for at least one of the physical charge or the mental charge,
wherein updating the energy charge of the user is based on the change rate determined.

7. The method of claim 1, wherein the energy charge of the user is continuously recomputed during a time window to generate an instantaneous energy level curve representing a real-time energy fluctuation of the user.

8. The method of claim 1, wherein determining the energy charge of the user based on the estimation of the physical charge and the mental charge of the user, comprises:
determining whether the user is in a state of physical energy depletion or a state of physical recovery based on at least one of an exertion metric or sleep data that is obtained based on the one or more sensor or physiological signals, and
determining whether the user is in a state of mental energy depletion or a state of mental energy repletion based at least in part on at least one of a stress level or the sleep data that is obtained based on the one or more sensor or physiological signals.

9. The method of claim 1, further comprising:
obtaining a health estimation metric of the user based on the one or more sensor or physiological signals, wherein the health estimation metric is obtained based on at least one of heart health, breathing health, or body temperature of the user; and
modifying at least one of the physical charge or the mental charge based on the health estimation metric.

10. The method of claim 1, wherein determining the estimation of the physical charge and the mental charge of the user based on the one or more sensor or physiological signals, comprises:
determining an exertion metric of a first time period based on the one or more sensor or physiological signals, wherein the exertion metric is associated with both daily activities and workout activities of the user; and
determining the estimation of the physical charge and the mental charge based at least in part on the exertion metric.

11. The method of claim 1, wherein determining the estimation of the physical charge and the mental charge of the user based on the one or more sensor or physiological signals, comprises:
determining an exertion metric of a first time period based on the one or more sensor or physiological signals;
tracking the exertion metric over a second time period to obtain a chronic stress metric; and
determining the estimation of the physical charge and the mental charge based at least in part on the chronic stress metric.

12. The method of claim 1, further comprising:
prompting the user to input whether the user completed a recovery activity; and
responsive to the user inputting that the recovery activity is completed, updating, by the processor, the energy charge based on completion of the recovery activity.

13. The method of claim 1, wherein updating the energy charge of the user includes integrating an exertion metric obtained over a first period of time and a chronic stress metric obtained over a second period of time, wherein the second period of time is greater than the first period of time.

14. A method for dynamically determining and updating an energy charge of a user, the method comprising:
obtaining, by a processor, one or more sensor or physiological signals associated with the user;
determining, by the processor in real-time, an initial energy charge of the user, wherein the initial energy charge of the user is based on an estimation of a physical charge and a mental charge of the user, the physical charge and the mental charge being estimated based on the one or more sensor or physiological signals;
detecting, by the processor, a predefined event associated with the user based on the one or more sensor or physiological signals associated with the user; and
updating, by the processor, the energy charge of the user based on the predefined event detected.

15. The method of claim 14, wherein the predefined event includes at least one of a significant or stress event, a supplemental sleep event, or a workout event.

16. A computing device for dynamically determining and updating an energy charge of a user, comprising:
a non-transitory memory; and
a processor configured to execute instructions stored in the non-transitory memory to:
obtain one or more sensor or physiological signals associated with the user;
determine an estimation a physical charge and a mental charge based on the one or more sensor or physiological signals, wherein the physical charge of the user is associated with physical energy consumption of the user, physical energy recovery of the user, or both, and the mental charge of the user is associated with mental energy consumption of the user, mental energy recovery of the user, or both;
determine the energy charge of the user in real-time based on the estimation of the physical charge and the mental charge;
detect a predefined event associated with the user based on the one or more sensor or physiological signals; and
update in real-time the energy charge of the user in response to detecting the predefined event, wherein updating the energy charge of the user includes dynamically modifying a value of the energy charge based on one or more event parameters associated with the predefined event detected.

17. The computing device of claim 16, further comprising: at least one sensor wearable by the user, wherein the at least one sensor is configured to obtain the one or more sensor or physiological signals associated with the user.

18. The computing device of claim 17, wherein the at least one sensor is located on or part of a wristwatch that is configured to be worn by the user.

19. The computing device of claim 17, wherein updating the energy charge of the user includes integrating an exertion metric obtained over a first period of time and a chronic stress metric obtained over a second period of time, wherein the second period of time is greater than the first period of time.

20. The computing device of claim 19, wherein the processor is further configured to execute instructions stored in the non-transitory memory to:
determine an estimation of at least one sleep metric associated with the user based on the one or more sensor of physiological signals, wherein determining the energy charge of the user is further based on the estimation of the at least one sleep metric.
